(19) United States
(12) Patent Application Publication
(10) Pub. No.: US 2026/0345384 A1
(43) Pub. Date: Nov. 5, 2026

(54) CAPSULOTOMY DEVICES, SYSTEMS, AND METHODS OF USE THEREOF

(71) Applicant: Lhotse Medical, Inc., Dallas, TX (US)

(72) Inventors: Anand DORAISWAMY, Oakland, CA (US); Kent SUZUKI, Oakland, CA (US); Azhar MEYER, Oakland, CA (US); Perry Quince ANDERSON, Kensington, CA (US)

(73) Assignee: Lhotse Medical, Inc., Dallas, TX (US)

(21) Appl. No.: 19/691,383

(22) Filed: May 28, 2026

Related U.S. Application Data
(63) Continuation-in-part of application No. PCT/US2026/017735, filed on Mar. 4, 2026.
(60) Provisional application No. 63/767,397, filed on Mar. 5, 2025.
(60) Provisional application No. 63/955,271, filed on Jan. 6, 2026.

(51) Int. Cl. A61F 9/007

(52) U.S. Cl. CPC A61F 9/007

(57) ABSTRACT

Disclosed herein is a thermal device and related methods for forming an opening in a capsular bag of an eye. The thermal device can include a heated element. The heated element can be brought into contact with or proximate the capsular bag and energized to heat the heated element to form the opening. Heat from the heated element can thermally sever tissue of the capsular bag to form the opening. The heat can locally ablate the tissue of the capsular bag to form a clean severance. The heated element can have a shape and/or size that corresponds to a target shape and/or size of the opening.

### CROSS-REFERENCE TO RELATED PATENT APPLICATIONS

This patent application is a Continuation-in-Part of International Patent Application No. PCT/US2026/017735, filed Mar. 4, 2026, which claims priority to U.S. Provisional Patent Application No. 63/767,397, filed Mar. 5, 2025, and U.S. Provisional Patent Application No. 63/955,271, filed Jan. 6, 2026. The content of each of the aforementioned patent applications is incorporated herein by reference in its entirety.

### BACKGROUND

As the eye ages, proteins in the crystalline lens begin to break down and clump together. The clumping of proteins produces a cloudy area in the crystalline lens known as a cataract that negatively impacts vision. Cataracts are a leading cause of reversible blindness worldwide. The cataract condition may be addressed with cataract surgery, wherein the crystalline lens with the cataract is removed from the eye and an intraocular lens (“IOL”) is implanted in place of the crystalline lens.

Cataract remains the leading cause of blindness worldwide. More than 90 million people are affected globally, and cataract surgery is performed over 30 million times annually. The procedure is generally safe and effective, but its quality depends critically on the integrity of the capsulotomy. A poorly executed capsulotomy can compromise intraocular lens (IOL) centration, stability, and optical performance, and may lead to complications such as posterior capsule rupture, vitreous loss, or retinal detachment.

Relevant references that may be of interest include: CA3026044; U.S. Pat. Nos. 8,814,854, 9,532,903, 11,426,308, 6,066,138, and 11,596,549; US2015/0223980; US2015/0216727; US2012/0078242; US2018/0036170; US2020/0155348; US2025/0025338; and KR20260044936.

### SUMMARY

Provided herein is a method of forming an opening in a capsular bag of an eye of a subject. The method can comprise changing a heating element into a tubular or elliptical configuration for insertion into the eye. The method can comprise heating a portion of the capsular bag with the heating element to a target temperature such that tissue of the capsular bag is severed to form the opening.

Disclosed herein is a thermal device for forming an opening in a capsular bag of an eye of a subject. The thermal device can comprise a distal portion comprising a heating element configured to be changeable into a tubular or elliptical configuration for insertion into the eye. The thermal device can comprise a power source configured to deliver energy to the heating element to heat the heating element to a target temperature such that when the heating element is positioned proximate to or in contact with tissue of the capsular bag, the tissue is severed to form the opening.

Disclosed herein is a thermal device for forming an opening in a capsular bag of an eye of a subject. The thermal device can comprise a distal portion comprising a heating element. The thermal device can comprise a power source configured to deliver pulses of energy with a voltage from about 1 V to about 20 V to the heating element to heat the heating element to a target temperature, such that when the heating element is positioned proximate to or in contact with tissue of the capsular bag, the tissue is severed to form the opening.

Provided herein is an apparatus for forming an opening in a capsular bag of an eye of a subject. The apparatus can comprise a frame. The frame can comprise a first side comprising a contact surface configured to contact the capsular bag. The frame can comprise a second side comprising a delivery interface configured to couple to a delivery device. The frame can comprise a gimbal configured to tilt the frame to maintain substantially full contact with the capsular bag within a predetermined angle range relative to an angle of the delivery device when the delivery device is coupled to the delivery interface. In some cases, the gimbal comprises one or more of: one or more posts disposed on the delivery interface and extending away from the first side; one or more hinges disposed on the delivery interface and extending away from the first side, wherein the hinges are configured to provide an open area for interfacing with the delivery device; or one or more slots proximal the one or more posts and configured to provide a slot for a distal end of the delivery device.

Provided herein is a method of forming an opening in a capsular bag of an eye of a subject. The method can comprise actuating a controller to deliver at least one current pulse with a voltage from about 1 V to about 20 V through a closed-loop conductor to a heating element. The method can comprise heating, via the heating element, a portion of the capsular bag to a target temperature such that tissue of the capsular bag is severed to form the opening.

Disclosed herein is a system for forming an opening in a capsular bag of an eye of a subject. The system can comprise a thermal device as described herein. The system can comprise a control unit configured to deliver ultrashort pulses of current to the heating element. The system can comprise a delivery device comprising a distal end and a handpiece. In some cases, the distal end of the delivery device is coupled to the heating element. In some cases, the delivery device is configured to change a shape of the heating element for insertion into the eye.

Provided herein is a robotic system for automated capsulotomy of an eye of a subject. The robotic system can comprise a robotic arm comprising an end effector. The robotic system can comprise a housing configured to be held by the end effector of the robotic arm. In some cases, the housing is coupled to a heating element configured to be changeable into a tubular or elliptical configuration for insertion into the eye. The robotic system can comprise a controller configured to deploy the heating element into an anterior chamber of the eye and to activate a current pulse to create a capsulotomy.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (also “Figure” and “FIG.” herein), of which:

FIG. 1 illustrates an example thermal device for performing a capsulotomy in accordance with example embodiments described herein.

FIG. 2 illustrates an exploded view of portions of a distal tip of a thermal device in accordance with example embodiments described herein.

FIG. 3 illustrates an assembled view of a distal tip of a thermal device, with an insulated portion shown as transparent, in accordance with example embodiments described herein.

FIG. 4 illustrates a cross-sectional view of the distal tip of a thermal device in accordance with example embodiments described herein.

FIG. 5 illustrates another view of the distal tip of a thermal device in accordance with example embodiments described herein.

FIG. 6 illustrates a cross-sectional view of another distal tip configuration for a thermal device in accordance with example embodiments described herein.

FIGS. 7A-7F illustrate perspective (FIGS. 7A-7D), top (FIG. 7E), and exploded (FIG. 7F) views of example thermal devices for performing a capsulotomy in accordance with example embodiments described herein.

FIGS. 8A-8E illustrate bottom (FIG. 8A), side (FIG. 8B), and perspective (FIGS. 8C-8E) views of portions of an example thermal device in accordance with example embodiments described herein.

FIGS. 9A-9D show perspective (FIGS. 9A, 9B, and 9D) and side (FIG. 9C) views of example gimballed contact ring configurations of an example thermal device in accordance with example embodiments described herein.

FIG. 10 illustrates a perspective view of an example thermal device in accordance with example embodiments described herein.

FIGS. 11A, 11B, and 11C illustrate perspective views of an example ring adjustment device for use with an example thermal device in accordance with example embodiments described herein.

FIGS. 11D-11V show perspective (FIGS. 11D, 11E, 11F, 11H, 11L, 11M, 11N, 11O, 11Q, 11R, 11S, 11T) and side (FIGS. 11G, 11I, 11J, 11K, 11P) views of another example ring adjustment device for use with an example thermal device in accordance with example embodiments described herein.

FIGS. 11U-11Z show perspective (FIG. 11U) and side (FIGS. 11V, 11W, 11X, 11Y, 11Z) views of another example ring adjustment device for use with an example thermal device in accordance with example embodiments described herein.

FIGS. 12A-12B illustrate perspective views of alternative ring adjustment devices for use with an example thermal device in accordance with example embodiments described herein.

FIGS. 13A-13D illustrate a method for forming an opening in a capsular bag using an example thermal device in accordance with example embodiments described herein.

FIGS. 14A-14B show contact between a contact ring of an example thermal device and a capsular bag in accordance with example embodiments described herein.

FIG. 15 illustrates an opening formed in a capsular bag during bench-top testing in accordance with example embodiments described herein.

FIG. 16A illustrates an anterior view of an opening formed in a capsular bag in accordance with example embodiments described herein.

FIG. 16B illustrates a posterior view of the opening formed in the capsular bag of the eye of FIG. 16A in accordance with example embodiments described herein.

FIGS. 17A-17B show openings formed in a capsular bag of an eye with a thermal device in accordance with example embodiments described herein.

FIG. 18 shows a graphical representation of tear strength characteristics of capsular tissue in accordance with example embodiments described herein.

FIGS. 19A-19C illustrate a perspective view (FIG. 19A) and side views (FIGS. 19B, 19C) of example ring adjustment devices for use with a thermal device in accordance with example embodiments described herein.

FIGS. 20A-20D illustrate perspective views (FIGS. 20A, 20C, 20D) and a top view (FIG. 20B) of another example ring adjustment device, including locked and unlocked configurations, in accordance with example embodiments described herein.

FIG. 21 illustrates a perspective view of another example ring adjustment device for use with a thermal device in accordance with example embodiments described herein.

FIG. 22 illustrates example ring adjustment devices for use with a thermal device in accordance with example embodiments described herein.

FIGS. 23A-23C illustrate top views (FIGS. 23A, 23B) and a perspective view (FIG. 23C) of an example thermal device in open and closed states in accordance with example embodiments described herein.

FIGS. 24A-24B illustrate an example method for forming an opening in a capsular bag using a thermal device in closed (FIG. 24A) and open (FIG. 24B) states in accordance with example embodiments described herein.

FIG. 25 illustrates a perspective view of a push-button delivery device in accordance with example embodiments described herein.

FIGS. 26A-26B illustrate top views of the push-button delivery device on a handle in open (FIG. 26A) and closed (FIG. 26B) states in accordance with example embodiments described herein.

FIGS. 27A-27D illustrate perspective views of a scroll-wheel delivery device in closed (FIGS. 27A, 27D), open (FIG. 27B), and middle (FIG. 27C) positions in accordance with example embodiments described herein.

FIGS. 28A-28E illustrate insertion (FIG. 28A), deployment (FIGS. 28B, 28C), and removal (FIGS. 28D, 28E) of a scroll-wheel delivery device during a capsulotomy procedure in accordance with example embodiments described herein.

FIGS. 29A-29F illustrate operational transitions of a scroll-wheel delivery device from open (FIGS. 29A, 29F), to middle (FIGS. 29B, 29D, 29E), and to closed (FIG. 29C) configurations in accordance with example embodiments described herein.

FIGS. 30A-30B illustrate perspective views of a scroll-wheel delivery device on a base station in accordance with example embodiments described herein.

FIGS. 31A-31C illustrate top (FIG. 31A), front (FIG. 31B), and disassembled (FIG. 31C) views of a base station in accordance with example embodiments described herein.

FIGS. 32A-32F illustrate front (FIG. 32A), side (FIGS. 32B-32C), perspective (FIGS. 32D-32E), and disassembled (FIG. 32F) views of a handle for a delivery device in accordance with example embodiments described herein.

FIGS. 33A-33E illustrate top (FIG. 33A), side (FIG. 33B), and perspective (FIGS. 33C-33E) views of a scroll-wheel delivery device with a handle in accordance with example embodiments described herein.

FIGS. 34A-34L illustrate top (FIGS. 34A, 34H), side (FIGS. 34B, 34J), perspective (FIGS. 34C-34F, 34I, 34K, 34L), and disassembled (FIG. 34G) views of a scroll-wheel delivery tip attachment in open (FIGS. 34A-34G) and closed (FIGS. 34H-34L) configurations in accordance with example embodiments described herein.

FIGS. 35A-35L illustrate top (FIGS. 35A, 35H), side (FIGS. 35B, 35C, 35I, 35J), perspective (FIGS. 35D-35F, 35K, 35L), and disassembled (FIG. 35G) views of a spring-loaded push-button delivery tip attachment in open (FIGS. 35A-35G) and closed (FIGS. 35H-35L) configurations in accordance with example embodiments described herein.

FIG. 36 illustrates an example capsulotomy specimen and eye preparation in accordance with example embodiments described herein.

FIG. 37 illustrates an example mechanical testing apparatus used to evaluate capsular tissue behavior in accordance with example embodiments described herein.

FIG. 38 illustrates a graphical representation of force-distance data associated with example capsulotomy procedures in accordance with example embodiments described herein.

FIG. 39 illustrates a graphical representation of comparative force-distance data in accordance with example embodiments described herein.

FIG. 40 illustrates a graphical representation comparing multiple measured parameters in accordance with example embodiments described herein.

FIG. 41 illustrates a graphical representation of peak force measurements in accordance with example embodiments described herein.

FIG. 42 illustrates a graphical representation of distance measurements in accordance with example embodiments described herein.

FIG. 43 illustrates a graphical representation of a thermal safety profile in accordance with example embodiments described herein.

FIG. 44 illustrates an optical micrograph of a capsulotomy edge formed using an example thermal device in accordance with example embodiments described herein.

FIGS. 45A-45B illustrate scanning electron micrographs of a capsulotomy edge formed using an example thermal device in accordance with example embodiments described herein.

FIGS. 46A-46C illustrate scanning electron micrographs of a comparator capsulotomy edge in accordance with example embodiments described herein.

FIGS. 47A-47D illustrate post-procedure images from an in vivo model in accordance with example embodiments described herein.

FIGS. 48A-48B illustrate magnified views of a capsulotomy edge following a procedure in accordance with example embodiments described herein.

FIGS. 49A-49C illustrate side views of an example heating element ring assembly without notches (FIG. 49A) and with notches (FIG. 49B and FIG. 49C).

### DETAILED DESCRIPTION

During cataract surgery, a surgeon may cut a hole, sometimes referred to as a capsulorhexis or capsulotomy, in an anterior portion of a capsular bag of the eye to access a cataracted crystalline lens. The surgeon can access and remove the crystalline lens through the hole. With the cataracted crystalline lens removed, an intraocular lens (IOL) can be inserted through the hole into the emptied capsular bag to restore a patient's sight.

A surgeon may perform anterior capsulotomy via continuous curvilinear capsulorhexis (CCC). Forming a hole in the capsular bag with a target (e.g., desired) shape and/or size can be difficult and may require substantial skill and a long learning curve. To form the hole, a surgeon may use a cutting tool, such as forceps, a needle, and/or a blade, to mechanically cut the hole. The surgeon, in some instances, can be inconsistent in cutting holes of target shape, size, or centration. The surgeon can inadvertently tear (e.g., radial tear, capsular rupture) the capsular bag in undesired ways when cutting the hole, and such tearing can compromise the structural integrity of the capsular bag. The undesired tearing can also extend to the equator or posterior capsule. In some instances, the surgeon may accidentally cut a hole that is larger or smaller than a target, which can complicate surgery. Certain cases can add additional complexities, such as, but not limited to, cases with pediatric cataracts (elastic capsule), intumescent cataracts (high intracapsular pressure), small pupils, and shallow anterior chambers. These challenges can create variability in outcomes and limit widespread access to cataract surgery, especially in regions where surgical training and infrastructure are limited.

In some cases, energy-based alternatives may exist. Energy-based alternatives can comprise Femtosecond Laser Capsulotomy, which may be highly reproducible with a programmable geometry and integration with refractive corrections. However, Femtosecond Laser Capsulotomy may use $300k-$500k capital equipment, complex optical docking, suction fixation, have a significant footprint, increase procedure time, have high maintenance costs, and depend on electrical infrastructure. As such, Femtosecond Laser Capsulotomy may be limited to high-resource settings.

Energy-based alternatives may include thermal/RF devices such as diathermy and RF-based capsulotomy devices. However, thermal/RF devices may cause significant collateral thermal damage to the eye, irregular cut edges, endothelial injury, and inconsistent performance.

Energy-based alternatives may include plasma or arc-based devices that use ionized gas or electrical arcs to cut tissue. However, plasma or arc-based devices may be limited to high-voltage operations, may have an unpredictable arc path, and may have a risk of collateral damage to the eye.

A clinical and economic gap may exist between low-cost manual techniques (unreliable, skill-dependent) and high-cost laser solutions (precise but inaccessible). Accordingly, an unmet need can exist for a capsulotomy or capsulorhexis device that can produce reliable, precise, and/or reproducible capsulotomies regardless of surgeon skill; can operate at low voltage and current for safety; can have minimal collateral thermal effect; can be compatible with microincision techniques (e.g., less than or equal to about 2 mm); can successfully work across patient populations (adult, pediatric, intumescent cataracts); and can be deployed in low-resource or ambulatory surgical centers without large footprint or suction docking.

The present disclosure relates to ophthalmic surgical devices, systems, and methods, and more particularly to energy-based devices for creating openings in the lens capsule during cataract surgery. The present disclosure introduces a novel approach to capsulotomy based on direct-current (DC) powered ultrashort pulsed thermal ablation using a low thermal mass cutting element.

The disclosed devices, systems, and methods may provide precision and reproducibility by producing capsulotomies that are substantially identical to the geometry of the loop. Unlike manual continuous curvilinear capsulorhexis (CCC), which varies with surgeon skill and intraoperative conditions, the ultrashort pulsed DC ablation method can yield a cut that is circular to within microns of deviation, reproducible across patients, surgeons, and surgical environments, independent of capsular elasticity, which may be important in pediatric and intumescent cataracts, and compatible with consistent intraocular lens (IOL) centration and stability, which may be critical for advanced IOL technologies (e.g., multifocal, toric, accommodative). In some cases, rather than DC ablation, the ablation can be conducted via alternating current (AC).

The entire capsulotomy may be completed in less than or equal to about 0.1 second. By contrast, manual CCC can take 30-60 seconds, and femtosecond lasers can use docking, imaging, and laser rastering that add minutes to the procedure. The speed of the disclosed devices, systems, and methods can reduce surgical time, anesthesia exposure, and patient movement risks.

The disclosed devices, systems, and methods may provide enhanced safety relative to the above alternatives. The disclosed devices, systems, and methods may produce minimal collateral damage, as the low thermal mass of the loop allows for no significant heat transfer beyond the immediate cutting zone. The corneal endothelium, iris, posterior capsule, and vitreous can remain unaffected. The disclosed devices, systems, and methods may avoid suction docking, which can avoid IOP spikes, subconjunctival hemorrhage, and suction-related complications of femtosecond lasers. Low-voltage operation at from about 5 Volts (V) to about 20 V DC can be inherently safer than high-voltage AC or RF systems. The loop can cool almost immediately after pulse delivery, reducing risk of delayed tissue injury.

The disclosed devices, systems, and methods may be used in various cases, including but not limited to adult cataracts (standard), pediatric cataracts (elastic capsule), intumescent cataracts (high-pressure capsule), complicated cases (small pupils, shallow chambers), and posterior capsulotomies and capsule relaxing incisions. The breadth of applications can reduce the need for multiple instruments and technologies.

Unlike alternative capsulotomy devices, the disclosed devices, systems, and methods may be handheld, battery-powered, and portable, with no capital investment beyond the unit itself. The disclosed devices, systems, and methods may further provide reduced operating costs, as the disclosed devices, systems, and methods may have no per-case licensing fees (unlike femtosecond laser disposables), no expensive optical components or cooling systems, and a reusable handpiece with optional disposable sterile loop cartridges. The reduced cost profile can make the device affordable for high-volume cataract centers and outreach programs in resource-limited settings. Because the system is compact and inexpensive, in addition to regular hospital, doctor office, or outpatient care usage, the system can be deployed in mobile eye camps, rural clinics, ambulatory surgical centers without femtosecond infrastructure, and portable environments where portability is essential. The broad deployability of the system can dramatically expand global access to cataract surgery.

The ability to create a capsulotomy in less than about 0.1 s can reduce overall surgical time. High-volume surgeons can increase throughput, lowering cost per case. In outreach or mission settings, surgical efficiency can be paramount, and the disclosed devices, systems, and methods can assist with increased efficiency.

The disclosed devices, systems, and methods may have a reduced learning curve relative to manual CCC, which may use extensive training and practice. The disclosed system allows even novice surgeons to create effective capsulotomies on the first attempt, broadening the accessibility of the surgical skill.

Heat transfer can be proportional to the thermal mass of a conductor. By reducing the conductor mass to an ultra-thin loop, a system can confine heating to the capsule-tissue interface. Direct current (DC) can avoid oscillatory heating associated with AC, allowing ultrashort pulses to be applied with predictable outcomes.

In addition to anterior capsulotomy for standard adult cataract surgeries with a circular capsulotomy of about 2 mm to about 5 mm, other applications of the devices, systems, and methods disclosed herein can include, but are not limited to, posterior capsule opening intraoperatively; reducing or eliminating the need for postoperative YAG laser; pediatric cataract surgery with reproducible elastic capsule cuts; intumescent cataracts with rapid opening to reduce radial tearing from high pressure; capsular relaxing incisions for fibrosis or capsular contraction syndrome; selective zonule release in subluxated lenses; iris procedures such as iridotomy or iridoplasty; glaucoma micro-incisions in trabecular meshwork; and corneal incisions for refractive or therapeutic purposes.

The principle of ultralow-mass DC pulsed ablation can be extended to ENT tympanic membrane fenestrations; neurosurgery dural opening; dermatology precision skin excisions; gastroenterology endoscopic resections; and veterinary ophthalmology such as animal cataract surgery.

### Robotic Use

In some cases, a key bottleneck in advancing fully robotic cataract surgery has been the capsulotomy. While phacoemulsification and lens implantation can be mechanized and standardized with relative ease, the anterior capsulorhexis may remain a highly manual and skill-dependent procedure. Robotic platforms and experimental systems have struggled to reproduce the nuanced hand motions, variable tearing forces, and real-time adjustments required to perform a continuous curvilinear capsulorhexis. The capsulotomy limitation has restricted the automation of cataract surgery, preventing widespread robotic adoption despite rapid progress in other surgical domains.

Various elements of the devices, systems, and methods disclosed herein can lend themselves to robotic integration and use in robotic cataract platforms. The devices, systems, and methods disclosed herein can comprise a smart feedback control system with thermal loop resistance monitoring to auto-terminate pulses.

The devices, systems, and methods disclosed herein can remove the need for fine manual tearing maneuvers. By substituting manual rhexis with a low-mass, direct-current, ultrashort pulsed ablation loop, capsulotomy can become a one-action, geometry-determined process. The loop itself can dictate the size and shape of the opening, and the ultrashort pulse allows the execution to be both instantaneous and reproducible across patients. Instead of replicating complex human dexterity, a robotic system can position the loop and trigger the pulse-tasks that are vastly simpler to automate with high reliability.

From a systems engineering perspective, the disclosed approach can simplify the robotic workflow. Instead of programming a robot to execute circular tearing with millimeter-scale accuracy under varying anterior chamber dynamics, the robot can simply deliver a pre-shaped loop, deploy the loop into position, and energize the loop. The disclosed approach can reduce the number of active control variables, eliminate or reduce the unpredictable tissue-tearing forces, and shrink the algorithmic complexity. In practical terms, the disclosed approach can lower the barrier to designing and scaling robotic cataract platforms that can function consistently across diverse anatomies.

Another aspect of the disclosure that lends itself to robotics can be the predictability of outcomes across patient variability. Manual capsulorhexis can be particularly challenging in pediatric eyes, intumescent lenses, and cases with weak zonules or shallow chambers. Each of these cases can use nuanced adjustments that may be hard to encode into robotic control systems. The ultrashort DC ablation method, however, can bypass these variables: the loop size defines the cut, and the cut completes so rapidly that capsular elasticity, pressure gradients, or zonular weakness become irrelevant. The predictability of the ultrashort DC ablation method can make the technique inherently scalable across the full spectrum of patient anatomies, a prerequisite for autonomous or semi-autonomous robotic deployment.

The devices, systems, and methods can also support modularity—a critical feature for future surgical robots. The devices, systems, and methods can include programmable or prefabricated loop shapes. For example, cartridges with variable geometry may be selectable intraoperatively. Loop geometries can be prefabricated in multiple sizes and shapes (e.g., 2 mm, 2.5 mm, 3 mm, 5 mm, 7 mm, or any interval therein; circular, elliptical, polygonal, square, triangular, etc.), allowing robotic systems to select and deploy the appropriate cartridge for each patient. The modular approach can match well with robotic architecture, where automated tool selection and exchange may be standard. In contrast, manual capsulorhexis can lack any inherent modularity, relying entirely on the surgeon's hand skill and judgment in real time.

By addressing the single hardest-to-automate procedure in cataract surgery, the disclosure described herein positions the ultrashort DC ablation loop as the enabler of robotic cataract surgery at scale. The ultrashort DC ablation loop can standardize the capsulotomy. The uniformity of the standardized capsulotomy can not only aid robotics but can also lay the foundation for new surgical training paradigms, where surgeons and robotic assistants alike can rely on consistent capsulotomy outcomes as a baseline.

The present disclosure can offer a compact, low-cost, low-energy, and geometry-driven method, that democratizes access to precision capsulotomy and makes the method inherently compatible with robotic execution. The present disclosure can unlock the possibility of high-volume, standardized, robot-assisted cataract surgery worldwide-an advancement toward addressing the global cataract burden.

Robotic devices and systems can be used alongside the thermal devices and heated elements described herein. In some cases, a robotic system can comprise a robotic arm. The robotic arm can comprise an end effector on a distal end of the robotic arm. The end effector can be configured and shaped to hold a thermal device as described herein. The end effector can be configured and shaped to hold a housing of a thermal device as described herein. A distal end of the housing can be coupled to a heating element (e.g., heating loop, contact frame, ring, loop, and other terminology used herein). The distal end of the housing may be coupled to the heating element via an extension arm. The extension arm can comprise the distal end of the thermal device. In some cases, the housing held by the end effector can comprise a delivery device. The delivery device can comprise a delivery device as described in the Delivery Devices section below, or an alternative delivery device. For delivery devices that require physical, manual, automatic, or handheld actuation, the robotic arm and end effector can take the place of a human hand where a physician would otherwise hold or actuate the delivery device. As disclosed herein, during delivery, the heating element can be folded into a tubular or elliptical configuration for insertion into the eye until the heating element approaches the capsular bag. The folding can be done either by compressing (e.g., squeezing) the heating element or by extending/stretching the heating element. The heating element can comprise a contact frame. The heating element can comprise a conductive loop.

A controller can be configured to deploy the heating element into the eye. The controller can also activate a current pulse toward the heating element to heat the heating element and create the thermal-based capsulotomy. In some cases, a robotic system can include 1, 2, 3, 4, 5, or more controllers. In some cases, at least one controller may be disposed in the thermal device. In some cases, at least one controller may be disposed in the robot or be otherwise configured to remotely control the robot. In some cases, at least one controller may control at least one other controller. In some cases, a thermal device controller can be configured to control, or otherwise affect the robotic controller, such as by providing temperature information to the robotic controller. In some cases, the robotic controller can be configured to control the thermal device controller. In some cases, the robotic controller can be configured to command the controller to deliver at least one energy pulse to create the capsulotomy. The energy pulse can be an ultrashort direct-current pulse. The robotic controller can be configured to position the heating element in the anterior chamber of the eye.

Use of robotic systems in conjunction with the thermal devices and delivery devices described herein can be beneficial for accuracy, timeliness, and reproducibility. A robotic capsulotomy can be reproducible without manual tearing. The robotic system can apply a feedback-controlled pulse based on heating element resistance or temperature. The robotic system can perform anterior and posterior capsulotomies in the same surgical session. The robotic system can perform a capsulotomy in less than about 10 seconds, about 5 seconds, about 2 seconds, about 1 second, about 0.5 seconds, about 0.1 seconds, about 0.05 seconds, or less than about 0.01 seconds.

In some cases, robotic systems can be used along with thermal device tip kits. Each tip kit can have a variety of different sizes of pre-formed tips for selection depending on the operation and the patient (e.g., child with small eyes versus adult with larger eyes). In some cases, the heating elements can have different geometries and/or different perimeters in addition to, or as an alternative to, different sizes. Each tip can comprise one or more of a distal end of a delivery device, a contact frame, a closed-loop conductor, or a gimballed joint. The robotic system can comprise a tool-change mechanism for detaching a first tip and attaching a second tip from the same tip kit or from a different tip kit. The robotic system, via the robotic controller, can select a tip of the tip kit based on a preset tool number (e.g., a number as indicated by an overseeing physician, a preset tool number based on the operation, and/or a preset tool number based on the following step in the operation). In some cases, instead of changing the tool, the same tool can be used with alternative parameters (e.g., voltage, current, pulse). The robotic controller may associate a use of a given tip with a certain parameter (e.g., the robot can use a thin tip for pediatric cases with a lower pulse width than with a regular tip).

In some cases, the robotic system can position the heating element, delivery device, and/or thermal device under computer vision guidance. The robotic system can position the delivery device and thermal device adjacent the eye and the heating element in the eye under computer vision guidance.

The heating elements used with the robotic system can be any of the heating elements described herein. The thermal device, housing, or handpiece used with the robotic system can be any of the thermal devices and/or delivery devices described herein.

### Thermal Devices and Systems

Disclosed herein are thermal devices, systems, and methods that can address one or more of the above-identified issues. Disclosed herein is a thermal device and related methods for forming an opening in the capsular bag of an eye. The thermal device can comprise a heated element. The heated element can be brought into contact with or proximate the capsular bag and energized to heat the heated element to form a hole. The heat from the heated element can thermally sever the tissue of the capsular bag to form the hole, which can comprise forming the hole without mechanical incising or shearing. In some cases, the heat can locally ablate the tissue of the capsular bag to form a clean severance. The heated element can be energized with electrical current, such as DC current or AC current. The electrical current can be provided by a battery (e.g., rechargeable battery). The heated element can, in some cases, have a shape and/or size that corresponds to a target shape and/or size of the hole. For example, the shape can have a closed profile (e.g., include a continuous boundary), which can comprise having a continuous boundary with a cavity (e.g., recess) that is distally open disposed within the continuous boundary. The cavity can be bounded by the boundary. The heated element can have a circular shape to form a circular hole. The heated element can have a diameter of 1-7 millimeters or any value or range of values therein to form a hole of a target size. In some cases, the heated element can have a diameter less than 1 millimeter or greater than 7 millimeters. The heated element can be foldable into a tubular or elliptical configuration for insertion into an eye. The thermal devices, systems, and methods disclosed herein can enable a surgeon to consistently form holes of a target shape and/or size in the capsular bag with reduced risk of unintentional tearing of the capsular bag and/or other complications. Additionally, the thermal devices, systems, and methods disclosed herein can enable a surgeon to more quickly form holes in the capsular bag.

In some cases, the techniques described herein relate to a method of forming an anterior opening in a capsular bag, the method including: positioning a heated element of a thermal device against an anterior portion of the capsular bag; and applying direct current to the heated element to heat the heated element to a target temperature such that tissue of the capsular bag in contact with the heated element is severed to form the anterior opening in the capsular bag.

In some cases, the thermal devices disclosed herein can be powered with a DC power source. In some cases, the devices can be powered with AC power. Powering the thermal device with a DC power source, such as a battery (e.g., rechargeable), can enable the thermal device to be portable, which can provide numerous advantages. For example, the thermal device can be easily transported. The thermal device can be used in a remote setting. The thermal device can be inexpensive, which can be at least partially due to avoiding large-scale and/or high-cost fixed capital equipment.

In some cases, the thermal devices and related methods described herein can be used to form a hole in a capsular bag without exerting mechanical tension on the capsular bag. In some cases, the thermal devices and related methods described herein can be used to form a hole in the capsular bag with thermal ablation. In some cases, the thermal devices and related methods described herein can be used to form a hole in a capsular bag without mechanical incising, cutting, and/or shearing. In some cases, the thermal devices and related methods described herein can be used to form a hole in a capsular bag without mechanical stress being applied to the capsular bag when forming the hole. In some cases, thermal conduction from the heated element alone can be responsible for separating the capsule tissue along an intended cut line. In some cases, the thermal devices and related methods described herein can avoid tensile or shear force when forming a hole in the capsular bag. In some cases, the thermal devices and related methods described herein can exclude any sharp edges, blade, knife, and/or other mechanical element that mechanically incises tissue. In some cases, contact between the heated element and the capsular bag can be light contact without significant mechanical pressure applied by the heated element. In some cases, the thermal devices and related methods described herein can exclude mechanical traction. In some cases, the severance of capsular tissue can result from thermal energy alone.

In some cases, the heated element can be cooled by discontinuing the application of DC current to the heated element and/or passing an irrigation fluid across the heated element to dissipate residual heat.

In some cases, the capsular bag is not stretched and/or deformed when forming the hole therein, which can reduce (e.g., prevent) the introduction of mechanical stresses that can result in an irregular tear of the capsular bag. In some cases, the hole can be cut in the capsular bag without cutting or substantially cutting into an interior of the capsular bag with the thermal ablation being applied on the surface level of capsular tissue.

In some cases, the thermal device can comprise an integrated feedback loop that can monitor electrical resistance at the heated element to estimate capsule contact quality and modulate DC current to avoid unintentional mechanical and/or thermal trauma.

In some cases, a viscoelastic material can be introduced into the anterior chamber of the eye prior to applying thermal energy to the capsular bag, which can help to protect surrounding tissue from excess heat transfer and/or maintain stable ocular pressure during the capsulotomy. In some cases, a thermally protective polymeric film can be applied to the outer surface of the capsular bag prior to applying heat, which can reduce heat conduction beyond a targeted tissue boundary.

In some cases, the heated element can be heated and the surgeon can form a hole by moving the heated element along (e.g., through) a surface of the capsular bag to form a hole with a desired shape and size. The heated element can be a heated point. The capsulotomy path can be defined by the surgeon moving the heated element in a circular, oval, elliptical, and/or other pattern, which can customize the shape and/or size of the hole to accommodate a geometry of a selected IOL without a dedicated heated element of a corresponding size and shape. In some cases, controlling a rate of motion (e.g., such as maintaining a consistent rate of motion) of the heated element when forming the hole can regulate total heat energy imparted to the capsular tissue to achieve a smooth and/or uniform edge. In some cases, a desired capsulotomy outline can be pre-marked and/or mapped on the capsular bag using a biocompatible dye so that the surgeon can follow the outline with the heated element to form the hole. Biocompatible dyes can comprise one or more of fluorescein, indocyanine green, Rose Bengal, Lissamine green, Brilliant blue G, methylene blue, or Trypan blue.

The handle can comprise one or more controller(s), processor(s), memory with instructions, wireless communication interface(s), wired communication interface(s), user interface(s), loudspeaker(s), light source(s), power interface(s), data interface(s), and/or other features to carry out the functions described herein.

In some cases, the devices disclosed herein may comprise integrated illumination, such as but not limited to LEDs or fiber optics on the handpiece.

In some cases, the devices disclosed herein may comprise disposable all-in-one units. The units may come as preloaded sterile devices with an integrated power supply.

In some cases, thermal devices can be used along with thermal device tip kits. Each tip kit can have a variety of different sizes of pre-formed tips for selection depending on the operation and the patient (e.g., child with small eyes versus adult with larger eyes). In some cases, the heating elements can have different geometries and/or different perimeters in addition to, or as an alternative to, different sizes. Each tip can comprise one or more of a distal end of a delivery device, a contact frame of a heating element, a closed-loop conductor, or a gimballed joint.

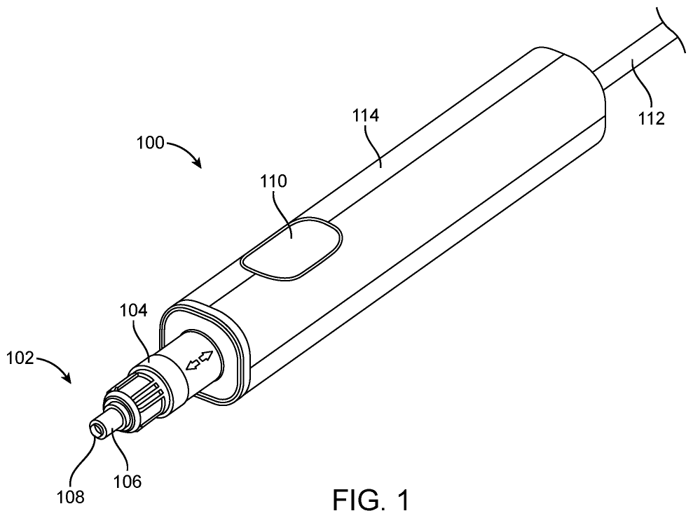

FIG. 1 illustrates a thermal device 100, which can be referred to as a thermal handpiece, thermal capsulotomy device, and/or capsulotomy device. The thermal device 100 can be used to form a hole (e.g., capsulotomy, capsulorhexis, opening, aperture) in a capsular bag of an eye of a patient (e.g., human or non-human patient).

The thermal device 100 can comprise a distal tip 102. The distal tip 102 can, in some cases, be coupled to a handle 114 (e.g., housing) of the thermal device 100. The distal tip 102 can be disposable. The distal tip 102 can be single use. The distal tip 102 can comprise an interface 104 (e.g., shank, connector, quick connect feature) that can couple with the handle 114. The interface 104 can enable a user, such as a surgeon, to quickly attach and detach distal tips 102 from the handle 114, which can be reusable (e.g., repeated use). The distal tip 102 and handle 114 can be coupled with a threaded connection, plug and socket connection, push fit connection, locking spring connection, and/or others.

A plurality of distal tips can be paired with a delivery device. The distal tips can comprise different sizes, different shapes, and/or different perimeters to fit the size of the eye and the operation. The tips can be provided individually or as a kit.

The distal tip 102 can comprise a heated element 108, which can be referred to as an element, heat-delivery tip, and/or tip. The handle 114 can comprise features to provide energy (e.g., electrical energy, direct current energy) to the heated element 108 to energize (e.g., heat) the heated element 108. For example, the handle 114 can comprise a battery (e.g., single use or rechargeable) that can provide electrical energy (e.g., direct current) that the battery applies to the heated element 108, which can heat (e.g., raise a temperature of) the heated element 108. In some cases, the electrical energy applied to the heated element 108 can resistively heat the heated element 108.

The heated element 108 can be heated to various temperatures, which can comprise 60-150 °C or any temperature or range of temperatures therein. In some cases, the heated element 108 can be heated to less than 60 °C or greater than 150 °C. The heated element 108 can be heated from about 60 degrees C to about 150 degrees C. The heated element 108 can be heated from about 60 degrees C to about 70 degrees C, about 60 degrees C to about 80 degrees C, about 60 degrees C to about 90 degrees C, about 60 degrees C to about 100 degrees C, about 60 degrees C to about 110 degrees C, about 60 degrees C to about 120 degrees C, about 60 degrees C to about 130 degrees C, about 60 degrees C to about 140 degrees C, about 60 degrees C to about 150 degrees C, about 70 degrees C to about 80 degrees C, about 70 degrees C to about 90 degrees C, about 70 degrees C to about 100 degrees C, about 70 degrees C to about 110 degrees C, about 70 degrees C to about 120 degrees C, about 70 degrees C to about 130 degrees C, about 70 degrees C to about 140 degrees C, about 70 degrees C to about 150 degrees C, about 80 degrees C to about 90 degrees C, about 80 degrees C to about 100 degrees C, about 80 degrees C to about 110 degrees C, about 80 degrees C to about 120 degrees C, about 80 degrees C to about 130 degrees C, about 80 degrees C to about 140 degrees C, about 80 degrees C to about 150 degrees C, about 90 degrees C to about 100 degrees C, about 90 degrees C to about 110 degrees C, about 90 degrees C to about 120 degrees C, about 90 degrees C to about 130 degrees C, about 90 degrees C to about 140 degrees C, about 90 degrees C to about 150 degrees C, about 100 degrees C to about 110 degrees C, about 100 degrees C to about 120 degrees C, about 100 degrees C to about 130 degrees C, about 100 degrees C to about 140 degrees C, about 100 degrees C to about 150 degrees C, about 110 degrees C to about 120 degrees C, about 110 degrees C to about 130 degrees C, about 110 degrees C to about 140 degrees C, about 110 degrees C to about 150 degrees C, about 120 degrees C to about 130 degrees C, about 120 degrees C to about 140 degrees C, about 120 degrees C to about 150 degrees C, about 130 degrees C to about 140 degrees C, about 130 degrees C to about 150 degrees C, or about 140 degrees C to about 150 degrees C. The heated element 108 can be heated to about 60 degrees C, about 70 degrees C, about 80 degrees C, about 90 degrees C, about 100 degrees C, about 110 degrees C, about 120 degrees C, about 130 degrees C, about 140 degrees C, or about 150 degrees C. The heated element 108 can be heated to at least about 60 degrees C, about 70 degrees C, about 80 degrees C, about 90 degrees C, about 100 degrees C, about 110 degrees C, about 120 degrees C, about 130 degrees C, or about 140 degrees C. The heated element 108 can be heated to at most about 70 degrees C, about 80 degrees C, about 90 degrees C, about 100 degrees C, about 110 degrees C, about 120 degrees C, about 130 degrees C, about 140 degrees C, or about 150 degrees C.

The heated element 108 can be heated from about 120 degrees C to about 150 degrees C. The heated element 108 can be heated from about 120 degrees C to about 125 degrees C, about 120 degrees C to about 130 degrees C, about 120 degrees C to about 135 degrees C, about 120 degrees C to about 140 degrees C, about 120 degrees C to about 145 degrees C, about 120 degrees C to about 150 degrees C, about 125 degrees C to about 130 degrees C, about 125 degrees C to about 135 degrees C, about 125 degrees C to about 140 degrees C, about 125 degrees C to about 145 degrees C, about 125 degrees C to about 150 degrees C, about 130 degrees C to about 135 degrees C, about 130 degrees C to about 140 degrees C, about 130 degrees C to about 145 degrees C, about 130 degrees C to about 150 degrees C, about 135 degrees C to about 140 degrees C, about 135 degrees C to about 145 degrees C, about 135 degrees C to about 150 degrees C, about 140 degrees C to about 145 degrees C, about 140 degrees C to about 150 degrees C, or about 145 degrees C to about 150 degrees C. The heated element 108 can be heated to about 120 degrees C, about 125 degrees C, about 130 degrees C, about 135 degrees C, about 140 degrees C, about 145 degrees C, or about 150 degrees C. The heated element 108 can be heated to at least about 120 degrees C, about 125 degrees C, about 130 degrees C, about 135 degrees C, about 140 degrees C, or about 145 degrees C. The heated element 108 can be heated to at most about 125 degrees C, about 130 degrees C, about 135 degrees C, about 140 degrees C, about 145 degrees C, or about 150 degrees C.

The heat from the heated element 108 can be thermally conducted to tissue of the capsular bag (e.g., tissue in contact with and/or proximate the heated element 108) to thermally sever the tissue of the capsular bag to form a hole. The heated element 108 can provide localized heat to targeted tissue. The electrical energy provided by (e.g., through) the handle 114 can be pulsed to quickly heat and stop heating the heated element 108. In some cases, the handle 114 can receive electrical energy from an external source via a wired connection 112 (e.g., cord), and the handle 114 can provide the received energy to the heated element 108. The distal tip 102 can comprise electrical contacts (e.g., wires) to facilitate the conduction of electrical energy from the handle 114 to the distal tip 102 (e.g., heated element 108).

The heated element 108 can be various sizes and/or shapes to form a hole in the capsular bag of a same size and/or shape. The various sizes and shapes can enable a surgeon to bring the heated element 108 into contact with or proximate the capsular bag and heat the heated element 108 to form a hole in the capsular bag that corresponds to the size and/or shape of the heated element 108 without moving the heated element 108 along a surface of the capsular bag. The heated element 108 can have a shape with a closed profile (e.g., include a continuous boundary), which can comprise having a continuous boundary with a cavity (e.g., recess) that is distally-open disposed within the continuous boundary. The cavity can be bounded by the boundary. For example, the heated element 108 can have a circular shape to form a hole in the capsular bag with a circular shape. The heated element 108 can have a diameter of 1-7 millimeters or any value or range of values therein. For example, in some cases, the heated element 108 can have a diameter of 6, 5.5, 5, 4.5, 4, 3.5, 3, 2.5, 2, 1.5, or 1 millimeters or any value between any of the foregoing values. In some cases, the heated element 108 can have a diameter less than 1 millimeter or greater than 7 millimeters. The heated element 108 can have other shapes, such as oval or elliptical, to form holes of other shapes, such as oval or elliptical, in the capsular bag. In some cases, the heated element 108 can have an annular shape, tube shape, ring shape, and/or others. In some cases, other shapes such as rectangular shapes and triangular shapes or polygonal shapes may also be possible. The heated element 108 can comprise a material that is electrically conductive such as a metal (e.g., stainless steel, nickel titanium, etc.). In some cases, the heated element 108 can be heated and moved along a surface of the capsular bag by the surgeon to cut a hole.

The handle 114 can comprise a user interface 110, such as a button, switch, dial, slide, and/or other interface. A surgeon can interact with (e.g., press) the user interface 110 to cause the heated element 108 to be heated (e.g., direct DC current to the heated element 108). In some cases, electrical energy can only be applied to the heated element 108 when the surgeon is interacting (e.g., pressing) the user interface 110 such that, upon the surgeon ceasing to interact with (e.g., press) the user interface 110, electrical energy will not be applied to the heated element 108.

The distal tip 102 can comprise an insulated portion 106, which can be referred to as insulation, insulated shank, and/or a holder. The insulated portion 106 can hold the heated element 108. In some cases, the heated element 108 can be bonded (e.g., bonded via thermal epoxy) to the insulated portion 106. The insulated portion 106 can couple the heated element 108 to the remainder of the distal tip 102, such as the interface 104. In some cases, a proximal portion of the heated element 108 can be disposed in the insulated portion 106 and a distal portion of the heated element 108 can protrude distally outside the insulated portion 106. In some cases, the heated element 108 can be selectively retracted into the insulated portion 106. In some cases, the heated element 108 can be selectively deployed from within the insulated portion 106. In some cases, the heated element 108 can be biased to a retracted position such that the default position of the heated element 108 is retracted into the insulated portion 106. The insulated portion 106 can protect ocular features from thermal energy. The insulated portion 106 can comprise an insulating material, such as a polymer, plastic, silicone, polycarbonate (PC), polytetrafluoroethylene (PTFE), polyether-ether-ketone (PEEK), polyetherimide, polyamide-imide (PAI), polyphenylene sulfide (PPS), and/or others. The insulated portion 106 can comprise a recess and/or cavity to receive the heated element 108. In some cases, the insulated portion 106 can be 3D printed.

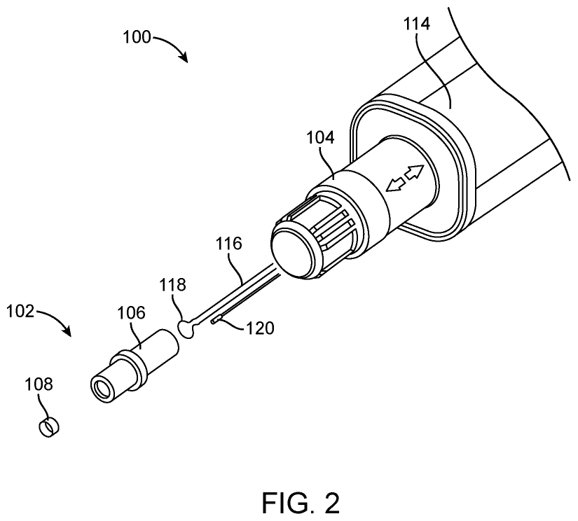

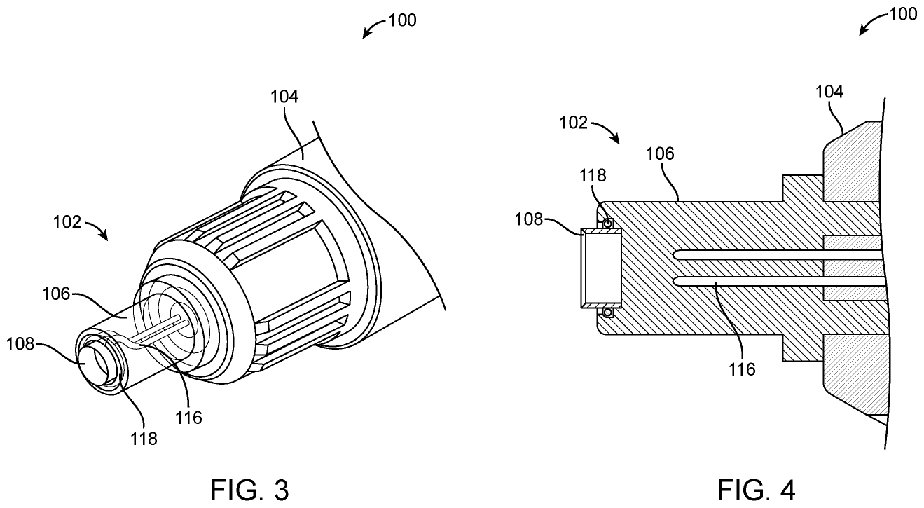

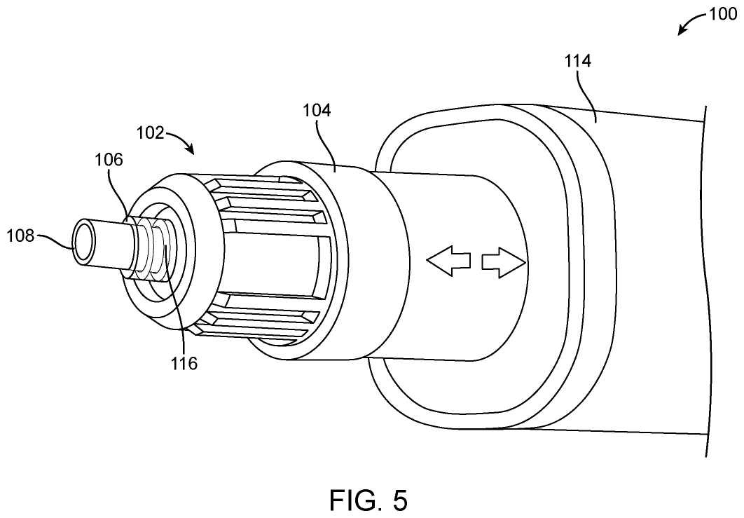

As illustrated in FIG. 2, the distal tip 102 can comprise one or more electrical conductors 116 (e.g., wires). The one or more electrical conductors 116 can deliver electrical energy to the heated element 108 to heat the heated element 108. The one or more electrical conductors 116 can direct electrical energy away from the heated element 108 after heating. The one or more electrical conductors 116 can be in contact with the heated element 108. The one or more electrical conductors 116 can comprise a contact portion 118 (e.g., loop, coil) that contacts (e.g., is disposed around, wraps around, is coupled to, is soldered to) the heated element 108 to heat the heated element 108. As illustrated in FIGS. 3-4, the one or more electrical conductors 116 can be disposed around the heated element 108. The one or more electrical conductors 116 can be disposed through lumens of the insulated portion 106. The one or more electrical conductors 116 can extend distally to the heated element 108 and extend proximally away from the heated element 108. The one or more electrical conductors 116 can be soldered to the heated element 108. The one or more electrical conductors 116 can be coupled (e.g., soldered) to electrical connectors of the handle 114. The one or more electrical conductors 116 can comprise nichrome. The one or more electrical conductors 116 can be wires. As illustrated in FIG. 5, the insulated portion 106 can, in some cases, be formed over a proximal portion of the heated element 108 and/or one or more electrical conductors 116.

As shown in FIG. 2, the distal tip 102 can comprise a temperature sensor 120 (e.g., thermocouple, thermistor). The temperature sensor 120 can be disposed proximate the heated element 108. The temperature sensor 120 can be disposed on the distal tip 102 at a location to be at or proximate the capsular bag when the thermal device 100 is in use. The temperature sensor 120 can detect temperatures. The temperature sensor 120 can facilitate closed-loop control for heating the heated element 108. For example, when the temperature sensor 120 detects an upper temperature threshold, the thermal device 100 can cease heating the heated element 108. The thermal device 100 can adjust electrical current (e.g., increase, decrease, start, stop) provided to the heated element 108 in response to detected temperatures to maintain a target temperature range during operation. The temperature sensor 120 can be coupled (e.g., soldered) to electrical connectors of the handle 114.

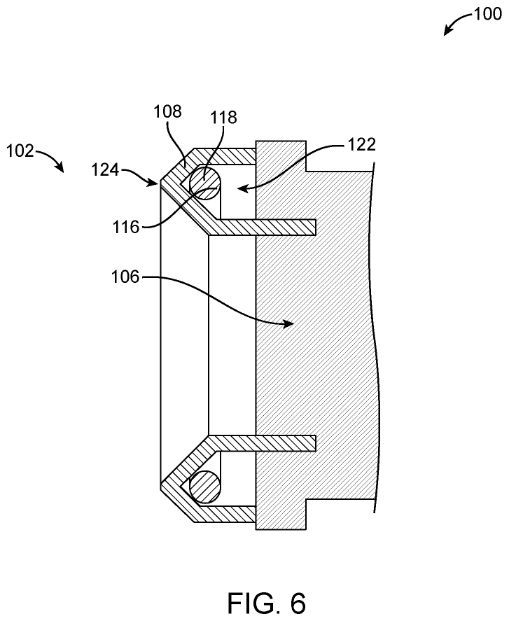

As shown in FIG. 6, in some cases, the heated element 108 can comprise an interior 122 (e.g., cavity, recess, hollow, interior cavity). The one or more electrical conductors 116 (e.g., the contact portion 118 of the one or more electrical conductors 116) can extend through the interior 122, which can comprise contacting an interior wall of the interior 122 to provide electrical energy to the heated element 108 for heating. The heated element 108, in some cases, can comprise a distal edge 124, which can be the portion of the heated element 108 to contact the capsular bag or be proximate to the capsular bag. The distal edge 124, in some cases, can be configured to be sharp to facilitate forming the hole in the capsular bag. In some cases, the distal edge 124 is not sharp. For example, the distal edge 124 can be rounded and/or blunt, which can avoid mechanical incising on the capsular bag during a procedure such that tissue separation is substantially from thermal energy and not mechanical incising.

In use, a surgeon can select a distal tip 102 with a heated element 108 that is sized and/or shaped to form a hole with a corresponding size and/or shape. For example, if the surgeon desires to form a circular hole with a 6-millimeter diameter, the surgeon can select a distal tip 102 with a heated element 108 that has a circular shape with a 6-millimeter diameter. The surgeon can attach the distal tip 102 to the handle 114. The surgeon can bring the heated element 108 into contact with or proximate an anterior portion of the capsular bag. The surgeon can interact with (e.g., push) the user interface 110, which can cause the heated element 108 to be heated to a temperature of 70-150 °C or others. As described herein, direct electrical current can be delivered from the handle 114 (e.g., power source, battery, and/or wired connection) to the heated element 108 to raise a temperature of the heated element 108, which can be via resistive heating. In some cases, a pulse (e.g., pulse modulation) of direct electrical current can be delivered to the heated element 108 which can rapidly heat the heated element 108. The heat from the heated element 108 can be conducted to the tissue of the capsular bag that is in contact with or proximate the heated element 108, which can sever the tissue of the capsular bag to form the hole. In some cases, the surgeon may not move the heated element 108 across the surface of the capsular bag because the heated element 108 has a shape and size (e.g., circular shape with a target diameter) that, when heated, can immediately form a hole of a corresponding shape and size in the capsular bag. In some cases, the surgeon may move the heated element 108 across the surface of the capsular bag to form the hole. The severed portion of the capsular bag can be removed (e.g., removed with forceps). With pulses of direct electrical current (e.g., pulse modulation), the heating and cooling of the heated element 108 can be rapid and/or controlled without dissipation to nearby regions of the capsular bag and/or other ocular features. With the hole formed, the natural lens within the capsular bag can be accessed. The natural lens can be removed and an IOL can be implanted in the emptied capsular bag through the formed hole.

### Thermal Devices and Systems Utilizing Ultrashort Pulses

The present disclosure is based on the unexpected discovery that an ultra-low thermal mass heating element, when energized with ultrashort pulses of low-voltage direct current, can produce a capsulotomy that is near instantaneous (e.g., less than or equal to about 0.1 s), precise, and safe, with negligible collateral heat spread. The low or ultra-low voltages can be from about 1 V to about 20 V. The low or ultra-low voltages can be less than 1 V. The low or ultra-low voltages can be greater than 20 V. The devices disclosed herein having such characteristics can include the devices disclosed above and below.

Some RF or diathermy devices may fail because the electrodes of those devices carry large thermal mass, storing heat and dissipating the stored heat into adjacent tissues. By contrast, when the mass of the conductive loop is minimized as in the devices disclosed herein (e.g., a nitinol wire or reduced-thickness tube having a thickness from about 25 μm to about 200 μm), the energy delivered can be localized only to the immediate cutting zone. The element can heat and cool almost instantaneously, thereby avoiding thermal conduction beyond the capsule.

In some cases, direct current (DC) can be used to power the devices disclosed herein. The power source can be a compact, low-voltage DC supply. Unlike RF or AC systems, DC can be delivered safely at less than or equal to about 20 V. DC can also allow for precise control. Ultrashort DC pulses can be tailored in duration and amplitude. DC systems can use less infrastructure than alternate systems, as DC systems may be used without RF generators, optical alignment, or cooling systems. The electrothermal ablation mechanism using localized rapid heating can lead to capsular rupture and clean circular opening while sparing nearby structures.

The heating element can comprise a loop, ring, ellipse, tube, or other circular element. In some cases, the heating element can comprise a polygonal element. The heating element can be foldable into a tubular or elliptical configuration for insertion into an eye of a subject. The heating element can be thin. A consistent loop diameter can produce a reproducible capsulotomy.

In some cases, such as when it forms a loop, the heating element can be pre-formed into a circle having a diameter from about 1 mm to about 7 mm. The loop can have a diameter from about 1 mm to about 2 mm, about 1 mm to about 2.5 mm, about 1 mm to about 3 mm, about 1 mm to about 3.5 mm, about 1 mm to about 4 mm, about 1 mm to about 4.5 mm, about 1 mm to about 5 mm, about 1 mm to about 5.5 mm, about 1 mm to about 6 mm, about 1 mm to about 6.5 mm, about 1 mm to about 7 mm, about 2 mm to about 2.5 mm, about 2 mm to about 3 mm, about 2 mm to about 3.5 mm, about 2 mm to about 4 mm, about 2 mm to about 4.5 mm, about 2 mm to about 5 mm, about 2 mm to about 5.5 mm, about 2 mm to about 6 mm, about 2 mm to about 6.5 mm, about 2 mm to about 7 mm, about 2.5 mm to about 3 mm, about 2.5 mm to about 3.5 mm, about 2.5 mm to about 4 mm, about 2.5 mm to about 4.5 mm, about 2.5 mm to about 5 mm, about 2.5 mm to about 5.5 mm, about 2.5 mm to about 6 mm, about 2.5 mm to about 6.5 mm, about 2.5 mm to about 7 mm, about 3 mm to about 3.5 mm, about 3 mm to about 4 mm, about 3 mm to about 4.5 mm, about 3 mm to about 5 mm, about 3 mm to about 5.5 mm, about 3 mm to about 6 mm, about 3 mm to about 6.5 mm, about 3 mm to about 7 mm, about 3.5 mm to about 4 mm, about 3.5 mm to about 4.5 mm, about 3.5 mm to about 5 mm, about 3.5 mm to about 5.5 mm, about 3.5 mm to about 6 mm, about 3.5 mm to about 6.5 mm, about 3.5 mm to about 7 mm, about 4 mm to about 4.5 mm, about 4 mm to about 5 mm, about 4 mm to about 5.5 mm, about 4 mm to about 6 mm, about 4 mm to about 6.5 mm, about 4 mm to about 7 mm, about 4.5 mm to about 5 mm, about 4.5 mm to about 5.5 mm, about 4.5 mm to about 6 mm, about 4.5 mm to about 6.5 mm, about 4.5 mm to about 7 mm, about 5 mm to about 5.5 mm, about 5 mm to about 6 mm, about 5 mm to about 6.5 mm, about 5 mm to about 7 mm, about 5.5 mm to about 6 mm, about 5.5 mm to about 6.5 mm, about 5.5 mm to about 7 mm, about 6 mm to about 6.5 mm, about 6 mm to about 7 mm, or about 6.5 mm to about 7 mm. The loop can have a diameter about 1 mm, about 2 mm, about 2.5 mm, about 3 mm, about 3.5 mm, about 4 mm, about 4.5 mm, about 5 mm, about 5.5 mm, about 6 mm, about 6.5 mm, or about 7 mm. The loop can have a diameter at least about 1 mm, about 2 mm, about 2.5 mm, about 3 mm, about 3.5 mm, about 4 mm, about 4.5 mm, about 5 mm, about 5.5 mm, about 6 mm, or about 6.5 mm. The loop can have a diameter at most about 2 mm, about 2.5 mm, about 3 mm, about 3.5 mm, about 4 mm, about 4.5 mm, about 5 mm, about 5.5 mm, about 6 mm, about 6.5 mm, or about 7 mm.

In some cases, loops can be folded into an ellipse or a tube for insertion through incisions less than or equal to about 1 mm. In some cases, larger loops, such as those at least about 2 mm, can be folded into an ellipse or a tube for insertion through incisions less than or equal to about 2 mm. In some cases, loops can be folded into an ellipse or a tube for insertion through incisions less than or equal to about 3 mm. In some cases, loops can be folded into an ellipse or a tube for insertion through incisions less than or equal to about 4 mm. Once inside the eye, the loop can self-deploy to its shape-memory geometry (e.g., circular or elliptical).

The devices, systems, and methods described herein can be fast and create a full, or approximately full, 360° cut in less than or equal to about 0.1 s. The devices, systems, and methods described herein can create reproducible results, with capsulotomies substantially identical to the loop shape. The devices, systems, and methods described herein are safe due to lack of suction docking, collateral heat, and endothelial damage. The devices disclosed herein can be portable. The devices can be handheld, battery-operated, and low-cost. The devices can be flexible and accommodate many geometries (e.g., circle, oval, polygon, petal, etc.) by designing the loop shape. The devices disclosed herein can be scalable and suitable for high-volume cataract centers and outreach programs.

The devices disclosed herein may comprise a DC power source, a control unit, a cutting element, a delivery handpiece, and safety and feedback components.

### Power Source Variants

The devices disclosed herein may be powered in various ways. In some cases, the devices can comprise a battery pack. The battery pack can comprise rechargeable lithium-ion, lithium-polymer, nickel-metal hydride, or alkaline batteries. In some cases, the devices can comprise an external power supply, such as but not limited to a plug-in wall adapter providing from about 1 V to about 20 V regulated DC power. In some cases, the devices can comprise an integrated capacitor bank for ultrashort, high-precision pulse delivery without continuous draw. In some cases, the devices can comprise wireless power transmission via inductive or resonant coupling, allowing tether-free handpiece use. In some cases, the devices can comprise hybrid systems comprising a rechargeable base unit with capacitor discharge into the loop. In some cases, voltage and current ranges may be adjustable. In some embodiments, the system can operate from about 1 V to about 20 V and less than about 2 Ampere (Amp) peak current, allowing for both efficacy and safety. In some cases, the system can operate at a higher current, such as but not limited to from about 15 Amp to about 20 Amp.

The devices and systems can operate at a current of from about 1 Amp to about 10 Amp. The devices and systems can operate at a current of from about 1 Amp to about 2 Amp, about 1 Amp to about 3 Amp, about 1 Amp to about 4 Amp, about 1 Amp to about 5 Amp, about 1 Amp to about 6 Amp, about 1 Amp to about 7 Amp, about 1 Amp to about 8 Amp, about 1 Amp to about 9 Amp, about 1 Amp to about 10 Amp, about 2 Amp to about 3 Amp, about 2 Amp to about 4 Amp, about 2 Amp to about 5 Amp, about 2 Amp to about 6 Amp, about 2 Amp to about 7 Amp, about 2 Amp to about 8 Amp, about 2 Amp to about 9 Amp, about 2 Amp to about 10 Amp, about 3 Amp to about 4 Amp, about 3 Amp to about 5 Amp, about 3 Amp to about 6 Amp, about 3 Amp to about 7 Amp, about 3 Amp to about 8 Amp, about 3 Amp to about 9 Amp, about 3 Amp to about 10 Amp, about 4 Amp to about 5 Amp, about 4 Amp to about 6 Amp, about 4 Amp to about 7 Amp, about 4 Amp to about 8 Amp, about 4 Amp to about 9 Amp, about 4 Amp to about 10 Amp, about 5 Amp to about 6 Amp, about 5 Amp to about 7 Amp, about 5 Amp to about 8 Amp, about 5 Amp to about 9 Amp, about 5 Amp to about 10 Amp, about 6 Amp to about 7 Amp, about 6 Amp to about 8 Amp, about 6 Amp to about 9 Amp, about 6 Amp to about 10 Amp, about 7 Amp to about 8 Amp, about 7 Amp to about 9 Amp, about 7 Amp to about 10 Amp, about 8 Amp to about 9 Amp, about 8 Amp to about 10 Amp, or about 9 Amp to about 10 Amp. The devices and systems can operate at a current of about 1 Amp, about 2 Amp, about 3 Amp, about 4 Amp, about 5 Amp, about 6 Amp, about 7 Amp, about 8 Amp, about 9 Amp, or about 10 Amp. The devices and systems can operate at a current of at least about 1 Amp, about 2 Amp, about 3 Amp, about 4 Amp, about 5 Amp, about 6 Amp, about 7 Amp, about 8 Amp, or about 9 Amp. The devices and systems can operate at a current of at most about 2 Amp, about 3 Amp, about 4 Amp, about 5 Amp, about 6 Amp, about 7 Amp, about 8 Amp, about 9 Amp, or about 10 Amp.

The devices and systems can operate at a current of from about 10 Amp to about 25 Amp. The devices and systems can operate at a current of from about 10 Amp to about 12 Amp, about 10 Amp to about 14 Amp, about 10 Amp to about 16 Amp, about 10 Amp to about 18 Amp, about 10 Amp to about 20 Amp, about 10 Amp to about 22 Amp, about 10 Amp to about 25 Amp, about 12 Amp to about 14 Amp, about 12 Amp to about 16 Amp, about 12 Amp to about 18 Amp, about 12 Amp to about 20 Amp, about 12 Amp to about 22 Amp, about 12 Amp to about 25 Amp, about 14 Amp to about 16 Amp, about 14 Amp to about 18 Amp, about 14 Amp to about 20 Amp, about 14 Amp to about 22 Amp, about 14 Amp to about 25 Amp, about 16 Amp to about 18 Amp, about 16 Amp to about 20 Amp, about 16 Amp to about 22 Amp, about 16 Amp to about 25 Amp, about 18 Amp to about 20 Amp, about 18 Amp to about 22 Amp, about 18 Amp to about 25 Amp, about 20 Amp to about 22 Amp, about 20 Amp to about 25 Amp, or about 22 Amp to about 25 Amp. The devices and systems can operate at a current of about 10 Amp, about 12 Amp, about 14 Amp, about 16 Amp, about 18 Amp, about 20 Amp, about 22 Amp, or about 25 Amp. The devices and systems can operate at a current of at least about 10 Amp, about 12 Amp, about 14 Amp, about 16 Amp, about 18 Amp, about 20 Amp, or about 22 Amp. The devices and systems can operate at a current of at most about 12 Amp, about 14 Amp, about 16 Amp, about 18 Amp, about 20 Amp, about 22 Amp, or about 25 Amp.

### Control Circuitry and Pulse Delivery

The devices described herein can comprise a control unit. The control unit can govern the delivery of ultrashort electrical pulses to the cutting element. The control unit can comprise electronics for shaping and delivering pulses.

In some cases, the devices, via instructions from the control unit, can have a pulse shape. The pulse shape can comprise a square wave. The pulse shape can have exponential decay. The waveform may ramp up. The waveform may be modulated.

In some cases, the devices, via instructions from the control unit, can have a pulse duration (e.g., pulse width). The duration (e.g., width) can range from microseconds (e.g., 10-100 us) to milliseconds (1-500 ms).

The pulse duration (e.g., pulse width) can be from about 10 μs to about 100 μs. The pulse duration can be from about 10 μs to about 20 μs, about 10 μs to about 30 μs, about 10 μs to about 40 μs, about 10 μs to about 50 μs, about 10 μs to about 60 μs, about 10 μs to about 70 μs, about 10 μs to about 80 μs, about 10 μs to about 90 μs, about 10 μs to about 100 μs, about 20 μs to about 30 μs, about 20 μs to about 40 μs, about 20 μs to about 50 μs, about 20 μs to about 60 μs, about 20 μs to about 70 μs, about 20 μs to about 80 μs, about 20 μs to about 90 μs, about 20 μs to about 100 μs, about 30 μs to about 40 μs, about 30 μs to about 50 μs, about 30 μs to about 60 μs, about 30 μs to about 70 μs, about 30 μs to about 80 μs, about 30 μs to about 90 μs, about 30 μs to about 100 μs, about 40 μs to about 50 μs, about 40 μs to about 60 μs, about 40 μs to about 70 μs, about 40 μs to about 80 μs, about 40 μs to about 90 μs, about 40 μs to about 100 μs, about 50 μs to about 60 μs, about 50 μs to about 70 μs, about 50 μs to about 80 μs, about 50 μs to about 90 μs, about 50 μs to about 100 μs, about 60 μs to about 70 μs, about 60 μs to about 80 μs, about 60 μs to about 90 μs, about 60 μs to about 100 μs, about 70 μs to about 80 μs, about 70 μs to about 90 μs, about 70 μs to about 100 μs, about 80 μs to about 90 μs, about 80 μs to about 100 μs, or about 90 μs to about 100 μs. The pulse duration can be about 10 μs, about 20 μs, about 30 μs, about 40 μs, about 50 μs, about 60 μs, about 70 μs, about 80 μs, about 90 μs, or about 100 μs. The pulse duration can be at least about 10 μs, about 20 μs, about 30 μs, about 40 μs, about 50 μs, about 60 μs, about 70 μs, about 80 μs, or about 90 μs. The pulse duration can be at most about 20 μs, about 30 μs, about 40 μs, about 50 μs, about 60 μs, about 70 μs, about 80 μs, about 90 μs, or about 100 μs.

The pulse duration (e.g., pulse width) can be from about 1 ms to about 50 ms. The pulse duration can be from about 1 ms to about 5 ms, about 1 ms to about 10 ms, about 1 ms to about 20 ms, about 1 ms to about 30 ms, about 1 ms to about 40 ms, about 1 ms to about 50 ms, about 5 ms to about 10 ms, about 5 ms to about 20 ms, about 5 ms to about 30 ms, about 5 ms to about 40 ms, about 5 ms to about 50 ms, about 10 ms to about 20 ms, about 10 ms to about 30 ms, about 10 ms to about 40 ms, about 10 ms to about 50 ms, about 20 ms to about 30 ms, about 20 ms to about 40 ms, about 20 ms to about 50 ms, about 30 ms to about 40 ms, about 30 ms to about 50 ms, or about 40 ms to about 50 ms. The pulse duration can be about 1 ms, about 5 ms, about 10 ms, about 20 ms, about 30 ms, about 40 ms, or about 50 ms. The pulse duration can be at least about 1 ms, about 5 ms, about 10 ms, about 20 ms, about 30 ms, or about 40 ms. The pulse duration can be at most about 5 ms, about 10 ms, about 20 ms, about 30 ms, about 40 ms, or about 50 ms.

The pulse duration (e.g., pulse width) can be from about 0.001 ms to about 100 ms. The pulse duration can be from about 0.001 ms to about 0.01 ms, about 0.001 ms to about 0.1 ms, about 0.001 ms to about 1 ms, about 0.001 ms to about 10 ms, about 0.001 ms to about 25 ms, about 0.001 ms to about 50 ms, about 0.001 ms to about 75 ms, about 0.001 ms to about 100 ms, about 0.01 ms to about 0.1 ms, about 0.01 ms to about 1 ms, about 0.01 ms to about 10 ms, about 0.01 ms to about 25 ms, about 0.01 ms to about 50 ms, about 0.01 ms to about 75 ms, about 0.01 ms to about 100 ms, about 0.1 ms to about 1 ms, about 0.1 ms to about 10 ms, about 0.1 ms to about 25 ms, about 0.1 ms to about 50 ms, about 0.1 ms to about 75 ms, about 0.1 ms to about 100 ms, about 1 ms to about 10 ms, about 1 ms to about 25 ms, about 1 ms to about 50 ms, about 1 ms to about 75 ms, about 1 ms to about 100 ms, about 10 ms to about 25 ms, about 10 ms to about 50 ms, about 10 ms to about 75 ms, about 10 ms to about 100 ms, about 25 ms to about 50 ms, about 25 ms to about 75 ms, about 25 ms to about 100 ms, about 50 ms to about 75 ms, about 50 ms to about 100 ms, or about 75 ms to about 100 ms. The pulse duration can be about 0.001 ms, about 0.01 ms, about 0.1 ms, about 1 ms, about 10 ms, about 25 ms, about 50 ms, about 75 ms, or about 100 ms. The pulse duration can be at least about 0.001 ms, about 0.01 ms, about 0.1 ms, about 1 ms, about 10 ms, about 25 ms, about 50 ms, or about 75 ms. The pulse duration can be at most about 0.01 ms, about 0.1 ms, about 1 ms, about 10 ms, about 25 ms, about 50 ms, about 75 ms, or about 100 ms.

The pulse duration (e.g., pulse width) can be from about 10 ms to about 150 ms. The pulse duration can be from about 20 ms to about 40 ms, about 20 ms to about 60 ms, about 20 ms to about 80 ms, about 20 ms to about 10 ms, about 20 ms to about 120 ms, about 20 ms to about 150 ms, about 40 ms to about 60 ms, about 40 ms to about 80 ms, about 40 ms to about 10 ms, about 40 ms to about 120 ms, about 40 ms to about 150 ms, about 60 ms to about 80 ms, about 60 ms to about 10 ms, about 60 ms to about 120 ms, about 60 ms to about 150 ms, about 80 ms to about 10 ms, about 80 ms to about 120 ms, about 80 ms to about 150 ms, about 10 ms to about 120 ms, about 10 ms to about 150 ms, or about 120 ms to about 150 ms. The pulse duration can be about 20 ms, about 40 ms, about 60 ms, about 80 ms, about 10 ms, about 120 ms, or about 150 ms. The pulse duration can be at least about 20 ms, about 40 ms, about 60 ms, about 80 ms, about 10 ms, or about 120 ms. The pulse duration can be at most about 40 ms, about 60 ms, about 80 ms, about 10 ms, about 120 ms, or about 150 ms. The pulse duration can be from about 30 ms to about 120 ms.

The pulse duration (e.g., pulse width) can be about 50 ms to about 500 ms. The pulse duration can be about 50 ms to about 100 ms, about 50 ms to about 150 ms, about 50 ms to about 200 ms, about 50 ms to about 250 ms, about 50 ms to about 300 ms, about 50 ms to about 350 ms, about 50 ms to about 400 ms, about 50 ms to about 450 ms, about 50 ms to about 500 ms, about 100 ms to about 150 ms, about 100 ms to about 200 ms, about 100 ms to about 250 ms, about 100 ms to about 300 ms, about 100 ms to about 350 ms, about 100 ms to about 400 ms, about 100 ms to about 450 ms, about 100 ms to about 500 ms, about 150 ms to about 200 ms, about 150 ms to about 250 ms, about 150 ms to about 300 ms, about 150 ms to about 350 ms, about 150 ms to about 400 ms, about 150 ms to about 450 ms, about 150 ms to about 500 ms, about 200 ms to about 250 ms, about 200 ms to about 300 ms, about 200 ms to about 350 ms, about 200 ms to about 400 ms, about 200 ms to about 450 ms, about 200 ms to about 500 ms, about 250 ms to about 300 ms, about 250 ms to about 350 ms, about 250 ms to about 400 ms, about 250 ms to about 450 ms, about 250 ms to about 500 ms, about 300 ms to about 350 ms, about 300 ms to about 400 ms, about 300 ms to about 450 ms, about 300 ms to about 500 ms, about 350 ms to about 400 ms, about 350 ms to about 450 ms, about 350 ms to about 500 ms, about 400 ms to about 450 ms, about 400 ms to about 500 ms, or about 450 ms to about 500 ms. The pulse duration can be about 50 ms, about 100 ms, about 150 ms, about 200 ms, about 250 ms, about 300 ms, about 350 ms, about 400 ms, about 450 ms, or about 500 ms. The pulse duration can be at least about 50 ms, about 100 ms, about 150 ms, about 200 ms, about 250 ms, about 300 ms, about 350 ms, about 400 ms, or about 450 ms. The pulse duration can be at most about 100 ms, about 150 ms, about 200 ms, about 250 ms, about 300 ms, about 350 ms, about 400 ms, about 450 ms, or about 500 ms.

In some cases, the devices, via instructions from the control unit, can have a pulse count. The pulse can comprise a single-shot activation or rapid train of sub-pulses.

The pulses can be triggered in a variety of ways. The trigger can be manual (e.g., via foot pedal, finger button, etc.), automated (e.g., contact sensors), voice-activated, or any combination thereof.

Feedback can be integrated in a variety of ways. The devices can include real-time resistance monitoring to confirm tissue contact. The devices can include current sensors, voltage sensors, or both to terminate the pulse when the circuit is complete. These feedback variations can allow for adaptability across patient anatomies and capsule properties.

### Cutting/Thermal Element

The cutting element can be characterized by an ultralight, ultra-low thermal mass and shape memory conductive loop. The cutting element can also be referred to as a “heating element” or “thermal element” as described above.

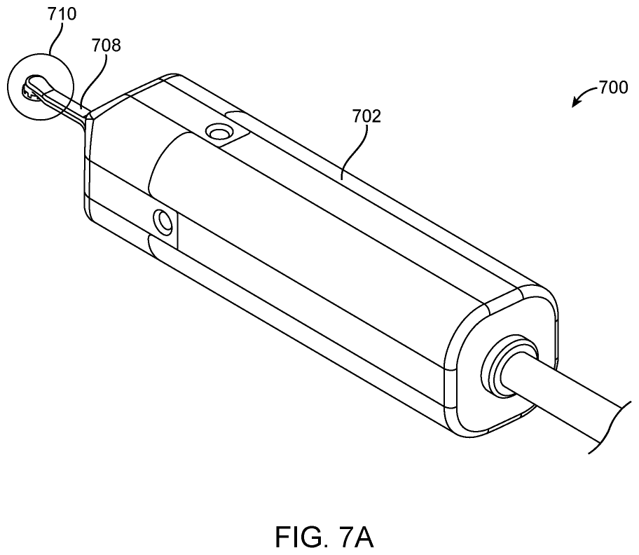

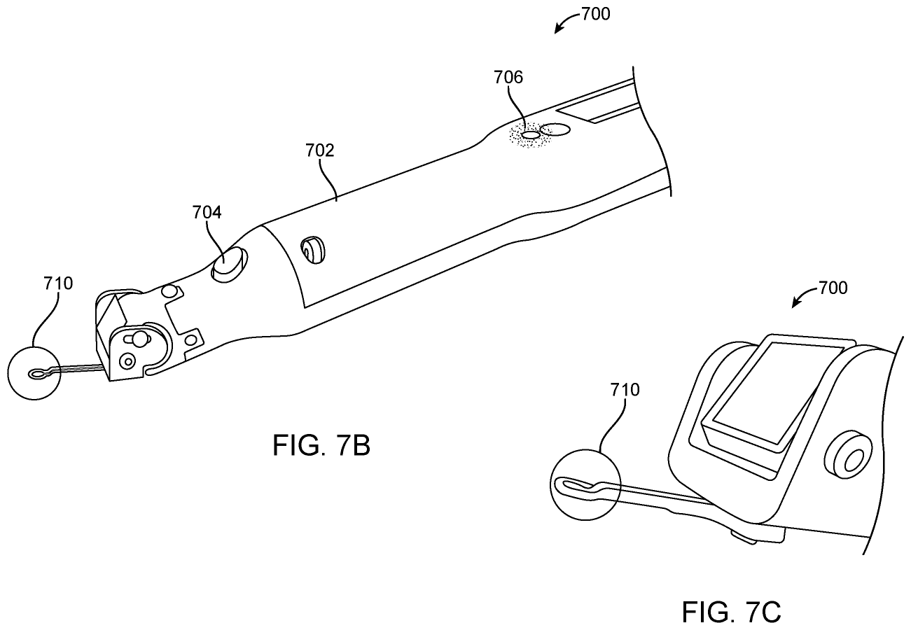

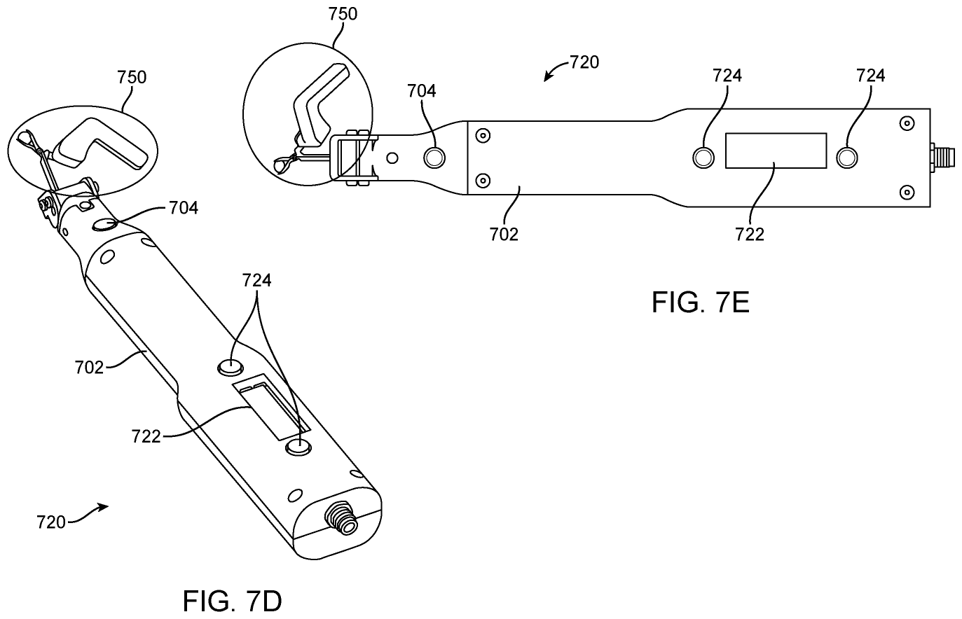

FIGS. 7A-7C show perspective views of a capsulorhexis device 700 comprising a housing 702, a button 704, a charge status light 706, an extended distal section 708, and a distal end 710. In some cases, the housing 702 can comprise the control unit described above. In some cases, the housing 702 can comprise the internal powering features described above. In some cases, the powering features can be external. The distal end 710 can comprise the cutting or thermal element. In some cases, as shown in FIG. 7A, the housing may be unified with the extended distal part 708 of the device 700. In some cases, as shown in FIGS. 7B-7C, the housing may be coupled to but not unified with the extended distal part 708 of the device 700 to allow for movement (e.g., oscillation, rotation, arcing, etc.) of the extended distal part relative to the housing 702. In some cases, the extended distal part 708 can allow for angling or gimballing of the tip relative to the housing. The button 704 can comprise the trigger for the pulse or electricity to travel through the wires (described below) and into the cutting element, thereby cutting the capsular bag. The housing can also be considered the handpiece.

FIGS. 7D-7E show a device 720 comprising a housing/handpiece 702, an actuator button 704, a display 722, parameter buttons 724, and a distal end 750. FIG. 7F shows an exploded view of the handpiece 702 comprising a lower housing 726, an upper housing 728, a printed circuit board (PCB) 734, the display 722, pivoting arms 730 and 732, the actuator button 704, and the parameter buttons 724. The upper and lower housings can enclose the internal elements of the handpiece 702. The distal end 750 can comprise a delivery device and a heating/cutting element as described herein. In some cases, the distal end 750 can allow for angling or gimballing of the tip relative to the housing. Various delivery devices are described below. The PCB 734 can comprise the control unit described herein. The battery can be adjacent the PCB 734. The battery can allow the devices 720 and 700 to be portable. The battery can allow for use of direct current power. The actuator button 704 can comprise the trigger for the pulse or electricity to travel through the wires (described below) and into the cutting element, thereby cutting the capsular bag. The parameter buttons 724 can be used to adjust the parameters of the pulse before and/or during pulse administration. Parameters can include, but are not limited to, one or more of pulse amplitude, duration/width, current, or voltage. In some cases, the display 722 can be used to show a status of the device (e.g., charged/ready, in contact with the capsular bag, etc.). In some cases, the display 722 can be electronically coupled to a distal camera to show the eye and capsular bag that the device is facing. In some cases, the display 722 can show programmed pulse parameters that can be edited and updated in real time.

The distal end of the handpiece 702 can comprise a pivoting arm between the distal end 750 comprising the delivery device and the handpiece 702. The pivoting arm can comprise a proximal section 730 and a distal part 732. The pivoting arm can allow for adjustment of the angle of entry. In some cases, the pivoting arm can be used instead of a gimbal on the cutting element/delivery device tip. In some cases, the pivoting arm can be used in conjunction with the gimbal for additional degrees of angle adjustment.

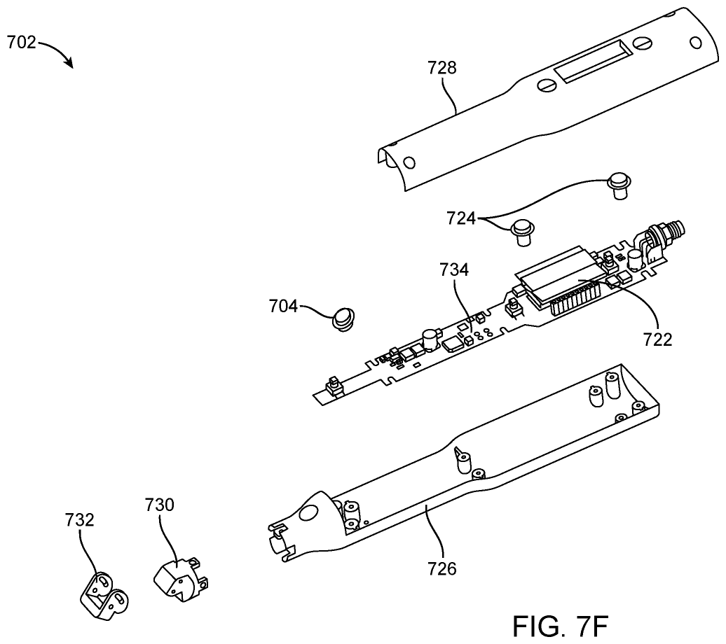

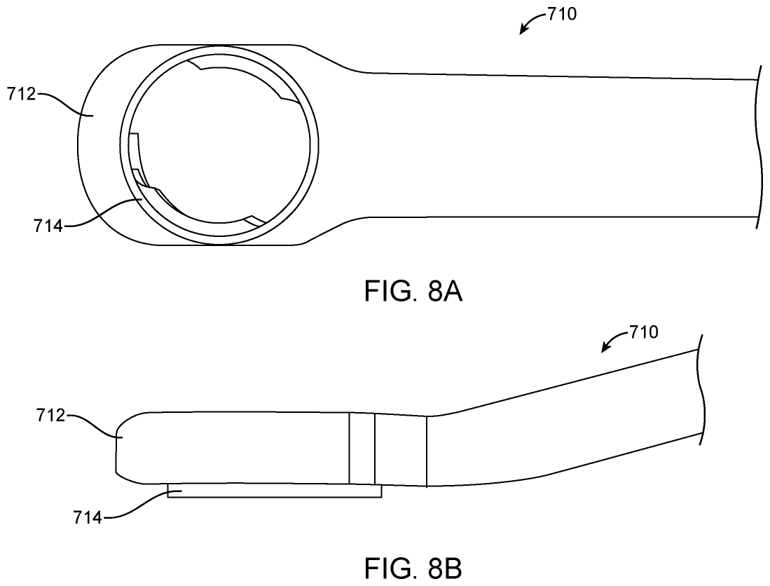

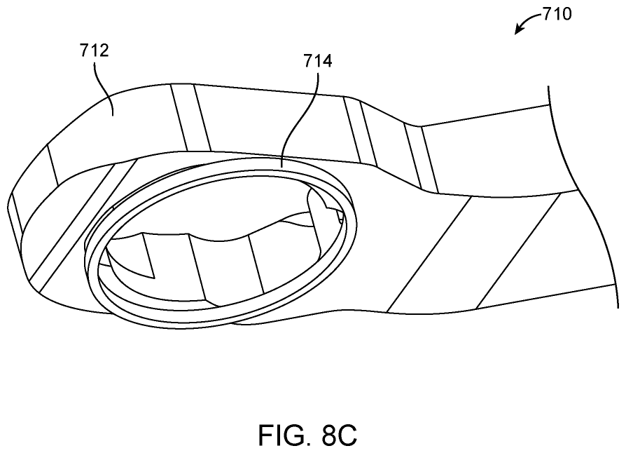

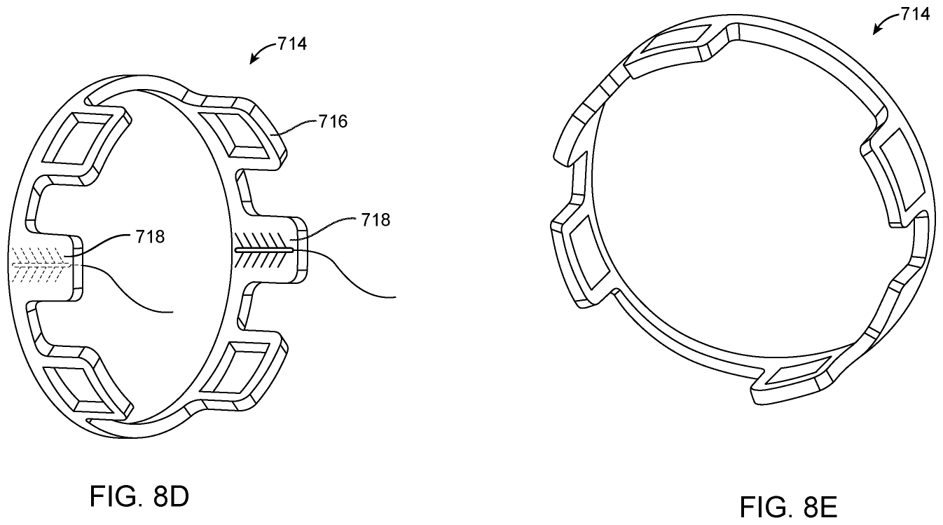

FIGS. 8A-8C show different views of a distal end 710 comprising a tip 712 and a cutting element 714. The tip 712 can be an elongated and thinner part of the distal end 710 of the housing. The tip 712 can be sized to be inserted into an incision of an eye to bring the cutting element 714 near the capsular bag. The cutting element 714 can be an elliptical or circular loop or ring as described above. The cross-section of the cutting element 714 can be rounded or rectangular. In some cases, the tip 712 can comprise a plastic or metal material. In some cases, the tip 712 can comprise a heat-tolerant material. In some cases, the tip 712 can comprise a high-temperature plastic nitinol ring holder.

FIGS. 8D-8E show views of the cutting element 714 comprising snap features 716 and a wire 718. In some cases, the cutting element 714 can comprise the thermal elements described in FIGS. 1-6. Whereas FIGS. 8A-8C may show the parts of the cutting element protruding from the tip of the insertion device, FIGS. 8D-8E may show the other side of the cutting element where it connects to the insertion device.

One or more snap features 716 can attach the cutting element to the distal tip, or a to plastic or metal holder of the distal tip. In some cases, the cutting element can comprise 1, 2, 3, 4, 5, 6, 7, 8, 9, 10 or more snap features. The number of snap features may depend on the type of delivery device. In some cases, the cutting element has no snap features. The snap features may be solid. The snap features may have an empty space that is enclosed by Nitinol or another material. In some cases, the snap features can be symmetrical. In some cases, the snap features may be asymmetrical. In some cases, rather than snapping into a delivery device, the cutting element may have holes where connective wires can be threaded into from the delivery device during delivery.

In some cases, a wire 718 can be laser welded to the nitinol ring comprising the cutting element. In some cases, the wire can be welded to another protrusion of the cutting element. In some cases, the protrusion may be solid without holes. In some cases, the protrusion may have one or more holes. In some cases, the cutting element can comprise 1, 2, 3, 4, 5, 6, 7, 8, 9, 10 or more protrusions for laser welding. In some cases, the protrusions can be symmetrical and have the wire welded to opposite protrusions. In some cases, the protrusions may be asymmetrical. In some cases, depending on the delivery device, there may not be a wire 718 laser welded to the cutting element.

The cutting element may comprise nitinol (e.g., a nickel-titanium alloy). In some cases, the cutting element may comprise a 2.8 mm OD nitinol ring laser cut out of a hypotube. Nitinol can be beneficial for flexibility, shape memory, and rapid thermal response.

In some cases, the cutting element may comprise platinum, iridium, nitinol, tungsten, molybdenum, stainless steel, cobalt-chrome, composite conductors, or any combination thereof. In some cases, the cutting element may comprise platinum-iridium, which has high conductivity and biocompatibility. Tungsten or molybdenum may comprise high melting point alloys. Stainless steel can comprise a low-cost alternative. Composite conductors can comprise carbon nanotube bundles or conductive polymers.

The cutting element may have a cross section. The cutting element may comprise a wire having a round cross-section with a diameter from about 25 μm to about 100 μm. The cutting element can comprise a flat ribbon for more uniform thermal distribution. The cutting element can comprise braided microfilaments for redundancy. The cutting element can comprise hollow-core tubes for ultralight mass. The cutting element can comprise laser-cut tubes for ultralight mass.

The cutting element may have a treated surface. In some cases, the treated surface can include insulation. The insulation can comprise parylene, silicone, or polymer coatings on non-cutting sections. In some cases, the treated surface can include surface texturing. The surface texturing can comprise micro-serrations or etched surfaces for mechanical anchoring. In some cases, the treated surface can include coatings, including but not limited to hydrophobic polytetrafluoroethylene (PTFE) for smooth tissue interaction.

The cutting element or cutting loop can take many shapes to enable different capsulotomy designs. For a standard capsulotomy, the cutting element may have a substantially circular shape with a diameter from about 1 mm to about 7 mm. For astigmatism-correcting IOLs, the cutting element may be elliptical. To mimic manual CCC outcomes, the cutting element may be polygonal (hexagon, octagon). For experimental optical effects, the cutting element may be petal-shaped or lobed. Such shapes can be helpful for creating a desired distortion to light entering the lens before hitting the retina. For selective capsule opening, the cutting element may comprise partial arcs or semicircles. Multi-loop concentric designs may be used for double-ring openings. Each geometry can be pre-formed or adjustable intraoperatively.

The cutting element may have various deployment mechanisms, based in part on the insertion device. The cutting element may be foldable, such that it can be compressible into a tubular sheath for an incision no greater than about 2 mm. The cutting element may have a shape-memory expansion feature. A nitinol loop can self-deploy into a preset geometry when released. There can be a sliding tube design, such that the cutting element can be retracted into a cannula, then extended. There can be a spring-loaded mechanism, such that the cutting element can snap into shape inside an anterior chamber. There can be a cartridge-based system with disposable preloaded loops for sterile use.

### Safety Features

The devices described herein may have various safety features. The safety features can comprise feedback components, including but not limited to sensors, interlocks, and control logic. The devices can include thermal sensors comprising miniature thermocouples near the cutting element to monitor a temperature of the cutting element. The devices can include a current cutoff that can detect completion of the capsule cut and stop the pulse. The devices can include contact sensors that can prevent activation unless the cutting element is in the correct eye chamber. The devices can include insulated non-contact regions of the device and/or the cutting loop to prevent or minimize injury to the iris, cornea, and other structures of the eye. The devices can include a smart controller that terminates energy if the resistance drops suddenly, suggesting a misfire.

### Gimballed Contact Frame

Energy-based capsulotomy systems can be used to improve reproducibility. These energy-based systems may rely on a ring-like cutting element that is intended to sit against the anterior capsule while energy is applied around the perimeter of the ring-like cutting element. The cutting element can have a closed perimeter.

However, such devices may face various limitations. Ensuring uniform contact between the cutting element and the capsular bag may be difficult. The cutting element can be fixed in orientation relative to a handpiece or housing. If the lens is tilted or the approach angle is not perfectly orthogonal, some segments of the element may not contact the capsule firmly, leading to incomplete cuts or tags. Some systems may rely on large consoles, complex user interfaces, or external fixation hardware. The reliance on such infrastructure can increase cost and footprint and may use dedicated space and setup time.

Limited adaptability to different anatomies and workflows can be a concern. Many designs may not integrate easily with robotic arms, or such designs may use specialized setup that complicates adoption in high-volume or resource-limited settings.

Accordingly, a need exists for a compact, direct-contact ultrashort-pulse capsulotomy system that can maintain substantially uniform contact with the capsule across variations in lens tilt and eye anatomy; can achieve uniform contact through mechanical self-alignment rather than complex external infrastructure; can be combined with disposable, customizable distal tips to support multiple sizes and shapes of capsulotomy; can be compatible with robotic arms; and can operate from a portable, battery-powered controller rather than a large console.

The present disclosure relates to ophthalmic surgical instruments and methods. More particularly, the present disclosure may concern devices and techniques for creating an opening in the lens capsule during cataract surgery using a direct-contact ultrashort-pulse ablation element mounted on a gimballed contact frame, with optional use on robotic arms, disposable and customizable distal tips, and a portable battery-powered controller.

Provided herein are devices, systems, and methods addressing the above needs by providing a gimballed contact frame at the distal end of an ophthalmic instrument (manual or robotic) that carries a closed-loop conductor defining the capsulotomy contour. The contact frame can be connected to the instrument by one or more joint structures that allow tilting in multiple directions, similar in function to a miniature gimbal. When the surgeon or robotic arm advances the instrument so that the frame reaches the anterior capsule, the frame self-aligns with the capsule surface and can seat along its entire perimeter.

The devices, systems, and methods disclosed herein can provide uniform contact without complex infrastructure. The devices can comprise a gimballed contact frame that can mechanically conform to the capsule of the eye, promoting full perimeter contact without reliance on large external support equipment. The devices can allow for high precision and reproducibility, as the capsulotomy geometry can be dictated by the frame contour, making outcomes less dependent on manual tearing skills. The handpiece of the devices disclosed herein can be used by a surgeon or mounted to a robotic arm, and the same core technology can support both workflows. Disposable, customizable tips and a tip-based design can simplify sterilization, enable rapid changeover between different diameters and shapes, and support high-volume practice. The battery-powered controller may remove the need for a large console. The system can be portable, have a small footprint and be suitable for standard operating rooms, ambulatory centers, and outreach settings. The devices disclosed herein can be low energy and have a localized effect. The low-mass conductor and ultrashort pulse approach can confine energy to the targeted capsule area, preserving neighboring ocular structures.

By combining a gimballed contact frame, disposability, customizable geometry, and a portable controller, the disclosure herein can deliver a capsulotomy solution that is precise, reproducible, infrastructure-light, and that can be used manually or as part of a robotically controlled cataract surgery workflow.

The devices and systems disclosed herein can comprise a portable controller, a handpiece (which may be held by a surgeon or mounted on a robotic arm), and a distal contact assembly that houses the gimballed contact frame and closed-loop conductor. One or more electrical leads can run from the controller through the handpiece to the assembly. The controller can generate ultrashort pulses at controlled voltage and current levels to energize the conductor at the distal end.

### Contact Frame and Gimballed Connection

### Contact Frame Geometry

The distal assembly can comprise a contact frame that defines a perimeter around which the capsule is to be cut. The contact frame can be part of a removable distal tip that attaches to a reusable handle or a robotic adapter. The “contact frame,” “frame,” “cutting element,” “heating element,” and “thermal element” can refer to similar elements as described herein.

The frame can be a thin, ring-like element formed from a biocompatible material such as stainless steel, titanium, nitinol, or a polymer. A longer description of potential materials can be found above. The frame may define a substantially circular path when deployed, but other shapes such as ellipses, polygons, or customized curves may be used as described above. The contact frame can have a shape-memory geometry. The frame can have a closed perimeter. The contact frame can be flexible enough to be foldable into a tubular or elliptical configuration for insertion into an eye of a subject.

Representative perimeter sizes can comprise about 0.1 mm to about 10 mm for overall range; about 5 mm to about 6 mm for standard adult capsulotomy; and about 2 mm to about 3 mm for pediatric or specialized cases.

The frame perimeter can be from about 0.1 mm to about 10 mm. The frame perimeter can be from about 0.1 mm to about 0.5 mm, about 0.1 mm to about 1 mm, about 0.1 mm to about 2 mm, about 0.1 mm to about 3 mm, about 0.1 mm to about 4 mm, about 0.1 mm to about 5 mm, about 0.1 mm to about 6 mm, about 0.1 mm to about 7 mm, about 0.1 mm to about 8 mm, about 0.1 mm to about 9 mm, about 0.1 mm to about 10 mm, about 0.5 mm to about 1 mm, about 0.5 mm to about 2 mm, about 0.5 mm to about 3 mm, about 0.5 mm to about 4 mm, about 0.5 mm to about 5 mm, about 0.5 mm to about 6 mm, about 0.5 mm to about 7 mm, about 0.5 mm to about 8 mm, about 0.5 mm to about 9 mm, about 0.5 mm to about 10 mm, about 1 mm to about 2 mm, about 1 mm to about 3 mm, about 1 mm to about 4 mm, about 1 mm to about 5 mm, about 1 mm to about 6 mm, about 1 mm to about 7 mm, about 1 mm to about 8 mm, about 1 mm to about 9 mm, about 1 mm to about 10 mm, about 2 mm to about 3 mm, about 2 mm to about 4 mm, about 2 mm to about 5 mm, about 2 mm to about 6 mm, about 2 mm to about 7 mm, about 2 mm to about 8 mm, about 2 mm to about 9 mm, about 2 mm to about 10 mm, about 3 mm to about 4 mm, about 3 mm to about 5 mm, about 3 mm to about 6 mm, about 3 mm to about 7 mm, about 3 mm to about 8 mm, about 3 mm to about 9 mm, about 3 mm to about 10 mm, about 4 mm to about 5 mm, about 4 mm to about 6 mm, about 4 mm to about 7 mm, about 4 mm to about 8 mm, about 4 mm to about 9 mm, about 4 mm to about 10 mm, about 5 mm to about 6 mm, about 5 mm to about 7 mm, about 5 mm to about 8 mm, about 5 mm to about 9 mm, about 5 mm to about 10 mm, about 6 mm to about 7 mm, about 6 mm to about 8 mm, about 6 mm to about 9 mm, about 6 mm to about 10 mm, about 7 mm to about 8 mm, about 7 mm to about 9 mm, about 7 mm to about 10 mm, about 8 mm to about 9 mm, about 8 mm to about 10 mm, or about 9 mm to about 10 mm. The frame perimeter can be about 0.1 mm, about 0.5 mm, about 1 mm, about 2 mm, about 3 mm, about 4 mm, about 5 mm, about 6 mm, about 7 mm, about 8 mm, about 9 mm, or about 10 mm. The frame perimeter can be at least about 0.1 mm, about 0.5 mm, about 1 mm, about 2 mm, about 3 mm, about 4 mm, about 5 mm, about 6 mm, about 7 mm, about 8 mm, or about 9 mm. The frame perimeter can be at most about 0.5 mm, about 1 mm, about 2 mm, about 3 mm, about 4 mm, about 5 mm, about 6 mm, about 7 mm, about 8 mm, about 9 mm, or about 10 mm.

A closed-loop conductor-for example, a low-mass metallic wire, strip, or deposited layer—can be integrated with or mounted on the frame. The conductor can follow the path defined by the perimeter and can be electrically connected to leads that run back to the controller. In some cases, the closed-loop conductor can comprise wires.

The conductor cross-section can be chosen to provide low thermal mass and efficient Ohmic heating in response to DC or AC pulses. In some cases, low thermal mass as described herein can comprise a conductor mass (comprising the wires and the contact frame) from about 1 mg to about 20 mg. The thermal mass can be from about 1 mg to about 20 mg. The thermal mass can be from about 1 mg to about 2 mg, about 1 mg to about 4 mg, about 1 mg to about 6 mg, about 1 mg to about 8 mg, about 1 mg to about 10 mg, about 1 mg to about 12 mg, about 1 mg to about 14 mg, about 1 mg to about 16 mg, about 1 mg to about 18 mg, about 1 mg to about 20 mg, about 2 mg to about 4 mg, about 2 mg to about 6 mg, about 2 mg to about 8 mg, about 2 mg to about 10 mg, about 2 mg to about 12 mg, about 2 mg to about 14 mg, about 2 mg to about 16 mg, about 2 mg to about 18 mg, about 2 mg to about 20 mg, about 4 mg to about 6 mg, about 4 mg to about 8 mg, about 4 mg to about 10 mg, about 4 mg to about 12 mg, about 4 mg to about 14 mg, about 4 mg to about 16 mg, about 4 mg to about 18 mg, about 4 mg to about 20 mg, about 6 mg to about 8 mg, about 6 mg to about 10 mg, about 6 mg to about 12 mg, about 6 mg to about 14 mg, about 6 mg to about 16 mg, about 6 mg to about 18 mg, about 6 mg to about 20 mg, about 8 mg to about 10 mg, about 8 mg to about 12 mg, about 8 mg to about 14 mg, about 8 mg to about 16 mg, about 8 mg to about 18 mg, about 8 mg to about 20 mg, about 10 mg to about 12 mg, about 10 mg to about 14 mg, about 10 mg to about 16 mg, about 10 mg to about 18 mg, about 10 mg to about 20 mg, about 12 mg to about 14 mg, about 12 mg to about 16 mg, about 12 mg to about 18 mg, about 12 mg to about 20 mg, about 14 mg to about 16 mg, about 14 mg to about 18 mg, about 14 mg to about 20 mg, about 16 mg to about 18 mg, about 16 mg to about 20 mg, or about 18 mg to about 20 mg. The thermal mass can be about 1 mg, about 2 mg, about 4 mg, about 6 mg, about 8 mg, about 10 mg, about 12 mg, about 14 mg, about 16 mg, about 18 mg, or about 20 mg. The thermal mass can be at least about 1 mg, about 2 mg, about 4 mg, about 6 mg, about 8 mg, about 10 mg, about 12 mg, about 14 mg, about 16 mg, or about 18 mg. The thermal mass can be at most about 2 mg, about 4 mg, about 6 mg, about 8 mg, about 10 mg, about 12 mg, about 14 mg, about 16 mg, about 18 mg, or about 20 mg.

Example cross-sections may be in the range of tens of microns. The cross-sectional size may be from about 0.01 mm to about 0.5 mm. The cross-sectional size may be from about 0.01 mm to about 0.02 mm, about 0.01 mm to about 0.04 mm, about 0.01 mm to about 0.06 mm, about 0.01 mm to about 0.08 mm, about 0.01 mm to about 0.1 mm, about 0.01 mm to about 0.125 mm, about 0.01 mm to about 0.15 mm, about 0.01 mm to about 0.2 mm, about 0.01 mm to about 0.3 mm, about 0.01 mm to about 0.4 mm, about 0.01 mm to about 0.5 mm, about 0.02 mm to about 0.04 mm, about 0.02 mm to about 0.06 mm, about 0.02 mm to about 0.08 mm, about 0.02 mm to about 0.1 mm, about 0.02 mm to about 0.125 mm, about 0.02 mm to about 0.15 mm, about 0.02 mm to about 0.2 mm, about 0.02 mm to about 0.3 mm, about 0.02 mm to about 0.4 mm, about 0.02 mm to about 0.5 mm, about 0.04 mm to about 0.06 mm, about 0.04 mm to about 0.08 mm, about 0.04 mm to about 0.1 mm, about 0.04 mm to about 0.125 mm, about 0.04 mm to about 0.15 mm, about 0.04 mm to about 0.2 mm, about 0.04 mm to about 0.3 mm, about 0.04 mm to about 0.4 mm, about 0.04 mm to about 0.5 mm, about 0.06 mm to about 0.08 mm, about 0.06 mm to about 0.1 mm, about 0.06 mm to about 0.125 mm, about 0.06 mm to about 0.15 mm, about 0.06 mm to about 0.2 mm, about 0.06 mm to about 0.3 mm, about 0.06 mm to about 0.4 mm, about 0.06 mm to about 0.5 mm, about 0.08 mm to about 0.1 mm, about 0.08 mm to about 0.125 mm, about 0.08 mm to about 0.15 mm, about 0.08 mm to about 0.2 mm, about 0.08 mm to about 0.3 mm, about 0.08 mm to about 0.4 mm, about 0.08 mm to about 0.5 mm, about 0.1 mm to about 0.125 mm, about 0.1 mm to about 0.15 mm, about 0.1 mm to about 0.2 mm, about 0.1 mm to about 0.3 mm, about 0.1 mm to about 0.4 mm, about 0.1 mm to about 0.5 mm, about 0.125 mm to about 0.15 mm, about 0.125 mm to about 0.2 mm, about 0.125 mm to about 0.3 mm, about 0.125 mm to about 0.4 mm, about 0.125 mm to about 0.5 mm, about 0.15 mm to about 0.2 mm, about 0.15 mm to about 0.3 mm, about 0.15 mm to about 0.4 mm, about 0.15 mm to about 0.5 mm, about 0.2 mm to about 0.3 mm, about 0.2 mm to about 0.4 mm, about 0.2 mm to about 0.5 mm, about 0.3 mm to about 0.4 mm, about 0.3 mm to about 0.5 mm, or about 0.4 mm to about 0.5 mm. The cross-sectional size may be about 0.01 mm, about 0.02 mm, about 0.04 mm, about 0.06 mm, about 0.08 mm, about 0.1 mm, about 0.125 mm, about 0.15 mm, about 0.2 mm, about 0.3 mm, about 0.4 mm, or about 0.5 mm. The cross-sectional size may be at least about 0.01 mm, about 0.02 mm, about 0.04 mm, about 0.06 mm, about 0.08 mm, about 0.1 mm, about 0.125 mm, about 0.15 mm, about 0.2 mm, about 0.3 mm, or about 0.4 mm. The cross-sectional size may be at most about 0.02 mm, about 0.04 mm, about 0.06 mm, about 0.08 mm, about 0.1 mm, about 0.125 mm, about 0.15 mm, about 0.2 mm, about 0.3 mm, about 0.4 mm, or about 0.5 mm.

### Gimbal Structure

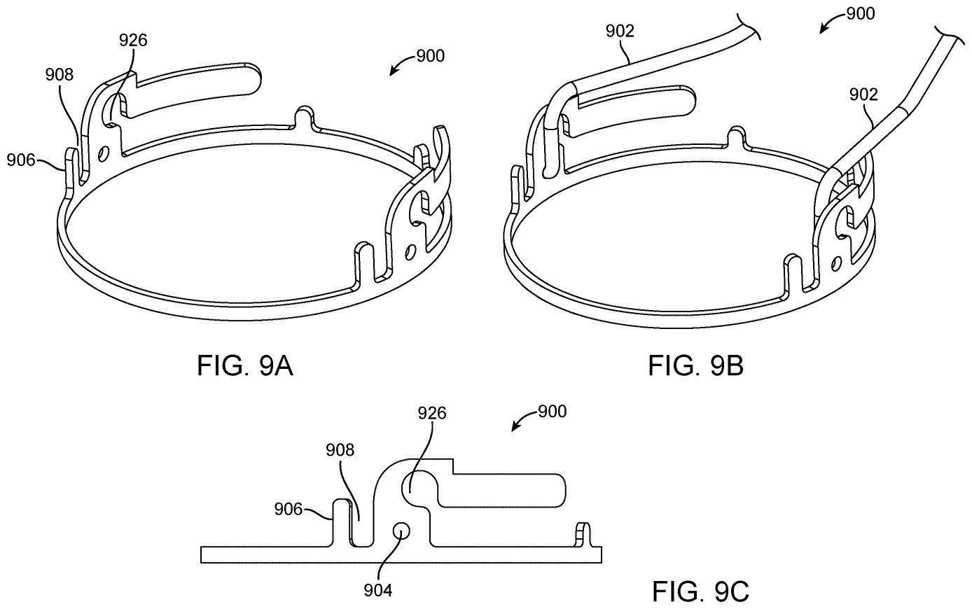

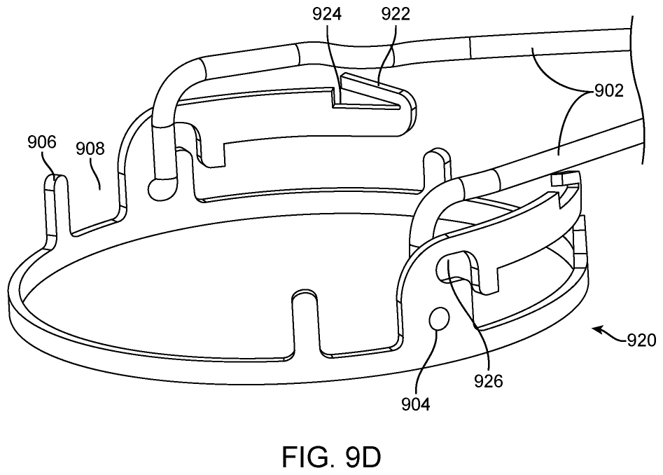

FIGS. 9A-9C show a gimballed contact frame 900 that can be coupled to a wire 902. The wire 902 can comprise a closed-loop conductor as described above. The gimbal can comprise a pivot pin 904 to assist with angling the gimbal upon contact with the capsular bag surface. The gimbal can comprise a slot interface 908 for being held by the delivery device. The slot interface 908 can also act as a limiter on the gimballing ability, as the delivery device slot cannot go lower (and the frame cannot go higher) than where the slot interacts with the frame within the slot interface 908. The gimbal can comprise a gimbal hinge 926. The frame can comprise an upstanding tab 906 with a hole that receives the pivot pin 904 on a support structure. The gimbal can comprise opposed posts and sockets. The tabs or side posts 906 can snap into sockets on support arms extending from the delivery device, allowing limited angular movement.

In some cases, the gimbal hinge 926 can comprise a rounded elbow structure surrounding an open, substantially circular or elliptical area. The open area may have an opened corner or side. The hinge 926 can pivot along an axis to result in perpendicular contact with the capsular bag regardless of handpiece and delivery device insertion angle. The gimbal hinge 926 can comprise a pivoting feature that allows the delivery device to come into the eye at an angle and still have the cutting element be flush with a central axis of the capsular bag.

In some cases, as shown in FIG. 9D, in addition to the above elements, the frame 920 can comprise a hooked segment 922 connected via a thin flexural bridge 924 that may allow limited rotation. The frame 920 can be similar to the frame 900 but with the addition of the hooked segments 922 and the bridge 924. The hooked segments 922 and the bridge 924 can act as anchors in the delivery device, allowing the frame to pivot via elements 908, 904, 906, and 926 while creating a limiter on the amount of angling or rotation. The hooked segments 922 and the bridge 924 can help to keep the frame oriented properly (e.g., without flipping upside down) and can prescribe a limit to otherwise free angling of the contact frame relative to the capsular bag. The limited angling can help a user remain within a prescribed acceptable range for puncturing the capsular bag (e.g., if the gimbal is limited to about 15 degrees and the user moves the device such that the device is at a 90-degree angle relative to the capsular bag, the gimbal may not compensate, and the user can know that the user is outside the desired area for capsulorhexis). In some cases, the pin 904 can assist with pivoting. In some cases, the pin 904 can act as a flexure slot interface and may be optional. In some cases, the primary flexing for the gimbal action can occur via the slot interface 908 and hooked segment 922, while the cutting surface floats freely and can pivot.

The contact frame can be connected to a support structure at the distal end of the handpiece through a gimbal arrangement. The gimbal arrangement can comprise one or more joints that permit tilting of the frame about at least one, two, or three orthogonal axes. In some cases, the joint geometry can comprise one or more of pivot pin 904, slot interface 908, or hooked segment 922. The gimbal arrangement can be implemented in various ways.

The gimbal can comprise an integral flexure. A portion of the frame or support can be thinned and shaped to function as a compliant hinge that may yield when the frame encounters the capsule surface.

In each case, the joint geometry can be selected so that the frame can tilt by a predetermined amount. As a non-limiting example, the predetermined amount can be ±about 10 degrees to about 30 degrees or up to about 90 degrees on any axis. The frame can tilt while maintaining overall dimensional stability of the perimeter. A bias element such as a spring tongue, elastomeric pad, or flexural tab may urge the frame toward a neutral plane when not loaded. In some cases, the joint geometry can comprise one or more of pivot pin 904, slot interface 908, or hooked segment 922.

The gimbal can be pre-set to allow for a tilt of from about 5 degrees to about 90 degrees on any axis. The gimbal can be pre-set to allow for a tilt of from about 5 degrees to about 10 degrees, about 5 degrees to about 15 degrees, about 5 degrees to about 20 degrees, about 5 degrees to about 30 degrees, about 5 degrees to about 40 degrees, about 5 degrees to about 50 degrees, about 5 degrees to about 60 degrees, about 5 degrees to about 70 degrees, about 5 degrees to about 80 degrees, about 5 degrees to about 90 degrees, about 10 degrees to about 15 degrees, about 10 degrees to about 20 degrees, about 10 degrees to about 30 degrees, about 10 degrees to about 40 degrees, about 10 degrees to about 50 degrees, about 10 degrees to about 60 degrees, about 10 degrees to about 70 degrees, about 10 degrees to about 80 degrees, about 10 degrees to about 90 degrees, about 15 degrees to about 20 degrees, about 15 degrees to about 30 degrees, about 15 degrees to about 40 degrees, about 15 degrees to about 50 degrees, about 15 degrees to about 60 degrees, about 15 degrees to about 70 degrees, about 15 degrees to about 80 degrees, about 15 degrees to about 90 degrees, about 20 degrees to about 30 degrees, about 20 degrees to about 40 degrees, about 20 degrees to about 50 degrees, about 20 degrees to about 60 degrees, about 20 degrees to about 70 degrees, about 20 degrees to about 80 degrees, about 20 degrees to about 90 degrees, about 30 degrees to about 40 degrees, about 30 degrees to about 50 degrees, about 30 degrees to about 60 degrees, about 30 degrees to about 70 degrees, about 30 degrees to about 80 degrees, about 30 degrees to about 90 degrees, about 40 degrees to about 50 degrees, about 40 degrees to about 60 degrees, about 40 degrees to about 70 degrees, about 40 degrees to about 80 degrees, about 40 degrees to about 90 degrees, about 50 degrees to about 60 degrees, about 50 degrees to about 70 degrees, about 50 degrees to about 80 degrees, about 50 degrees to about 90 degrees, about 60 degrees to about 70 degrees, about 60 degrees to about 80 degrees, about 60 degrees to about 90 degrees, about 70 degrees to about 80 degrees, about 70 degrees to about 90 degrees, or about 80 degrees to about 90 degrees. The gimbal can allow for a tilt of about 5 degrees, about 10 degrees, about 15 degrees, about 20 degrees, about 30 degrees, about 40 degrees, about 50 degrees, about 60 degrees, about 70 degrees, about 80 degrees, or about 90 degrees. The gimbal can allow for a tilt of at least about 5 degrees, about 10 degrees, about 15 degrees, about 20 degrees, about 30 degrees, about 40 degrees, about 50 degrees, about 60 degrees, about 70 degrees, or about 80 degrees. The gimbal can allow for a tilt of at most about 10 degrees, about 15 degrees, about 20 degrees, about 30 degrees, about 40 degrees, about 50 degrees, about 60 degrees, about 70 degrees, about 80 degrees, or about 90 degrees.

When the surgeon or robotic arm advances the assembly toward the anterior capsule, contact between the capsule and frame can cause the frame to tilt and seat against the capsule surface around its circumference. The gimbal can allow this seating to occur even if the approach angle of the handpiece is not perfectly orthogonal or if the lens is naturally tilted.

### Mechanical Expansion and Retraction

To allow the contact frame to be introduced through a small corneal incision, the distal assembly can have a compact configuration for insertion and a larger configuration for treatment.

The frame can be mechanically reconfigurable between a compact insertion configuration and a deployed configuration using a scissor-like linkage or a tube-based mechanism that expands or moves the frame without the need for external fixation arrangements.

### Scissor-Type Mechanism

In a scissor-like or forceps-like arrangement as discussed above, the contact frame can be mounted near the distal ends of the arms. Movement of a proximal actuator can bring the proximal ends of the arms together or apart, causing the distal ends to move inversely and thereby adjust the effective size or orientation of the frame.

In some cases, when the proximal ends are separated, the distal ends move closer together, drawing the frame into a reduced profile comparable to the shaft diameter for insertion through a small incision. In some cases, when the proximal ends are brought together, the distal ends move apart and the frame assumes the final deployed geometry. The geometry can be based on one or more of a shape memory, a geometry formed by the delivery devices, or one of the varying geometries described above. The gimbal structure can remain operative in both states so that the frame can still tilt and conform to the capsule surface.

### Tube-Based Mechanism

In a tube-based mechanism, a distal tube can surround at least part of the contact frame and support structure. A slider connected to the frame can be moved relative to the tube. In some cases, the tube can be a half-tube or half-pipe at the distal end as described above.

In an insertion state, the frame can be at least partially housed at or just inside the tube's distal opening, yielding a compact profile for passage through the incision. Advancing the slider can move the frame out of the tube and into a deployed configuration with the full treatment perimeter exposed. Retracting the slider can return the frame to the insertion configuration. The tube mechanism can be straight or angled to match the geometry of the handpiece and can be actuated by the surgeon or by a robotic arm.

The geometry can be based on one or more of a shape memory, a geometry formed by the delivery devices, or one of the varying geometries described above.

### Disposable and Customizable Distal Tips

Different tips can provide different perimeter sizes and shapes. Representative perimeter sizes can include about 0.1 mm to about 10 mm for overall range; about 5 mm to about 6 mm for standard adult capsulotomy; and about 2 mm to about 3 mm for pediatric or specialized cases.

In some embodiments, the contact frame, the gimbal arrangement, and at least the distal portion of the support structure can be included in a removable distal tip that attaches to a reusable handle or robotic adapter.

The tip may comprise a mechanical coupling such as a bayonet connector, snap-fit latch, or threaded interface. The tip may comprise electrical contacts that automatically engage leads from the controller when the tip is attached. The tip may comprise integrated frame and conductor components that have been pre-manufactured to a specific geometry and size. The tip may comprise optional color coding, markings, or RFID tags for diameter and shape identification.

A family of tips can be provided with different frame sizes (e.g., about 0.1 mm to about 10 mm) and shapes. Standard sets may include, but are not limited to, 2.5 mm, 2.8 mm, 3.0 mm, 4.0 mm, 5.0 mm, 5.5 mm, and 6.0 mm frames for common indications. Additional tip geometries can be designed for specialized capsule or iris procedures.

After use, the tip can be discarded, and a new sterile tip can be attached for the next case. This can minimize reprocessing, maintain performance of delicate gimbal and conductor components, and simplify integration with robotics (e.g., where tips can be loaded into a magazine for robotic selection).

### Portable Battery-Powered Controller

The devices and systems disclosed herein can comprise a portable controller. The controller can be powered by an internal rechargeable battery. The controller can generate one or more ultrashort pulses of current and deliver them to the closed-loop conductor. Because the conductor has low thermal mass and can be in direct contact with the capsule along the entire perimeter of the frame, the applied energy can rapidly and locally heat the contact region, ablating the capsule along the frame's path. The pulse duration is sufficiently brief that the conductor may cool quickly and energy may not significantly propagate beyond the targeted tissue.

The controller can be compact and self-contained, designed to operate without a large console. The controller can comprise a rechargeable battery pack. The controller can comprise a converter and an energy storage stage (e.g., capacitors). The controller can comprise a pulse generator (e.g., microcontroller, application-specific integrated circuit (ASIC), or field-programmable gate array (FPGA)) that defines pulse amplitude, duration, and waveform. The controller can comprise a user interface (e.g., buttons, touch screen, rotary knob) to allow a user to select parameters or preset profiles. The controller can comprise indicators such as LEDs or a display for status, battery level, and tip recognition.

The energy pulses can have a controlled voltage. The controlled voltage can be from about 0 V to about 240 V. The controlled voltage can be from about 0 V to about 20 V, about 0 V to about 40 V, about 0 V to about 60 V, about 0 V to about 80 V, about 0 V to about 100 V, about 0 V to about 120 V, about 0 V to about 140 V, about 0 V to about 160 V, about 0 V to about 200 V, about 0 V to about 240 V, about 20 V to about 40 V, about 20 V to about 60 V, about 20 V to about 80 V, about 20 V to about 100 V, about 20 V to about 120 V, about 20 V to about 140 V, about 20 V to about 160 V, about 20 V to about 200 V, about 20 V to about 240 V, about 40 V to about 60 V, about 40 V to about 80 V, about 40 V to about 100 V, about 40 V to about 120 V, about 40 V to about 140 V, about 40 V to about 160 V, about 40 V to about 200 V, about 40 V to about 240 V, about 60 V to about 80 V, about 60 V to about 100 V, about 60 V to about 120 V, about 60 V to about 140 V, about 60 V to about 160 V, about 60 V to about 200 V, about 60 V to about 240 V, about 80 V to about 100 V, about 80 V to about 120 V, about 80 V to about 140 V, about 80 V to about 160 V, about 80 V to about 200 V, about 80 V to about 240 V, about 100 V to about 120 V, about 100 V to about 140 V, about 100 V to about 160 V, about 100 V to about 200 V, about 100 V to about 240 V, about 120 V to about 140 V, about 120 V to about 160 V, about 120 V to about 200 V, about 120 V to about 240 V, about 140 V to about 160 V, about 140 V to about 200 V, about 140 V to about 240 V, about 160 V to about 200 V, about 160 V to about 240 V, or about 200 V to about 240 V. The controlled voltage can be about 0 V, about 20 V, about 40 V, about 60 V, about 80 V, about 100 V, about 120 V, about 140 V, about 160 V, about 200 V, or about 240 V. The controlled voltage can be at least about 0 V, about 20 V, about 40 V, about 60 V, about 80 V, about 100 V, about 120 V, about 140 V, about 160 V, or about 200 V. The controlled voltage can be at most about 20 V, about 40 V, about 60 V, about 80 V, about 100 V, about 120 V, about 140 V, about 160 V, about 200 V, or about 240 V.

The controlled voltage can be from about 1 V to about 20 V. The controlled voltage can be from about 1 V to about 2 V, about 1 V to about 4 V, about 1 V to about 6 V, about 1 V to about 8 V, about 1 V to about 10 V, about 1 V to about 12 V, about 1 V to about 14 V, about 1 V to about 16 V, about 1 V to about 18 V, about 1 V to about 20 V, about 2 V to about 4 V, about 2 V to about 6 V, about 2 V to about 8 V, about 2 V to about 10 V, about 2 V to about 12 V, about 2 V to about 14 V, about 2 V to about 16 V, about 2 V to about 18 V, about 2 V to about 20 V, about 4 V to about 6 V, about 4 V to about 8 V, about 4 V to about 10 V, about 4 V to about 12 V, about 4 V to about 14 V, about 4 V to about 16 V, about 4 V to about 18 V, about 4 V to about 20 V, about 6 V to about 8 V, about 6 V to about 10 V, about 6 V to about 12 V, about 6 V to about 14 V, about 6 V to about 16 V, about 6 V to about 18 V, about 6 V to about 20 V, about 8 V to about 10 V, about 8 V to about 12 V, about 8 V to about 14 V, about 8 V to about 16 V, about 8 V to about 18 V, about 8 V to about 20 V, about 10 V to about 12 V, about 10 V to about 14 V, about 10 V to about 16 V, about 10 V to about 18 V, about 10 V to about 20 V, about 12 V to about 14 V, about 12 V to about 16 V, about 12 V to about 18 V, about 12 V to about 20 V, about 14 V to about 16 V, about 14 V to about 18 V, about 14 V to about 20 V, about 16 V to about 18 V, about 16 V to about 20 V, or about 18 V to about 20 V. The controlled voltage can be about 1 V, about 2 V, about 4 V, about 6 V, about 8 V, about 10 V, about 12 V, about 14 V, about 16 V, about 18 V, or about 20 V. The controlled voltage can be at least about 1 V, about 2 V, about 4 V, about 6 V, about 8 V, about 10 V, about 12 V, about 14 V, about 16 V, or about 18 V. The controlled voltage can be at most about 2 V, about 4 V, about 6 V, about 8 V, about 10 V, about 12 V, about 14 V, about 16 V, about 18 V, or about 20 V.

The controlled voltage can be from about 1 V to about 10 V. The controlled voltage can be from about 1 V to about 2 V, about 1 V to about 3 V, about 1 V to about 4 V, about 1 V to about 5 V, about 1 V to about 6 V, about 1 V to about 7 V, about 1 V to about 8 V, about 1 V to about 9 V, about 1 V to about 10 V, about 2 V to about 3 V, about 2 V to about 4 V, about 2 V to about 5 V, about 2 V to about 6 V, about 2 V to about 7 V, about 2 V to about 8 V, about 2 V to about 9 V, about 2 V to about 10 V, about 3 V to about 4 V, about 3 V to about 5 V, about 3 V to about 6 V, about 3 V to about 7 V, about 3 V to about 8 V, about 3 V to about 9 V, about 3 V to about 10 V, about 4 V to about 5 V, about 4 V to about 6 V, about 4 V to about 7 V, about 4 V to about 8 V, about 4 V to about 9 V, about 4 V to about 10 V, about 5 V to about 6 V, about 5 V to about 7 V, about 5 V to about 8 V, about 5 V to about 9 V, about 5 V to about 10 V, about 6 V to about 7 V, about 6 V to about 8 V, about 6 V to about 9 V, about 6 V to about 10 V, about 7 V to about 8 V, about 7 V to about 9 V, about 7 V to about 10 V, about 8 V to about 9 V, about 8 V to about 10 V, or about 9 V to about 10 V. The controlled voltage can be about 1 V, about 2 V, about 3 V, about 4 V, about 5 V, about 6 V, about 7 V, about 8 V, about 9 V, or about 10 V. The controlled voltage can be at least about 1 V, about 2 V, about 3 V, about 4 V, about 5 V, about 6 V, about 7 V, about 8 V, or about 9 V. The controlled voltage can be at most about 2 V, about 3 V, about 4 V, about 5 V, about 6 V, about 7 V, about 8 V, about 9 V, or about 10 V.

The controlled voltage can be from about 4 V to about 10 V. The controlled voltage can be from about 4 V to about 5 V, about 4 V to about 6 V, about 4 V to about 7 V, about 4 V to about 8 V, about 4 V to about 9 V, about 4 V to about 10 V, about 5 V to about 6 V, about 5 V to about 7 V, about 5 V to about 8 V, about 5 V to about 9 V, about 5 V to about 10 V, about 6 V to about 7 V, about 6 V to about 8 V, about 6 V to about 9 V, about 6 V to about 10 V, about 7 V to about 8 V, about 7 V to about 9 V, about 7 V to about 10 V, about 8 V to about 9 V, about 8 V to about 10 V, or about 9 V to about 10 V. The controlled voltage can be about 4 V, about 5 V, about 6 V, about 7 V, about 8 V, about 9 V, or about 10 V. The controlled voltage can be at least about 4 V, about 5 V, about 6 V, about 7 V, about 8 V, or about 9 V. The controlled voltage can be at most about 5 V, about 6 V, about 7 V, about 8 V, about 9 V, or about 10 V.

The energy pulses can have a short duration. The duration can be from microseconds to milliseconds. The duration can be from about 0.001 ms to about 150 ms. The duration can be from about 0.001 ms to about 0.01 ms, about 0.001 ms to about 0.05 ms, about 0.001 ms to about 0.1 ms, about 0.001 ms to about 0.5 ms, about 0.001 ms to about 1 ms, about 0.001 ms to about 5 ms, about 0.001 ms to about 10 ms, about 0.001 ms to about 25 ms, about 0.001 ms to about 50 ms, about 0.001 ms to about 100 ms, about 0.001 ms to about 150 ms, about 0.01 ms to about 0.05 ms, about 0.01 ms to about 0.1 ms, about 0.01 ms to about 0.5 ms, about 0.01 ms to about 1 ms, about 0.01 ms to about 5 ms, about 0.01 ms to about 10 ms, about 0.01 ms to about 25 ms, about 0.01 ms to about 50 ms, about 0.01 ms to about 100 ms, about 0.01 ms to about 150 ms, about 0.05 ms to about 0.1 ms, about 0.05 ms to about 0.5 ms, about 0.05 ms to about 1 ms, about 0.05 ms to about 5 ms, about 0.05 ms to about 10 ms, about 0.05 ms to about 25 ms, about 0.05 ms to about 50 ms, about 0.05 ms to about 100 ms, about 0.05 ms to about 150 ms, about 0.1 ms to about 0.5 ms, about 0.1 ms to about 1 ms, about 0.1 ms to about 5 ms, about 0.1 ms to about 10 ms, about 0.1 ms to about 25 ms, about 0.1 ms to about 50 ms, about 0.1 ms to about 100 ms, about 0.1 ms to about 150 ms, about 0.5 ms to about 1 ms, about 0.5 ms to about 5 ms, about 0.5 ms to about 10 ms, about 0.5 ms to about 25 ms, about 0.5 ms to about 50 ms, about 0.5 ms to about 100 ms, about 0.5 ms to about 150 ms, about 1 ms to about 5 ms, about 1 ms to about 10 ms, about 1 ms to about 25 ms, about 1 ms to about 50 ms, about 1 ms to about 100 ms, about 1 ms to about 150 ms, about 5 ms to about 10 ms, about 5 ms to about 25 ms, about 5 ms to about 50 ms, about 5 ms to about 100 ms, about 5 ms to about 150 ms, about 10 ms to about 25 ms, about 10 ms to about 50 ms, about 10 ms to about 100 ms, about 10 ms to about 150 ms, about 25 ms to about 50 ms, about 25 ms to about 100 ms, about 25 ms to about 150 ms, about 50 ms to about 100 ms, about 50 ms to about 150 ms, or about 100 ms to about 150 ms. The duration can be about 0.001 ms, about 0.01 ms, about 0.05 ms, about 0.1 ms, about 0.5 ms, about 1 ms, about 5 ms, about 10 ms, about 25 ms, about 50 ms, about 100 ms, or about 150 ms. The duration can be at least about 0.001 ms, about 0.01 ms, about 0.05 ms, about 0.1 ms, about 0.5 ms, about 1 ms, about 5 ms, about 10 ms, about 25 ms, about 50 ms, or about 100 ms. The duration can be at most about 0.01 ms, about 0.05 ms, about 0.1 ms, about 0.5 ms, about 1 ms, about 5 ms, about 10 ms, about 25 ms, about 50 ms, about 100 ms, or about 150 ms.

The duration can be from about 20 ms to about 100 ms. The duration can be from about 20 ms to about 30 ms, about 20 ms to about 40 ms, about 20 ms to about 50 ms, about 20 ms to about 60 ms, about 20 ms to about 70 ms, about 20 ms to about 80 ms, about 20 ms to about 90 ms, about 20 ms to about 100 ms, about 30 ms to about 40 ms, about 30 ms to about 50 ms, about 30 ms to about 60 ms, about 30 ms to about 70 ms, about 30 ms to about 80 ms, about 30 ms to about 90 ms, about 30 ms to about 100 ms, about 40 ms to about 50 ms, about 40 ms to about 60 ms, about 40 ms to about 70 ms, about 40 ms to about 80 ms, about 40 ms to about 90 ms, about 40 ms to about 100 ms, about 50 ms to about 60 ms, about 50 ms to about 70 ms, about 50 ms to about 80 ms, about 50 ms to about 90 ms, about 50 ms to about 100 ms, about 60 ms to about 70 ms, about 60 ms to about 80 ms, about 60 ms to about 90 ms, about 60 ms to about 100 ms, about 70 ms to about 80 ms, about 70 ms to about 90 ms, about 70 ms to about 100 ms, about 80 ms to about 90 ms, about 80 ms to about 100 ms, or about 90 ms to about 100 ms. The duration can be about 20 ms, about 30 ms, about 40 ms, about 50 ms, about 60 ms, about 70 ms, about 80 ms, about 90 ms, or about 100 ms. The duration can be at least about 20 ms, about 30 ms, about 40 ms, about 50 ms, about 60 ms, about 70 ms, about 80 ms, or about 90 ms. The duration can be at most about 30 ms, about 40 ms, about 50 ms, about 60 ms, about 70 ms, about 80 ms, about 90 ms, or about 100 ms. Because the conductor is low mass and pulses can be brief, the energy per capsulotomy is small, making battery operation practical.

The controller can be used in stand-alone manual mode, with the surgeon activating pulses via a foot pedal or button, or in integrated mode with a robotic system, where a robotic controller sends digital commands (e.g., via USB, controlled area network (CAN), or other interface) to trigger pulses when the robotic arm has positioned the frame appropriately.

### Robotic Integration

The handpiece or an alternative adapter may be grasped or directly mounted by a robotic arm. The robotic arm can position the distal assembly in the anterior chamber, perform insertion and deployment movements, and then bring the contact frame into gentle contact with the anterior capsule.

The robotic system may use imaging or tracking to approximate the capsule location and orientation. The robotic system may advance the contact frame until a pre-set force or displacement is reached. The robotic system may rely on the gimbal to fine-tune alignment of the frame with the capsule surface.

The robotic controller can signal the pulse controller to deliver one or more DC pulses through the closed-loop conductor. The frame can complete the capsulotomy rapidly, for example in less than 0.1 second as described above, with active energy delivery.

A tool-change station can hold multiple disposable tips with different frame sizes and geometries. A robotic arm can dock with a station, release a used tip, and attach a new one according to procedure requirements.

### Delivery Devices

In some cases, the systems disclosed herein can include three primary parts: the handpiece (variations shown in FIGS. 7A-7F), the intermediate delivery device at the distal end of the handpiece, and the cutting element at the distal end of the delivery device.

The cutting elements can be delivered by a variety of delivery devices coupled to a distal end of the handpiece. The cutting elements can be delivered by a microendoscopic integration device, where the cutting element can be delivered via an endoscope channel. The cutting elements can be delivered by a robotic compatible device with a rigid interface allowing robotic cataract systems to control deployment. The cutting elements can be delivered by a dual-mode probe, which can have one channel for loop deployment and one or more channels for irrigation and/or aspiration. The delivery devices can be responsible for folding the cutting elements into tubular or elliptical configurations for insertion and/or removal from the eye.

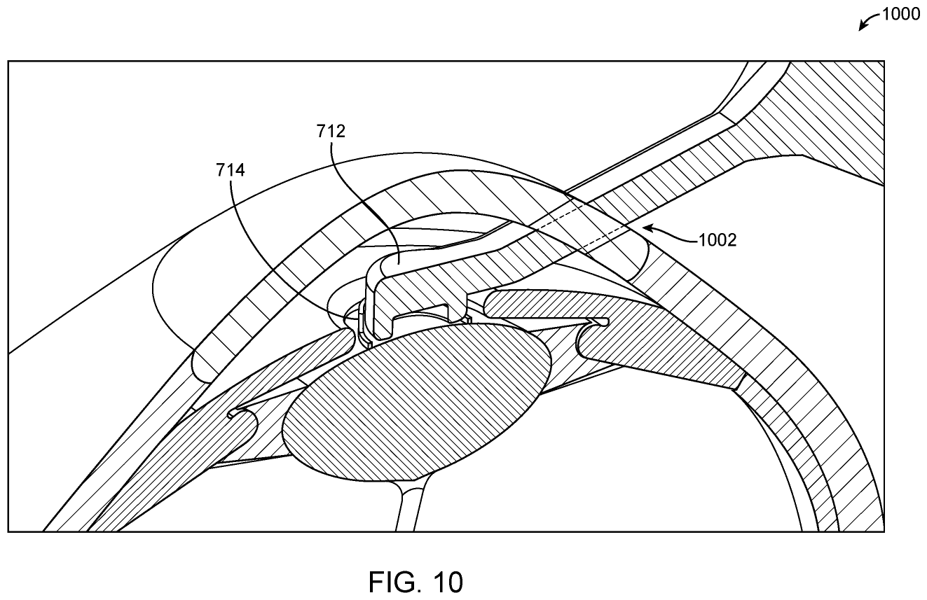

The cutting elements can be delivered by a handpiece comprising an ergonomic probe with insertion and deployment mechanisms. This can be similar to the handpiece of FIGS. 7 and 10. FIG. 10 shows cutting element delivery method 1000 comprising tip 712 and cutting element 714 in contact with the capsular bag after being inserted through corneal incision 1002.

The cutting elements can be delivered by a pen-style device and handpiece comprising a cylindrical body. This can be used for single loop deployment. The pen-style device can be similar to the device of FIGS. 1-5.

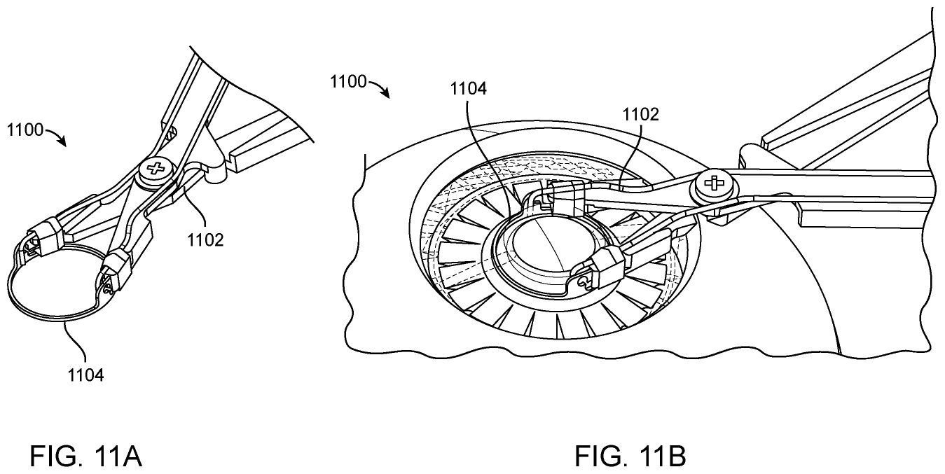

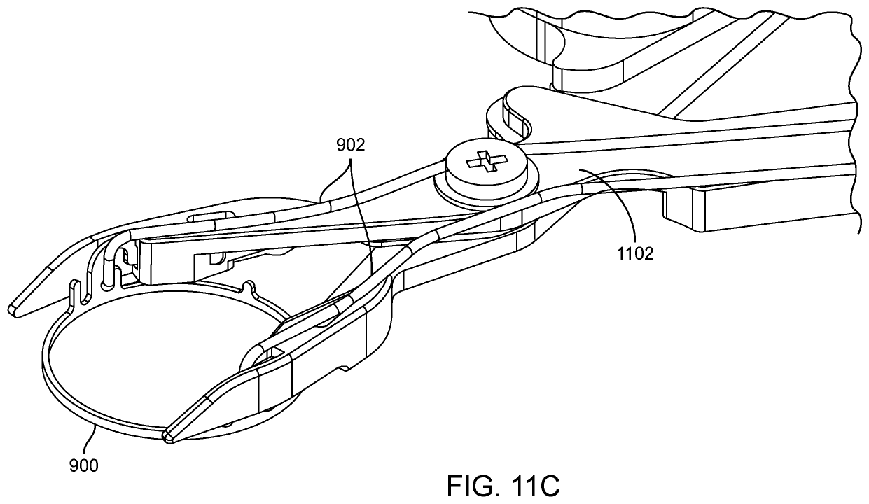

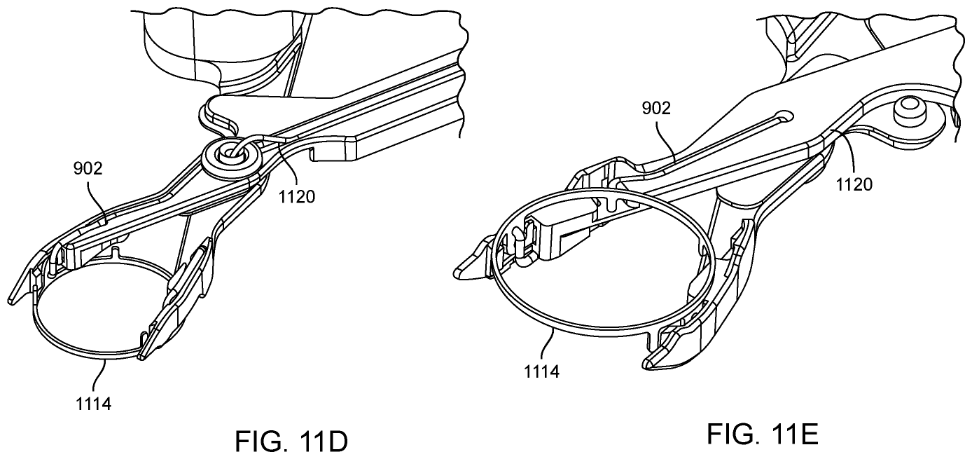

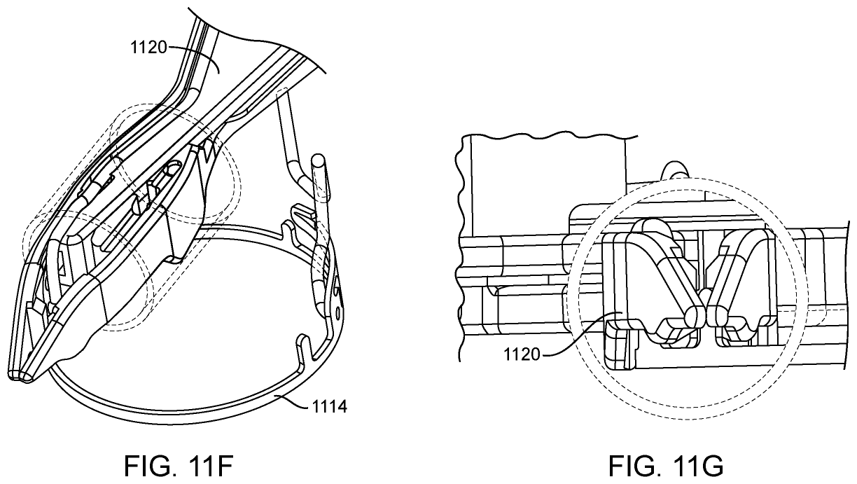

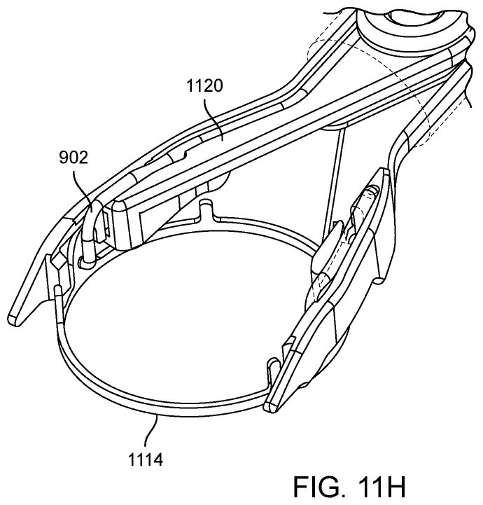

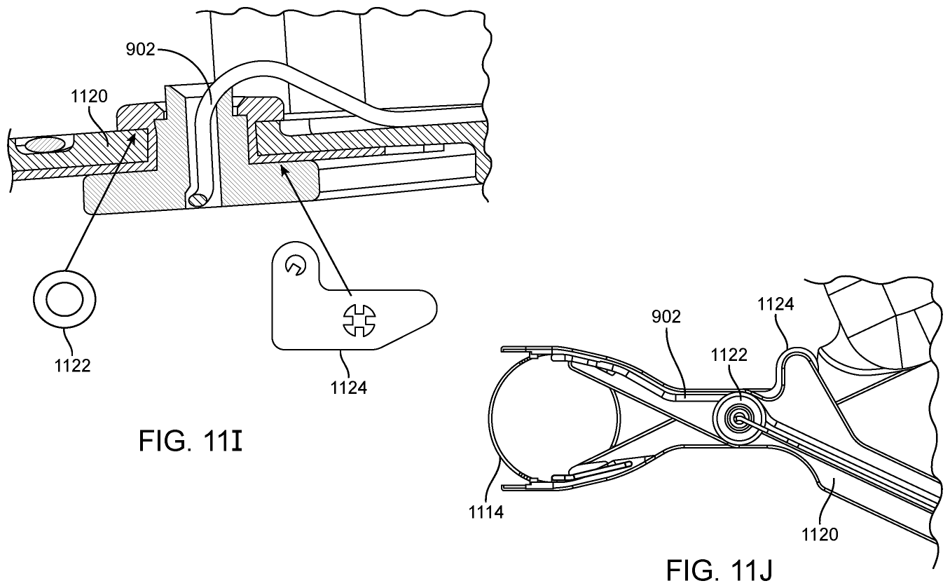

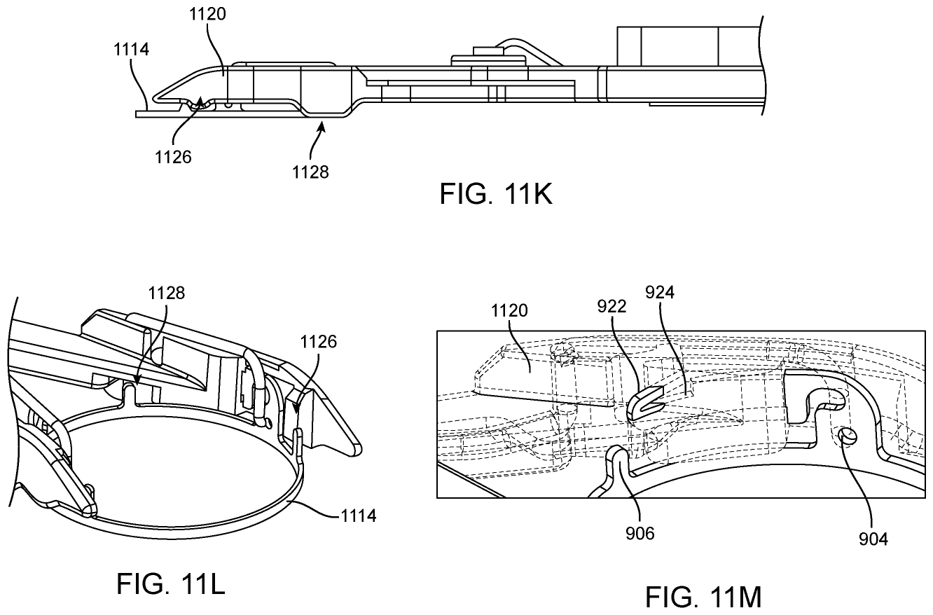

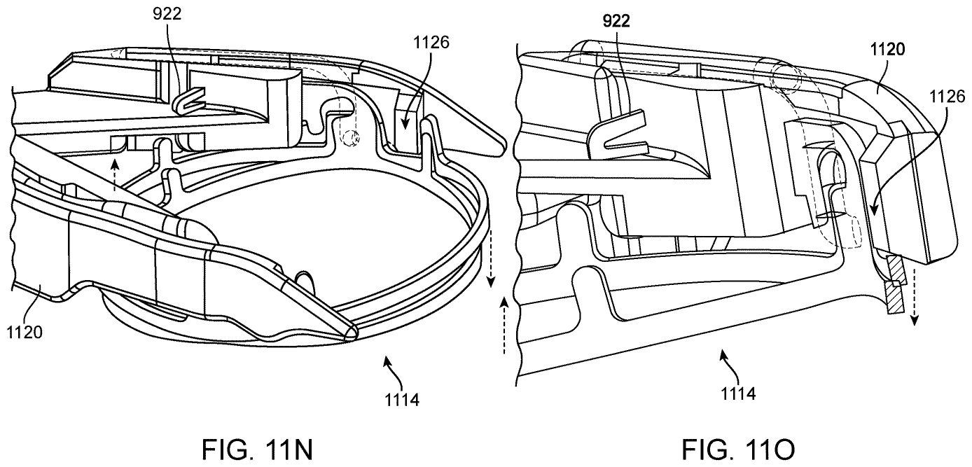

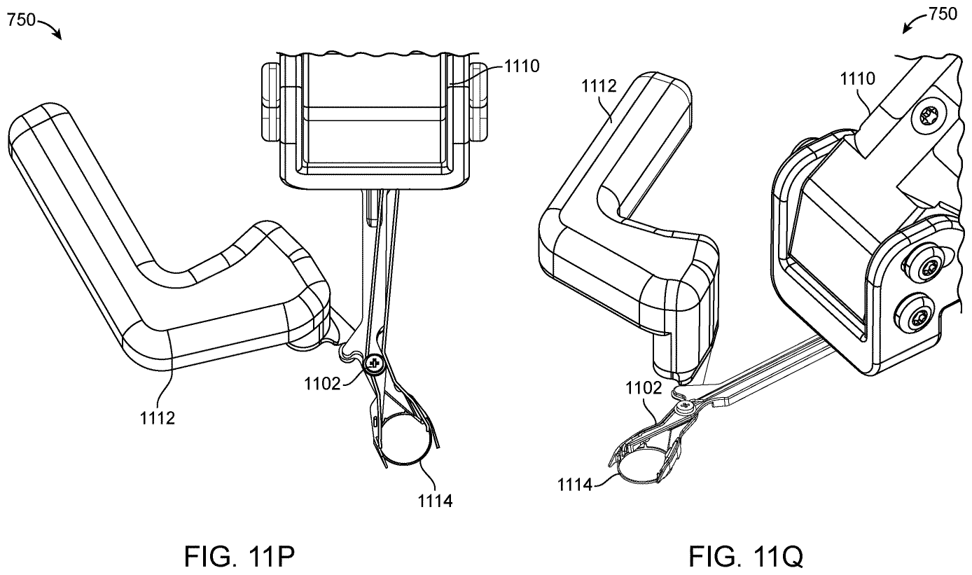

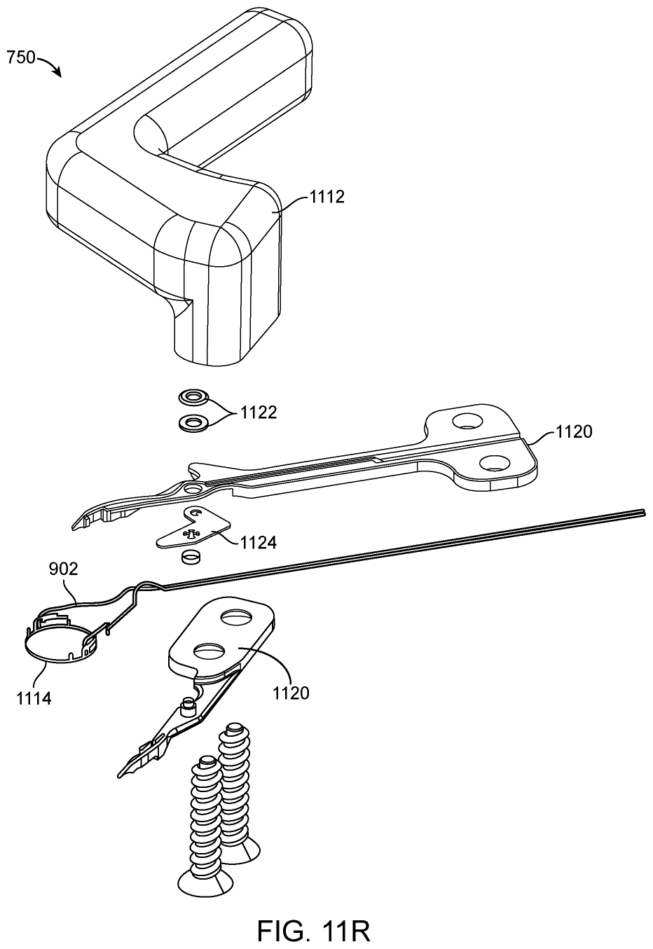

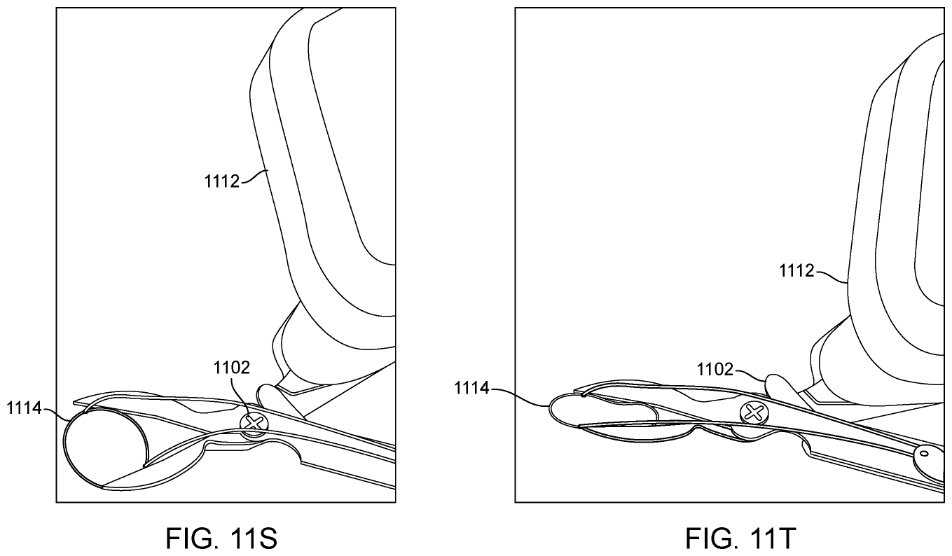

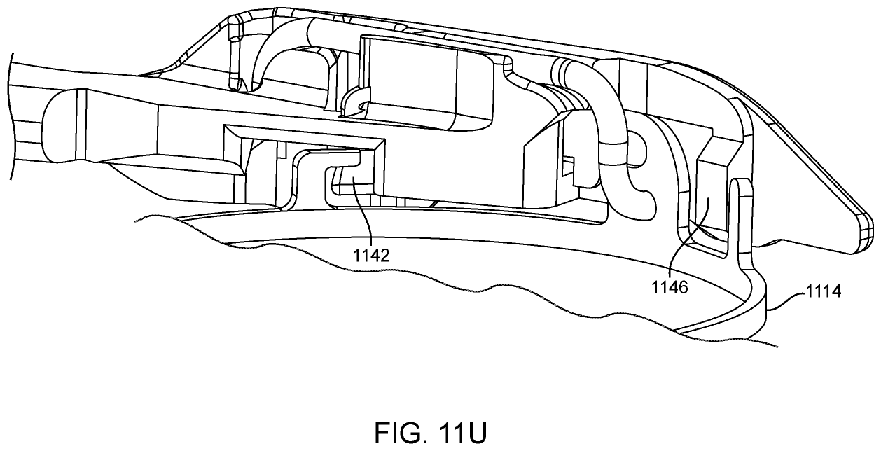

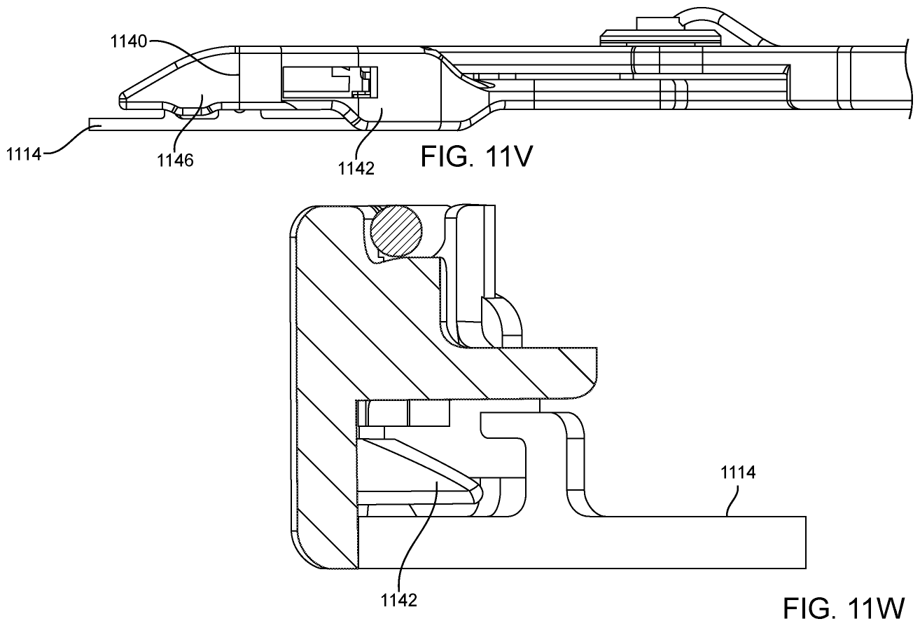

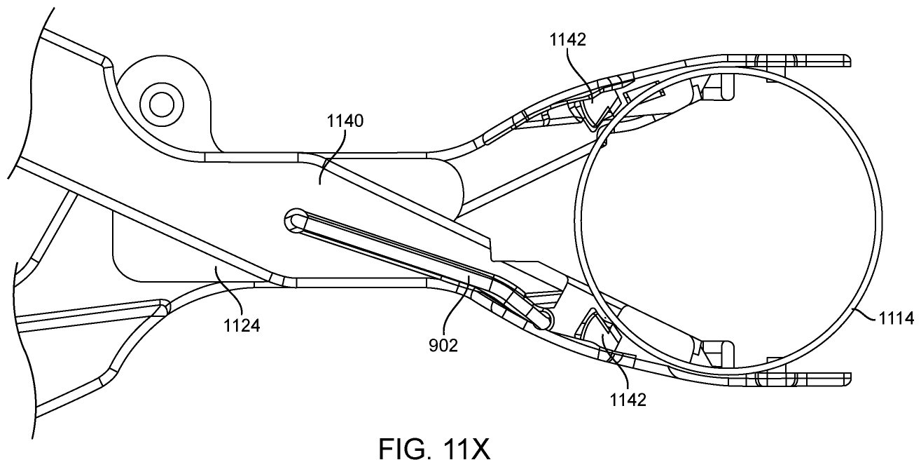

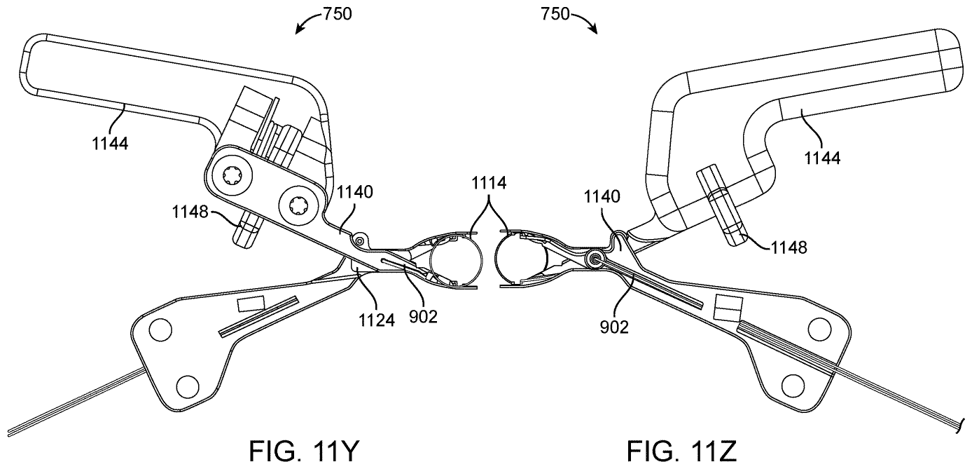

The cutting elements can be delivered by a forceps-style delivery device coupled to a handpiece. The forceps-style device can comprise twin arms deploying a loop between tips. This can also be considered a scissors-style delivery device. FIGS. 11A-11B show the forceps-style device 1102 being used to deliver cutting element 1104 in delivery method 1100. FIG. 11C shows the forceps-style/scissors device 1102 being used to deliver cutting element 900 using wires 902. In some cases, one or more wires may extend along the forceps and hook into features having holes similar to the snap features of the cutting element described above. To aid in delivery, the forceps-style device can spread the forceps to elongate the cutting element (making it more elliptical or oval) for ease of insertion into the eye prior to use of the cutting element and removal from the eye after use. The interaction of the base of the forceps may determine the amount of elongation or contraction (e.g., just as scissors may not open farther than permitted by the two bases and how they interact, the forceps may likewise be limited in opening). A limiter may also limit how much the forceps can close to retain a desired circular or elliptical shape of the cutting element. In the scissors variation, the two ends of the scissors (e.g., scissor linkages) can come together to compress or squeeze the cutting element, thereby elongating the non-squeezed parts of the cutting elements for folding and insertion as shown in FIGS. 11U-11V.

FIGS. 11D-11Q show an alternate scissors-style device 1120 for holding cutting element 1114 with closed conductor loop wires 902. Cutting element 1114 can comprise any of the above cutting elements (e.g., 900, 920, 1104, 714, etc.). Unlike the scissors device 1102, which has each of the wires on either side of a central screw, scissors device 1120 can comprise a central washer with one wire proceeding through the washer to the other side as shown in FIGS. 11D-11E. Device 1120 can allow for one or more of easier extraction, cleaner wire routing through the washer, removal of the screw to hold the pivot together, strengthened tips and easier ring attachment. Device 1120 can be optimized for a 5 mm contact frame ring, whereas device 1102 can be optimized for a 5.8 mm contact frame ring.

The position of FIGS. 11F-11G can be used to show the width of the tip of the delivery device holding the contact frame. The delivery device, when closed, can fit within a diameter. The diameter can be about 2.2 mm. The diameter can be from about 0.5 mm to about 5 mm. The diameter can be from about 0.5 mm to about 1 mm, about 0.5 mm to about 1.5 mm, about 0.5 mm to about 2 mm, about 0.5 mm to about 2.5 mm, about 0.5 mm to about 3 mm, about 0.5 mm to about 3.5 mm, about 0.5 mm to about 4 mm, about 0.5 mm to about 4.5 mm, about 0.5 mm to about 5 mm, about 1 mm to about 1.5 mm, about 1 mm to about 2 mm, about 1 mm to about 2.5 mm, about 1 mm to about 3 mm, about 1 mm to about 3.5 mm, about 1 mm to about 4 mm, about 1 mm to about 4.5 mm, about 1 mm to about 5 mm, about 1.5 mm to about 2 mm, about 1.5 mm to about 2.5 mm, about 1.5 mm to about 3 mm, about 1.5 mm to about 3.5 mm, about 1.5 mm to about 4 mm, about 1.5 mm to about 4.5 mm, about 1.5 mm to about 5 mm, about 2 mm to about 2.5 mm, about 2 mm to about 3 mm, about 2 mm to about 3.5 mm, about 2 mm to about 4 mm, about 2 mm to about 4.5 mm, about 2 mm to about 5 mm, about 2.5 mm to about 3 mm, about 2.5 mm to about 3.5 mm, about 2.5 mm to about 4 mm, about 2.5 mm to about 4.5 mm, about 2.5 mm to about 5 mm, about 3 mm to about 3.5 mm, about 3 mm to about 4 mm, about 3 mm to about 4.5 mm, about 3 mm to about 5 mm, about 3.5 mm to about 4 mm, about 3.5 mm to about 4.5 mm, about 3.5 mm to about 5 mm, about 4 mm to about 4.5 mm, about 4 mm to about 5 mm, or about 4.5 mm to about 5 mm. The diameter can be about 0.5 mm, about 1 mm, about 1.5 mm, about 2 mm, about 2.5 mm, about 3 mm, about 3.5 mm, about 4 mm, about 4.5 mm, or about 5 mm. The diameter can be at least about 0.5 mm, about 1 mm, about 1.5 mm, about 2 mm, about 2.5 mm, about 3 mm, about 3.5 mm, about 4 mm, or about 4.5 mm. The diameter can be at most about 1 mm, about 1.5 mm, about 2 mm, about 2.5 mm, about 3 mm, about 3.5 mm, about 4 mm, about 4.5 mm, or about 5 mm.

The throat of the scissor device circled in FIG. 11H, and thus the distal end of the scissor device as a whole, can fit within a diameter of the incision area during an operation or procedure. The diameter of the incision area can be about 2.4 mm. The diameter of the incision area can be from about 0.5 mm to about 5 mm. The diameter of the incision area can be from about 0.5 mm to about 1 mm, about 0.5 mm to about 1.5 mm, about 0.5 mm to about 2 mm, about 0.5 mm to about 2.5 mm, about 0.5 mm to about 3 mm, about 0.5 mm to about 3.5 mm, about 0.5 mm to about 4 mm, about 0.5 mm to about 4.5 mm, about 0.5 mm to about 5 mm, about 1 mm to about 1.5 mm, about 1 mm to about 2 mm, about 1 mm to about 2.5 mm, about 1 mm to about 3 mm, about 1 mm to about 3.5 mm, about 1 mm to about 4 mm, about 1 mm to about 4.5 mm, about 1 mm to about 5 mm, about 1.5 mm to about 2 mm, about 1.5 mm to about 2.5 mm, about 1.5 mm to about 3 mm, about 1.5 mm to about 3.5 mm, about 1.5 mm to about 4 mm, about 1.5 mm to about 4.5 mm, about 1.5 mm to about 5 mm, about 2 mm to about 2.5 mm, about 2 mm to about 3 mm, about 2 mm to about 3.5 mm, about 2 mm to about 4 mm, about 2 mm to about 4.5 mm, about 2 mm to about 5 mm, about 2.5 mm to about 3 mm, about 2.5 mm to about 3.5 mm, about 2.5 mm to about 4 mm, about 2.5 mm to about 4.5 mm, about 2.5 mm to about 5 mm, about 3 mm to about 3.5 mm, about 3 mm to about 4 mm, about 3 mm to about 4.5 mm, about 3 mm to about 5 mm, about 3.5 mm to about 4 mm, about 3.5 mm to about 4.5 mm, about 3.5 mm to about 5 mm, about 4 mm to about 4.5 mm, about 4 mm to about 5 mm, or about 4.5 mm to about 5 mm. The diameter of the incision area can be about 0.5 mm, about 1 mm, about 1.5 mm, about 2 mm, about 2.5 mm, about 3 mm, about 3.5 mm, about 4 mm, about 4.5 mm, or about 5 mm. The diameter of the incision area can be at least about 0.5 mm, about 1 mm, about 1.5 mm, about 2 mm, about 2.5 mm, about 3 mm, about 3.5 mm, about 4 mm, or about 4.5 mm. The diameter of the incision area can be at most about 1 mm, about 1.5 mm, about 2 mm, about 2.5 mm, about 3 mm, about 3.5 mm, about 4 mm, about 4.5 mm, or about 5 mm.

FIG. 11I shows an inner view of scissor device 1120 with wire 902 extending through the device. The wire route can be altered after swagging. Device 1120 can comprise a round washer 1122 and a second washer 1124. The first washer, or round washer 1122, can be disposed externally to the scissor device, as is shown in FIG. 11J, and the device 1120 can have a built-in rivet alongside the washer. The second washer 1124 can have a shape to accommodate the shape of the scissors, both tucking between the two halves of the scissors as shown in FIG. 11I and outline the halves of the scissors as shown in FIG. 11J. The washers can also be used to insulate the two halves of the scissors in device 1120. The washers can comprise polytetrafluoroethylene (PTFE).

FIGS. 11K-11L show control features for controlling the angle and motion of the contact ring 114 via proximal control feature 1128 and distal control feature 1126. These control features can also act as stabilization features. FIG. 11L shows how the proximal control feature 1128 surrounds the post 906 and how the distal control feature 1126 is located between post 906 and slot interface 908. FIG. 11M shows the placement of the ring in the scissor 1120, including hooked segment 922, flex bridge 924, post 906, and pivot pin 904. FIGS. 11N-11O show how the gimbal re-alignment cam (e.g., the distal control or stabilization feature) 1126 can drive the front (distal end) of the ring 1114 down and the back (proximal end) up when compressed by the scissors 1120. The upward and downward movement when compressed is shown with dotted arrows. The hooked segment 922 is shown where it exits from the scissors.

In some cases, handheld and finger-pressure-actuated scissor closures may be provided. FIGS. 11P-11Q show a handheld or palm-held section 1110 and a finger-actuated closure 1112 of a scissor or forceps device 1102 with a cutting element 1114. FIG. 11R shows a similar exploded view, showing the finger-actuated closure 1112 of a scissor device 1120 with washers 1122 and 1124, the cutting element 1114, and the closed-loop conductor 902. FIGS. 11P-11Q show a distal end 750 of a heating system as shown in FIGS. 7D-7E. In some cases, the section 1110 may be held/pressed by a thumb of a user while the closure 1112 is closed via one or more fingers. In some cases, one or two hands can be used. The scissor device 1102 can also comprise the scissor device 1120 as described above. The cutting element 1114 can comprise any of the above cutting elements (e.g., 900, 920, 1104, 714, etc.). As shown in FIGS. 11S-11T, a user can actuate the device (e.g., close the scissors) by decreasing the angle between the closure 1112 and the palm section 1110. A first (open) angle can be θ₁. A second (closed) angle can be θ₂. With the second angle (e.g., with θ₂), the cutting element 1114 can be pushed/squeezed/folded by the scissors into an elliptical or tubular shape for insertion into the eye.

In some cases, a cam feature may tuck in the rear portion of the cutting element when the scissor is closed. FIG. 11U shows the proximal cam feature 1142 relative to cutting element 1114 and distal control feature 1146. Distal control feature 1146 can be similar to distal control feature 1126 described above. In some cases, the proximal (e.g., rear) portion of the cutting element may catch on the bottom edge of the incision made by the cutting element. Such catching can make removing the tip from the eye difficult. Rotating the tip either about 90 degrees or about 180 degrees may improve extraction, but may not fully solve the difficulty. The proximal cam feature 1142 can tuck the cutting element in to allow for smooth retrieval without catching on edges. In testing, the proximal cam feature made extraction consistent across distal tips (e.g., scissors and cutting elements) without catching the cutting element during retrieval.

FIG. 11V shows an external side view of the same features on scissor device 1140. Scissor device 1140 can be similar to the scissor devices described above. The external side view of scissor device 1140 can be similar to the view shown in FIG. 11K of scissor device 1120. FIG. 11W shows a magnified side view of the proximal cam feature 1142 relative to the cutting element 1114. FIG. 11X shows how the proximal cam features 1142 on either side of the cutting element 1114 sit relative to the distal end of the scissor 1140 and the cutting element 1114. As with other scissor designs, the scissor 1140 can have a wire 902 comprising a closed-loop conductor running through it. The scissor 1140 can have a washer 1124 between the two halves of the scissor.

FIGS. 11Y-11Z show right and left views of scissor 1140 with handpiece 1144. Together, the scissor 1140 with handpiece 1144 can form another version of distal end 750. As described above, the scissor 1140 can comprise wire 902 comprising a closed-loop conductor and washer 1124 between the two halves of the scissor, and may hold the cutting element 1114. Just as the scissor 1140 is similar to scissor 1120, the handpiece 1144 can be similar to handpiece 1112. In some cases, handpiece 1144 may differ from handpiece 1112 by the addition of locking element 1148. Locking element 1148 allows a user to press on the button and lock the degree of opening or closure of the scissor 1140 so the device may hold the scissor shut without active user pressure (e.g., without the user exerting constant pressure to hold the scissor shut to a certain degree). In some cases, locking the scissor 1140 can be easier to lock during insertion or removal. When in situ, the lock can be pressed again to be unlocked, thereby allowing the cutting element/ablation zone to expand. The expansion and contraction can look similar to the expansion and contraction of FIGS. 11S-11T.

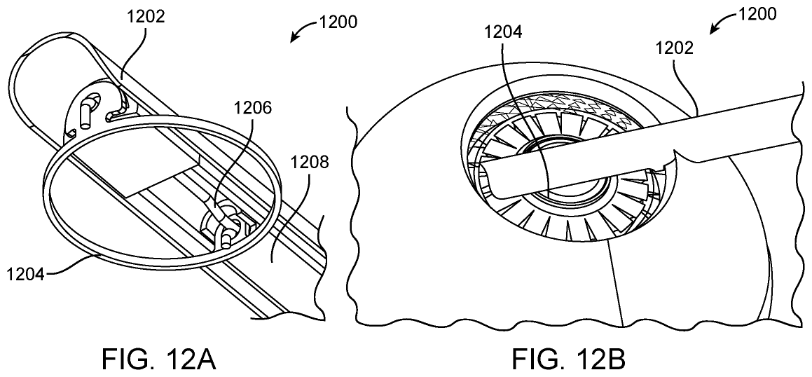

The cutting elements can be delivered by a half-tube or half-pipe device as shown in FIGS. 12A-12B. FIGS. 12A-12B show a half-tube device 1200 comprising a tube cover 1202, a cutting element 1204, an expansion limiter 1206, and shrink tubing 1208. Rather than a fully enclosed tube, the half-tube device 1200 can be open on the bottom half to carry the cutting element. In some cases, the entirety, or substantially the entirety, of the half-tube device 1200 comprises a half-tube. In some cases, only the distal tip comprises a half-tube, and the remainder of the device may comprise a full tube or a housing to store the cutting element when not in use. In some cases, a wire, such as the expansion limiter 1206, may hook into features having holes similar to the snap features of the cutting element described above. To aid in delivery, the half-tube device can spread the shrink tubing 1208 to elongate and fold the cutting element (making it more elliptical, tubular, or oval) for ease of insertion into the eye prior to use of the cutting element and removal from the eye after use. Once elongated, the cutting element may be tucked into the half-tube to minimize the overall size of the device being inserted into or removed from the eye.

The amount of elongation of the cutting element can be limited by the expansion limiter 1206. As the shrink tubing 1208 stretches the cutting element, the expansion limiter can also be stretched to its full length because it may be hooked into the proximal and distal ends of the cutting element. During use of the element, the shrink tubing 1208 can bring the sides of the cutting element together to create a more circular form. In some cases, the expansion limiter 1206 can have a stretchable component and a hard component, such that the expansion limiter 1206 can be stretched when the cutting element is elongated and the hard component can limit how much the cutting element can be brought together (e.g., the hard elements meet and may not be further pushed together). In this way, the diameter of the cutting element can be preset by the expansion limiter 1206. The expansion limiter 1206 can also be used to hold the cutting element in place throughout the delivery, heating/cutting, and/or withdrawal.

### One-Click Locking Mechanism

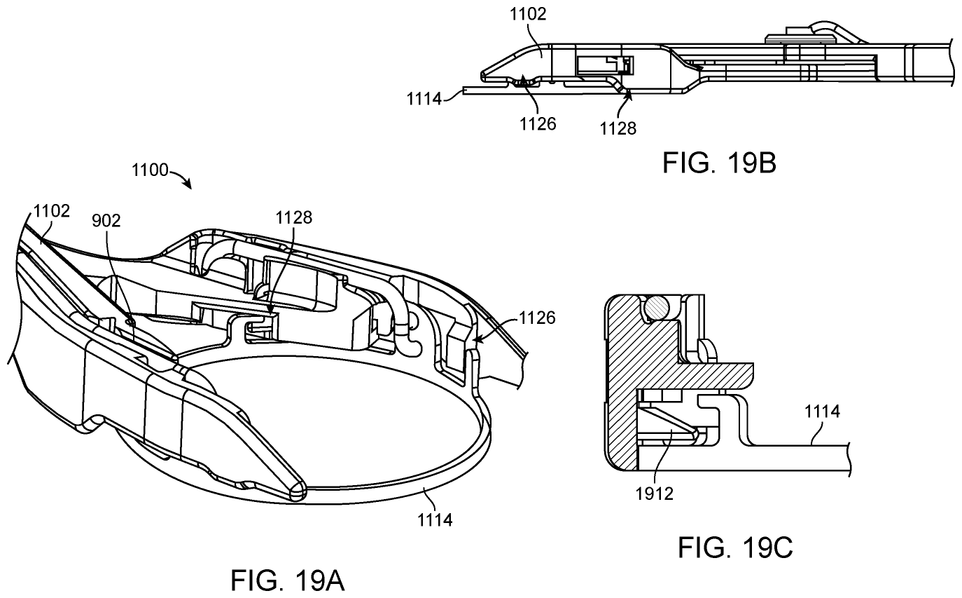

Referring generally to FIGS. 19A-24B, ring adjustment devices and delivery mechanisms for use with a thermal capsulotomy device are described according to various example embodiments. The ring adjustment devices described herein can include a one-click locking mechanism, a push button delivery device, and/or a scroll wheel delivery device, one or more of which may be configured to actuate a cutting element between an open configuration and a closed configuration. The disclosed delivery mechanisms can facilitate discrete, repeatable, and/or reproducible deployment of the cutting element while reducing reliance on continuous hand pressure by a surgeon. In operation, the delivery mechanisms described herein can allow the surgeon to transition the cutting element between a compact insertion configuration suitable for passage through a corneal incision and a deployed capsulotomy configuration in which the cutting element assumes its shape-memory geometry for contact with the capsular bag, with the transition being accomplished through mechanical actuation. Audible and/or tactile feedback may confirm discrete end-states of the delivery mechanisms, providing the surgeon with confirmation that the cutting element 1114 has reached a defined configuration before proceeding with insertion, deployment, energy delivery, or withdrawal.

Referring to FIGS. 19A-19C, example ring adjustment devices for use with the thermal device are shown. FIG. 19A illustrates a perspective view of the delivery device 1100 comprising a forceps-style device 1102, a cutting element 1114, one or more wires 902, a proximal control feature 1128, and a distal control feature 1126. FIG. 19B illustrates a side view of the forceps-style device 1102 with the cutting element 1114 and the proximal control feature 1128 and the distal control feature 1126 visible in profile. FIG. 19C illustrates a side view showing the cutting element 1114 and a proximal cam feature 1142. The forceps-style device 1102 can be configured to actuate the cutting element 1114 between contracted and expanded configurations through proximal manipulation of the proximal control feature 1128 or the distal control feature 1126.

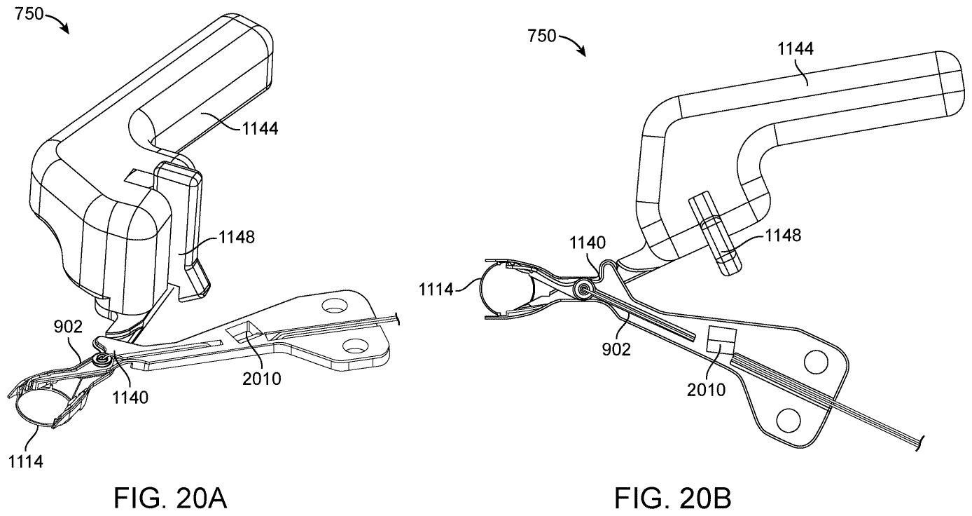

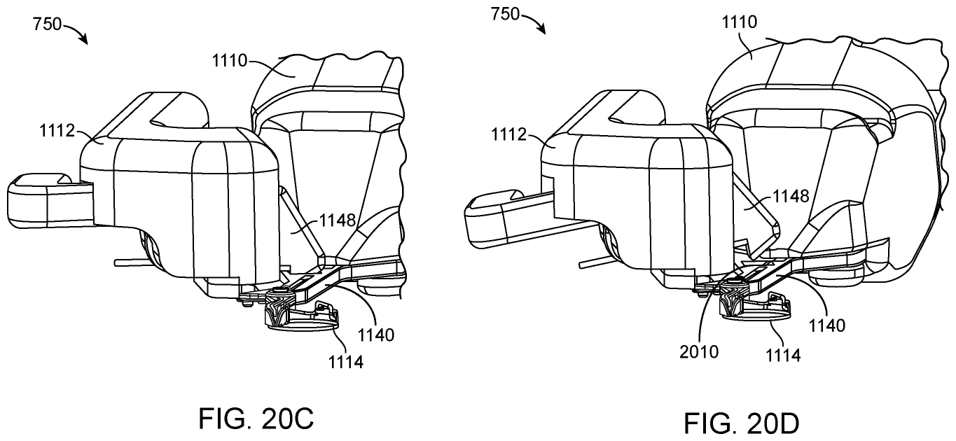

FIG. 20A shows a perspective view of an example ring adjustment device comprising the distal end 750, the cutting element 1114, the scissor 1140 (or any of the other scissor devices described herein) coupled to a handpiece 1144, one or more wires 902 extending between the handpiece 1144 and the distal end 750, a locking element 1148, and a locking element opening 2010 configured to receive the locking element 1148. FIG. 20B illustrates a top-down view of the ring adjustment device showing the distal end 750, the wire 902, the scissor 1140, the handpiece 1144, the locking element 1148, the locking element opening 2010, and the cutting element 1114. The scissor 1140 can be operatively coupled to a handheld or palm-held section 1110 and a finger-actuated closure 1112 to control movement of the cutting element 1114 between the open and closed configurations. In the open configuration, the scissor 1140 may permit the cutting element 1114 to expand to its deployed shape-memory geometry for capsulotomy. In the closed configuration, the scissor 1140 may compress the cutting element 1114 into a compact profile suitable for insertion through a corneal incision or withdrawal from the eye.

FIG. 20C shows a perspective view of the ring adjustment device with the locking mechanism engaged, showing the distal end 750, the scissor 1140, the locking element 1148, the cutting element 1114, the handheld or palm-held section 1110, and the finger-actuated closure 1112. FIG. 20D shows a perspective view of the ring adjustment device with the locking mechanism disengaged, showing the distal end 750, the scissor 1140, the locking element 1148, the locking element opening 2010, the cutting element 1114, the handheld or palm-held section 1110, and the finger-actuated closure 1112. In some embodiments, the locking element 1148 can comprise an arm, a latch, or a detent extending from the handheld or palm-held section 1110 of the handpiece 1144. When a user actuates the locking element 1148, the locking element 1148 may engage the locking element opening 2010 on an opposing portion of the device to retain the finger-actuated closure 1112 against the handheld or palm-held section 1110 and thereby maintain the scissor 1140 in the closed configuration. When the locking element 1148 is disengaged from the locking element opening 2010, the finger-actuated closure 1112 may be free to move relative to the handheld or palm-held section 1110, permitting the scissor 1140 to transition between the open and closed configurations in response to user input.

The one-click locking mechanism can allow a user to actuate the delivery device between discrete fully open and fully closed end-states without continuous hand pressure. The one-click locking mechanism can comprise the locking element 1148 configured to engage the corresponding locking element opening 2010 to retain the scissor 1140 in a selected configuration. The locking element 1148 may comprise the arm, a spring-loaded latch, or a snap-fit detent that is biased toward engagement with the locking element opening 2010 when the finger-actuated closure 1112 is pressed against the handheld or palm-held section 1110. Once the locking element 1148 is engaged, the scissor 1140 can be held in the closed or open configuration without the user exerting active pressure on the finger-actuated closure 1112 or the handheld or palm-held section 1110, thereby freeing the user's hand from sustained force during insertion, positioning, or withdrawal of the delivery device. The one-click locking mechanism can allow the device to be held in a defined configuration independently of user-applied force, thereby reducing variability during use. Engagement and disengagement of the locking element 1148 can be accompanied by audible and/or tactile feedback (e.g., an audible “click” and/or a tactile detent) that provides confirmation to the user that the device has reached a discrete locked or unlocked end-state. The audible and/or tactile feedback may allow the user to confirm the locking state without visual inspection of the locking element 1148, which can be advantageous during intraocular procedures where the user's visual attention may be directed toward the surgical field.

By mechanically defining the open and closed extents of the scissor 1140, the one-click locking mechanism can reduce reliance on hand pressure alone to define the open extent of the cutting element 1114. The mechanical definition of discrete end-states can improve reproducibility of the cutting element 1114 geometry between procedures and across users, and can enhance control during positioning and deployment of the cutting element 1114. The one-click locking mechanism can also reduce the likelihood of unintended outward expansion of the cutting element 1114 during insertion through a corneal incision, when external forces on the distal end 750 can be greatest. By maintaining the scissor 1140 in a fully closed configuration during insertion and withdrawal, the locking mechanism can maintain a compact insertion profile and can reduce the risk of the cutting element 1114 catching on wound tissue during withdrawal. The one-click locking mechanism may thereby coordinate with the delivery device to ensure that the cutting element 1114 remains in a controlled configuration throughout the capsulotomy procedure, from insertion through energy delivery and device withdrawal.

In some embodiments, when the scissor 1140 is in the locked closed configuration, a gimballed contact frame coupled to the distal end 750 can be retracted or “tucked” posteriorly relative to the distal end 750, such that an exposed proximal edge of the frame may be reduced during withdrawal. The tuck-in behavior can be coordinated with the locking state of the device, such that locking the scissor 1140 closed can simultaneously seat the cutting element 1114 in a low-profile, snag-resistant configuration suitable for entry into and/or exit from the eye. The tuck-in behavior can complement the proximal cam feature 1142 described above, such that the proximal cam feature 1142 mechanically deflects a proximal edge of the cutting element 1114 inward when the scissor 1140 is closed, and the locked state of the scissor 1140 may maintain this deflected position throughout withdrawal. Together, the tuck-in behavior and the proximal cam feature 1142 can reduce snagging on the corneal incision during withdrawal. The gimballed contact frame can exhibit anisotropic elastic deformation, such that the frame may be shorter in one direction and can expand in a perpendicular direction when compressed, similar to the behavior of an elastic band compressed along one axis. The anisotropic deformation may allow the cutting element 1114 to assume a compact elongated profile when the scissor 1140 is closed while permitting the cutting element 1114 to return to its deployed shape-memory geometry when the scissor 1140 is opened. The gimballed contact frame can be removable and replaceable relative to the distal end 750, and can also be manipulated (e.g., pushed or pulled) without removal from the arms of the scissor 1140 or the forceps-style device 1102. In some embodiments, a retention mechanism between the gimballed contact frame and the distal end 750 can resist significant force and/or pressure during intraocular manipulation without the frame detaching from the arms, which may differentiate the retention from a fragile snap-fit coupling.

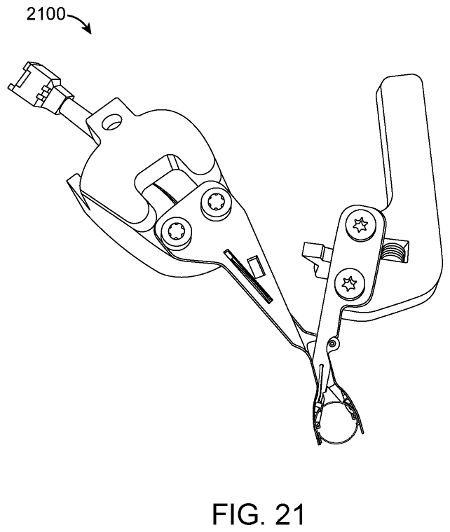

Referring to FIG. 21, a perspective view of a commercial embodiment 2100 of the delivery device is shown. The commercial embodiment 2100 represents an assembled configuration of the delivery device as manufactured for clinical use, incorporating the scissor device 1102, the cutting element 1114, and the locking mechanism described above into a single integrated unit. The commercial embodiment 2100 can be provided as a sterile, disposable assembly or as a reusable device with replaceable distal tips.

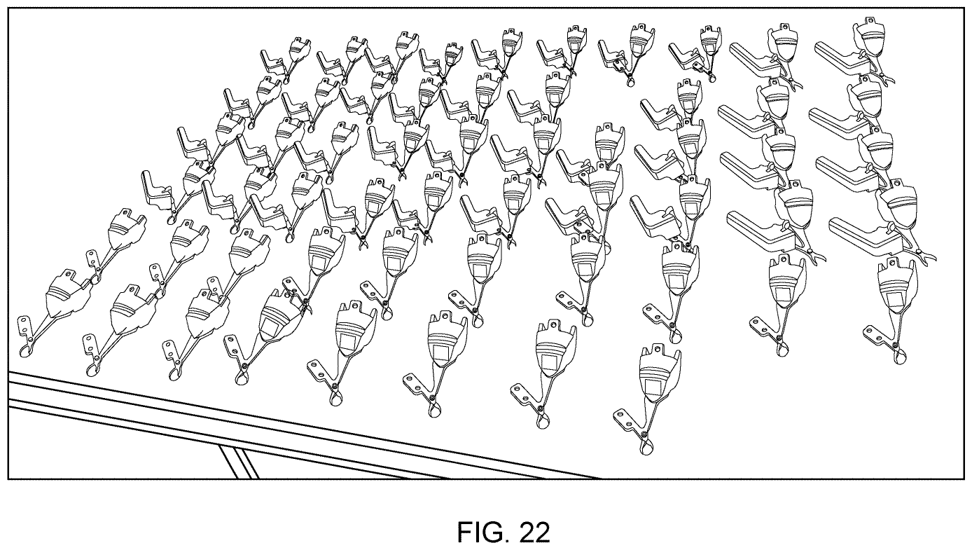

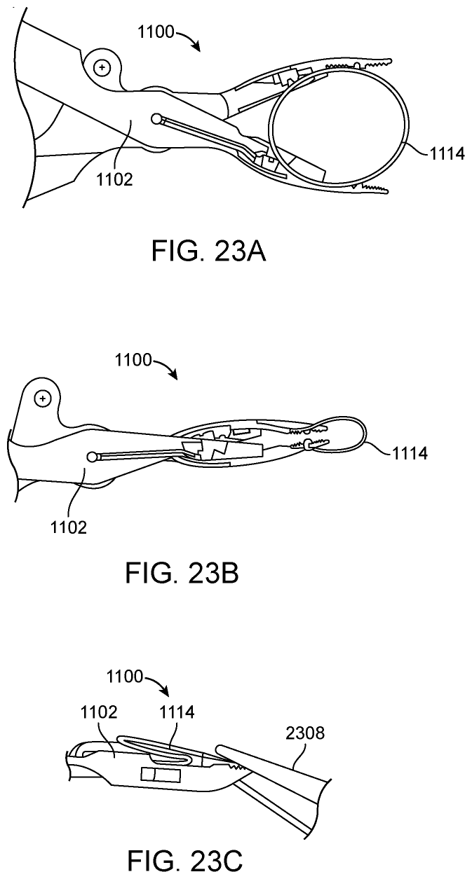

Referring to FIG. 22, example ring adjustment devices for use with the thermal device are shown. Referring to FIGS. 23A-23C, an example thermal device is shown in open and closed states. FIG. 23A illustrates a top-down view of the delivery device 1100 in an open state, comprising the forceps-style device 1102 and the cutting element 1114 in a deployed configuration. FIG. 23B illustrates a top-down view of the delivery device in a closed state, with the forceps-style device 1102 retaining the cutting element 1114 in a compact configuration. FIG. 23C illustrates a perspective view of the delivery device 1100 showing the forceps-style device 1102, the cutting element 1114, and an outside force 2308 applied to the device during use. The open and closed states shown in FIGS. 23A-23C illustrate the range of motion of the cutting element 1114 as controlled by the forceps-style device 1102. In the open state, the cutting element 1114 may assume its shape-memory geometry for contact with the capsular bag, and in the closed state, the cutting element 1114 may be compressed into a compact profile for passage through the corneal incision.

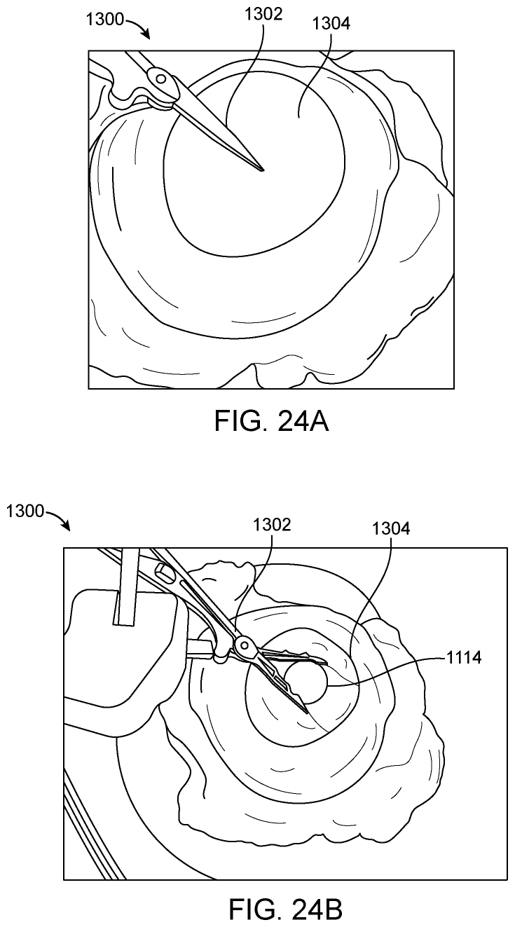

Referring to FIGS. 24A-24B, an example method for forming an opening in a capsular bag 1304 using the thermal device is shown. FIG. 24A illustrates a capsulorhexis method 1300 with a device 1302 positioned partially within the capsular bag 1304, with the cutting element in a closed state for insertion through a corneal incision to form the capsulotomy opening. FIG. 24B illustrates the capsulorhexis method 1300 with the device 1302 in an open state and the cutting element 1114 (ring) deployed within the capsular bag. The method shown in FIGS. 24A-24B can be performed using any of the delivery devices described herein, including the one-click locking mechanism, the push button delivery device, and the scroll wheel delivery device.

### Push Button Delivery Device

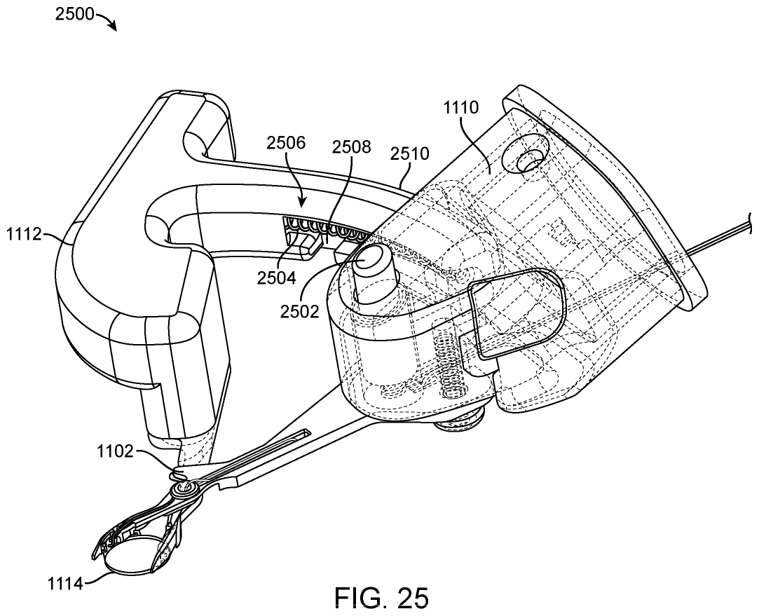

Referring to FIG. 25, a perspective view of a push button delivery device 2500 is shown. The push button delivery device 2500 can provide discrete, repeatable actuation of the cutting element 1114 between the open and closed configurations using a spring-biased, push-button-actuated release mechanism. As shown in FIG. 25, the push button delivery device 2500 comprises a scissor device 1102, a handheld or palm-held section 1110, a finger-actuated device 1112, a cutting element 1114 carried at a distal end of the scissor device 1102, a push button 2502, a compression spring 2504, an arced slot 2506, a latch opening 2508, and an arm 2510 of the finger-actuated device 1112. The push button delivery device 2500 can provide discrete end-state control similar to the one-click locking mechanism described above, while substituting spring-driven actuation for manual finger pressure as the force that drives the scissor device 1102 between its end-states. The compression spring 2504 can store mechanical energy when the arm 2510 is retained in the closed position and can release the stored energy upon actuation of the push button 2502 to drive the scissor device 1102 toward the open configuration.

The cutting element 1114 can be disposed at the distal end of the scissor device 1102 and can be transitioned between contracted and expanded configurations in response to actuation of the finger-actuated device 1112 and the push button 2502. The compression spring 2504 can be disposed along the arced slot 2506 and can bias the arm 2510 of the finger-actuated device 1112 toward an open configuration. In some embodiments, the arm 2510 can be retained, against the bias of the compression spring 2504, in a closed position by engagement with the latch opening 2508. In the closed position, the compression spring 2504 can be in a compressed state, storing potential energy along the arced slot 2506. The push button 2502 can act as a release mechanism such that actuation of the push button 2502 disengages the arm 2510 from the latch opening 2508, allowing the compression spring 2504 to drive movement of the finger-actuated device 1112. The movement of the finger-actuated device 1112 can cause the scissor device 1102 to open and thereby expand the cutting element 1114 toward a deployed configuration in which the cutting element 1114 may assume its shape-memory geometry for contact with the capsular bag. To return the push button delivery device 2500 to the closed configuration, the user may press the finger-actuated device 1112 toward the handheld or palm-held section 1110, compressing the compression spring 2504 along the arced slot 2506 until the arm 2510 re-engages the latch opening 2508. The push button 2502 can be located on a disposable tip side of the push button delivery device 2500 or on the handheld or palm-held section 1110, and can be actuated by the surgeon to release the arm 2510 from the latch opening 2508 or allow the arm 2510 to move into the closed position by allowing movement of the arm 2510 for the push button 2502 to engage the latch opening 2508.

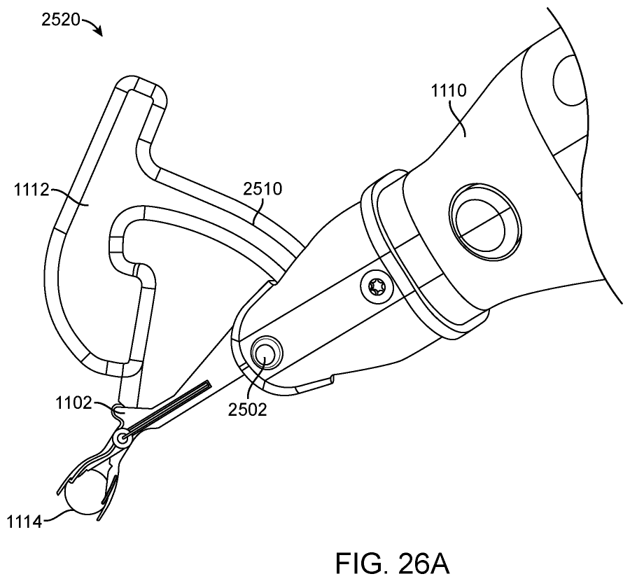

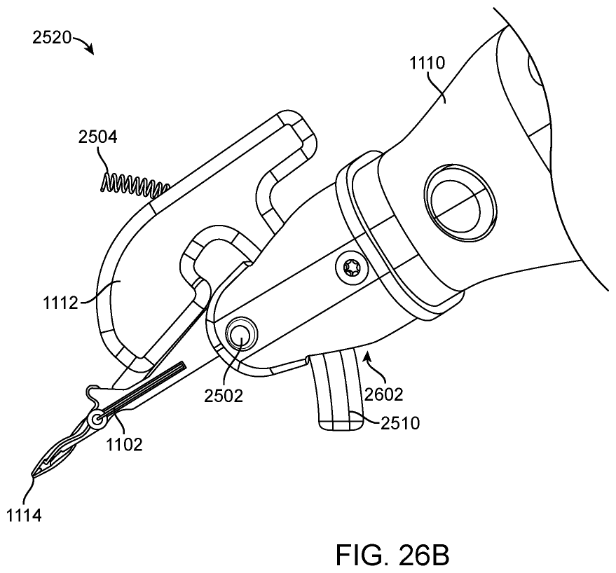

Referring to FIGS. 26A-26B, top-down views of a push button delivery device on a handle 2520 in open and closed positions are shown. FIG. 26A shows the push button delivery device on the handle 2520 in the open position, in which the arm 2510 is displaced from the latch opening 2508 by the compression spring 2504 and the cutting element 1114 is in an expanded geometry. FIG. 26B shows the push button delivery device on the handle 2520 in the closed position, in which the arm 2510 is retained at the latch opening 2508 and the compression spring 2504 is compressed along the arced slot 2506. An opening 2602 for the distal end of the finger-actuated device 1112 can be provided to accommodate the travel of the arm 2510 between the open and closed positions.

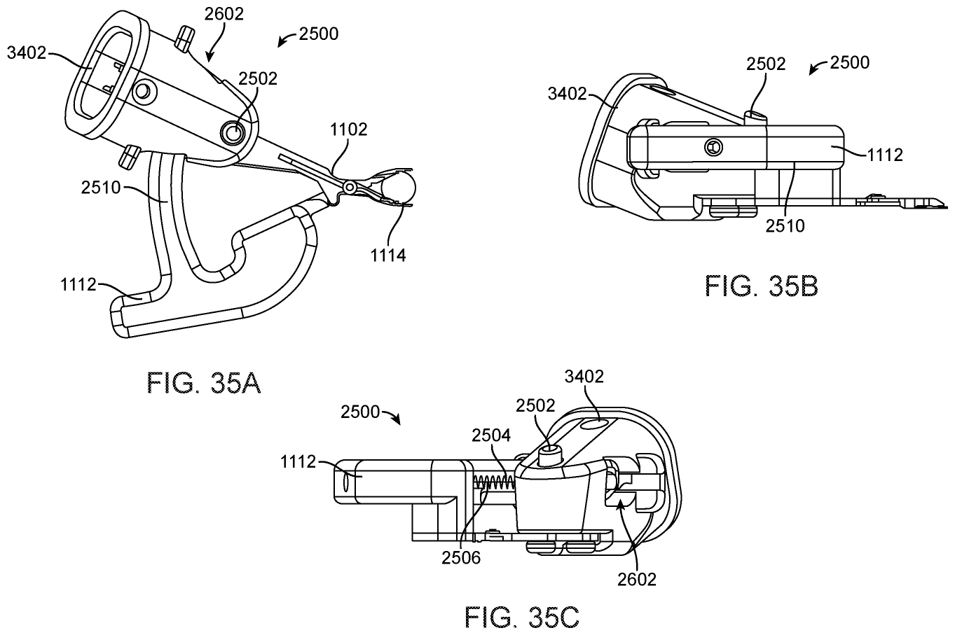

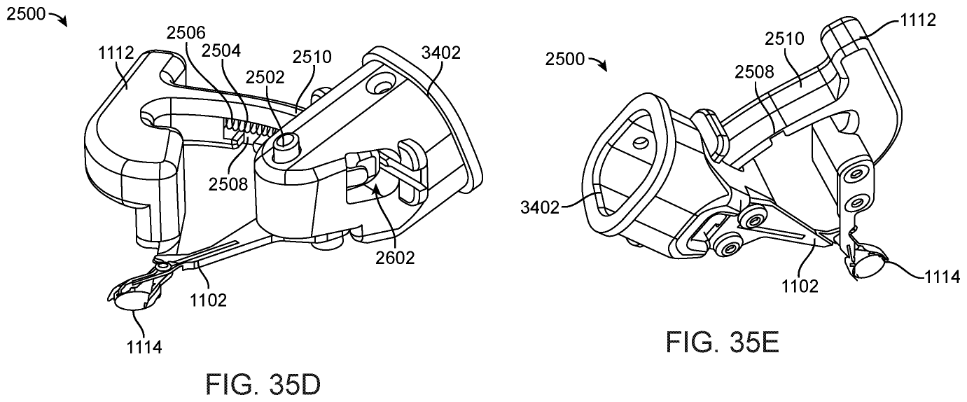

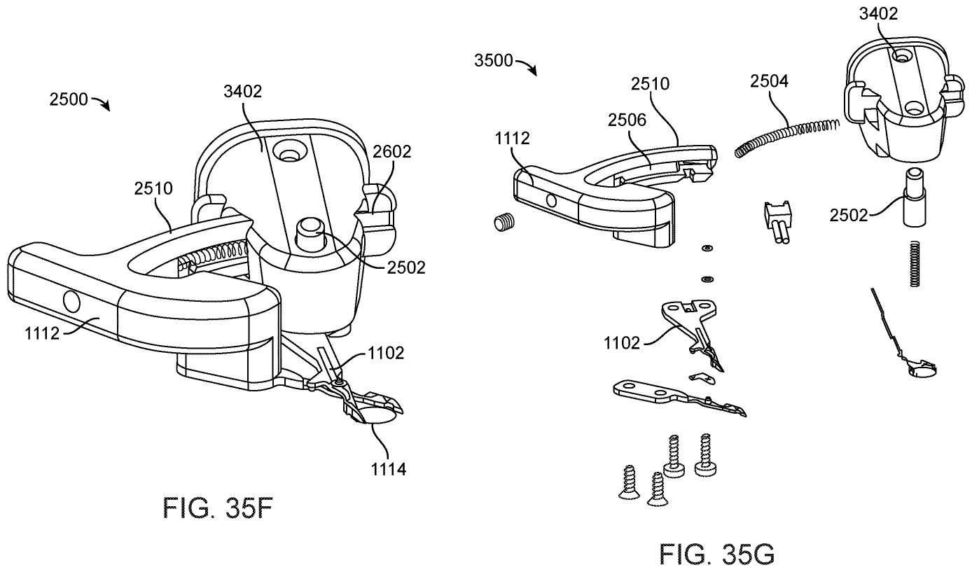

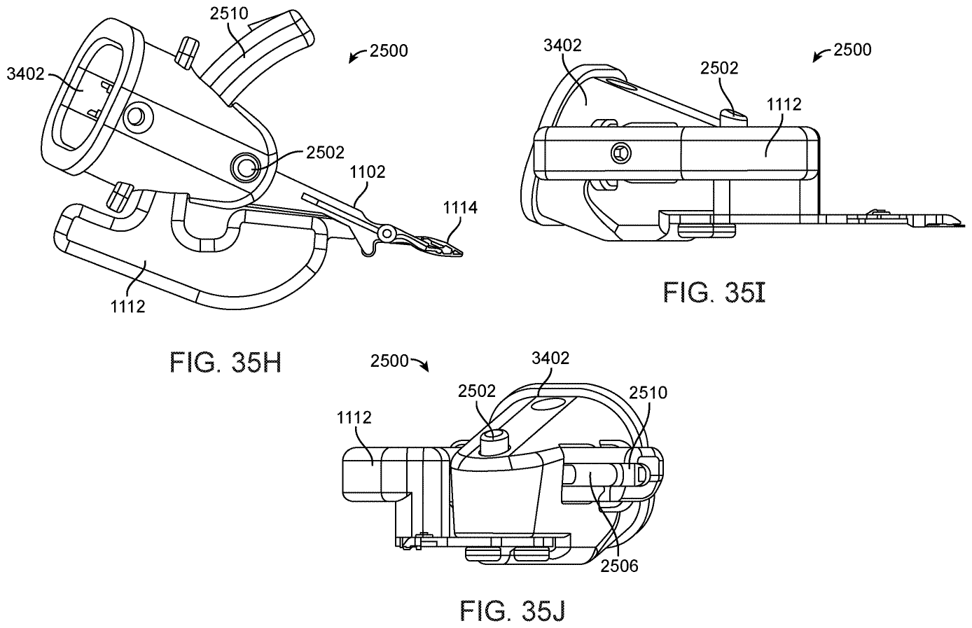

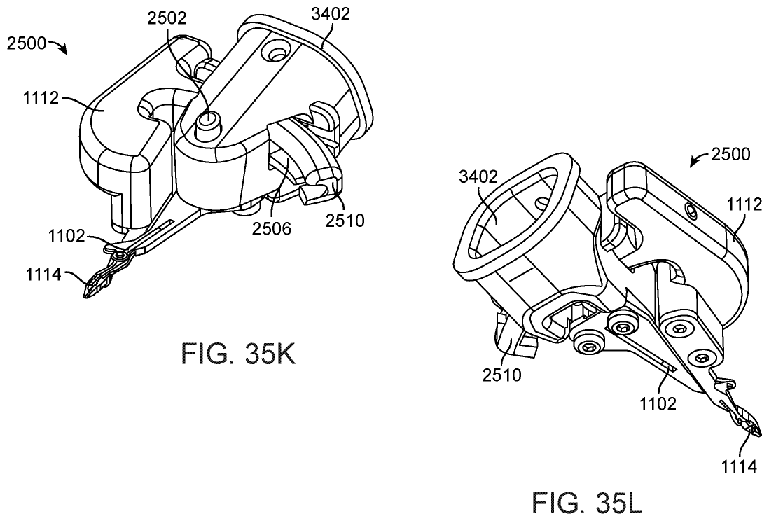

The push button delivery device 2500 can permit the surgeon to open and actuate the cutting element 1114 with a single discrete actuation of the push button 2502, reducing or eliminating reliance on continuous finger pressure to define the open extent of the cutting element 1114. The push button delivery device 2500 can provide discrete, repeatable end-states for the cutting element 1114, which can improve consistency of capsulotomy geometry across procedures and across users. Because the compression spring 2504 may drive the scissor device 1102 to a mechanically defined open position, the deployed geometry of the cutting element 1114 can be substantially the same each time the push button 2502 is actuated, regardless of user hand strength or grip variability. In some embodiments, one or more of a spring constant of the compression spring 2504, a geometry of the arced slot 2506, and a configuration of the latch opening 2508 can be selected to tune the actuation force, travel distance, and speed of deployment of the cutting element 1114. Such parameters can be adjusted to balance tactile feedback, deployment control, and user preference. Referring to FIGS. 35A-35L, the push button delivery device 2500 can be used with the same cutting elements 1114 and gimballed contact frames described above, and the push button delivery device 2500 may be attached to the handle 1110 as a delivery tip attachment as described further in paragraph [0251] below.

### Scroll Wheel Delivery Device

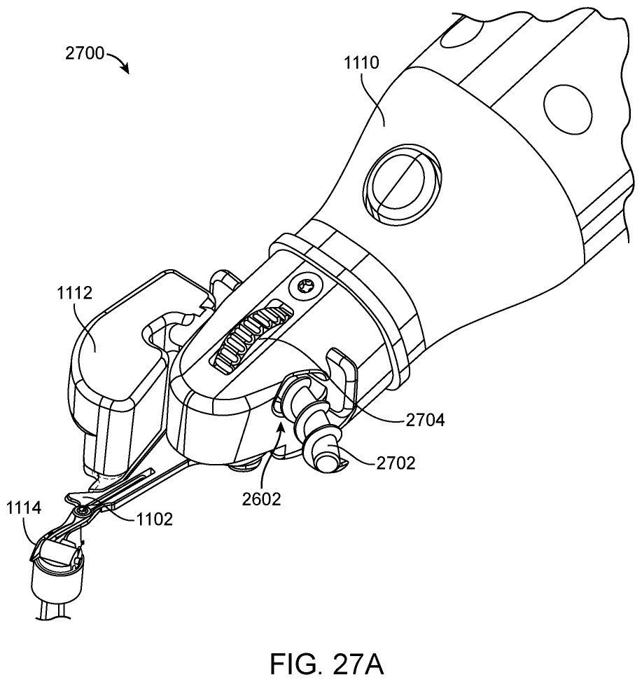

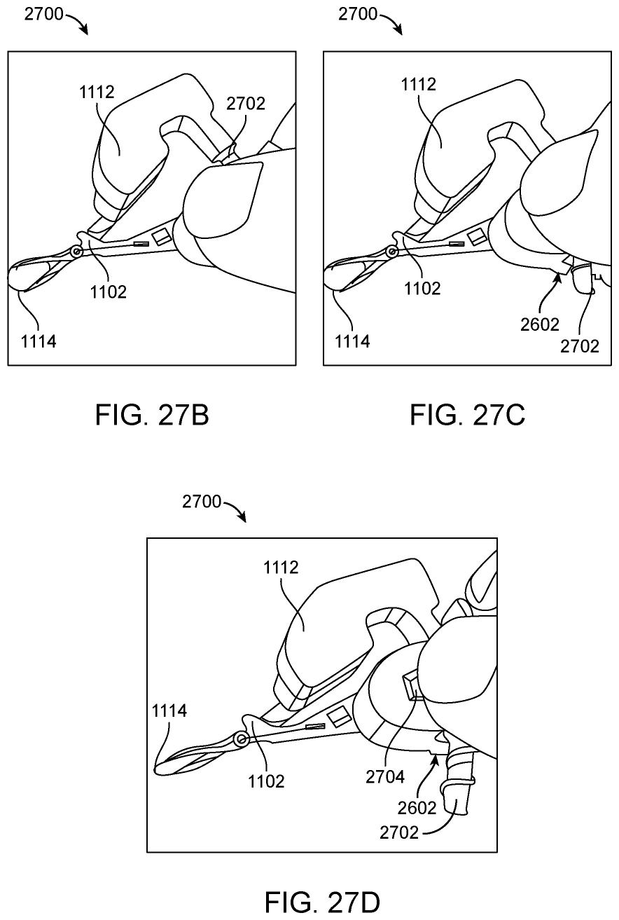

Referring to FIGS. 27A-27D, a scroll wheel delivery device 2700 is shown that can provide continuous, proportional control over deployment of the cutting element 1114. In contrast to the discrete end-state actuation provided by the one-click locking mechanism and the push button delivery device 2500 described above, the scroll wheel delivery device 2700 can permit continuous, incremental adjustment and proportional control of the cutting element 1114 configuration, thereby allowing a surgeon to position the cutting element 1114 at any intermediate point between a fully closed insertion configuration and a fully open deployed configuration. The scroll wheel delivery device 2700 comprises an arced lead screw 2702, a user-actuated scroll wheel 2704, and an end cap 2804. The arced lead screw 2702 can translate rotational motion of the user-actuated scroll wheel 2704 into linear displacement of the arms of the scissor device 1102, thereby controlling the degree of opening or closing of the cutting element 1114. In some embodiments, the scroll wheel delivery device 2700 can be compatible with use in a bagged or shrouded handpiece environment, and the user-actuated scroll wheel 2704 can be located on a disposable tip portion of the scroll wheel delivery device 2700 to permit the user to actuate the scroll wheel 2704 directly even when the handpiece is enclosed in a sterile bag or drape.

FIG. 27A shows the scroll wheel delivery device 2700 in a closed position, comprising a scissor device 1102, a handheld or palm-held section 1110, a finger-actuated device 1112, the arced lead screw 2702, the user-actuated scroll wheel 2704, and the cutting element 1114 carried at the distal end of the scissor device 1102. The scissor device 1102 can support the cutting element 1114 at a distal portion thereof. The arced lead screw 2702 can be operatively coupled to the scissor device 1102, and the user-actuated scroll wheel 2704 can be configured to drive movement of the arced lead screw 2702 relative to the handheld or palm-held section 1110. In the closed position shown in FIG. 27A, the scissor device 1102 retains the cutting element 1114 in a compact, contracted configuration suitable for insertion through a corneal incision.

Rotation of the user-actuated scroll wheel 2704 can advance or retract the arced lead screw 2702 along an arcuate path defined by the geometry of the arced lead screw 2702. The arced lead screw 2702 can engage a corresponding nut or threaded interface on the user-actuated scroll wheel 2704 such that rotation of the user-actuated scroll wheel 2704 in a first direction may advance the arced lead screw 2702 to open the scissor device 1102 and expand the cutting element 1114, and rotation of the user-actuated scroll wheel 2704 in a second, opposite direction may retract the arced lead screw 2702 to close the scissor device 1102 and contract the cutting element 1114. The movement of the arced lead screw 2702 can drive arms of the scissor device 1102 between the closed, intermediate, and open positions, thereby proportionally transitioning the cutting element 1114 between a compact insertion configuration and a deployed configuration. Because the arced lead screw 2702 provides threaded engagement with the user-actuated scroll wheel 2704, the scroll wheel delivery device 2700 can resist unintended movement of the cutting element 1114 due to external forces encountered during insertion and/or positioning within the eye, which may provide a self-locking characteristic without a separate locking element.

FIGS. 27B-27D show views of the scroll wheel delivery device 2700 in the open position (FIG. 27B), a middle position (FIG. 27C), and the closed position (FIG. 27D). An opening 2602 can be provided to accommodate travel of a distal end of the finger-actuated device 1112 across configurations as the arced lead screw 2702 advances along an arc. As the user-actuated scroll wheel 2704 is rotated, the cutting element 1114 can progressively transition between the deployed and insertion configurations. In some embodiments, the finger-actuated device 1112 can also be pressed in conjunction with rotation of the user-actuated scroll wheel 2704 to close the scissor device 1102 and expand the cutting element 1114.

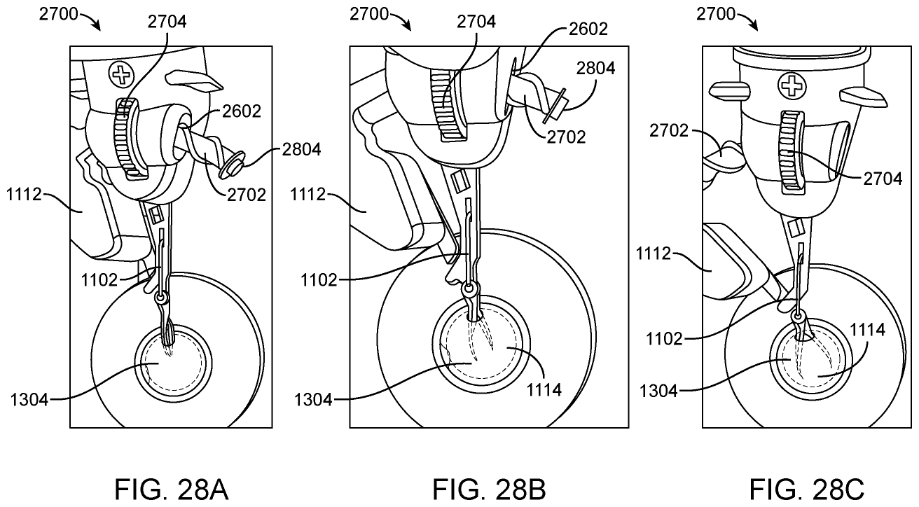

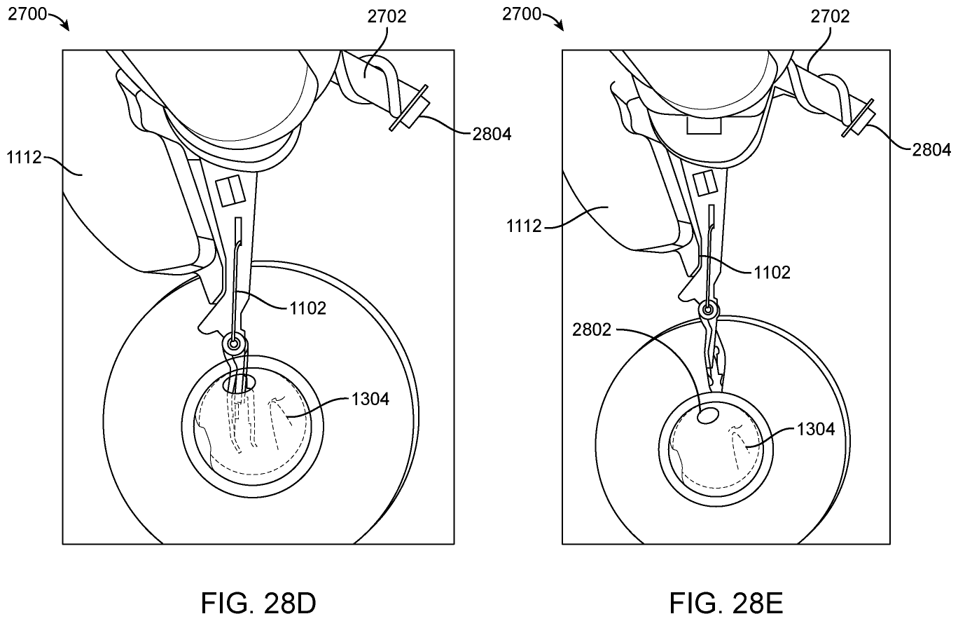

Referring to FIGS. 28A-28E, an example method of using the scroll wheel delivery device 2700 to form an opening in a capsular bag 1304 is shown, including insertion, deployment, closure, and withdrawal of the scroll wheel delivery device 2700. FIG. 28A shows the scroll wheel delivery device 2700 in the closed position being inserted through an entrance/exit opening 2802 into the capsular bag 1304, with an end cap 2804 of the arced lead screw 2702 leading the distal end during insertion. FIG. 28B shows the scroll wheel delivery device 2700 in a middle position after insertion, with the user-actuated scroll wheel 2704 partially rotated such that the cutting element 1114 begins to transition toward a deployed geometry and the end cap 2804 of the arced lead screw remains visible 2702 preventing the distal end of the arced lead screw 2702 from passing through the opening for the distal end of the finger-actuated device 2602. FIG. 28C shows the scroll wheel delivery device 2700 in the open position within the capsular bag 1304, with the cutting element 1114 expanded for capsulotomy. FIG. 28D shows the scroll wheel delivery device 2700 returned to the closed position within the capsular bag 1304 following capsulotomy, with the end cap 2804 of the arced lead screw 2702 visible. FIG. 28E shows the scroll wheel delivery device 2700 in the closed position after withdrawal from the capsular bag 1304 through the entrance/exit opening.

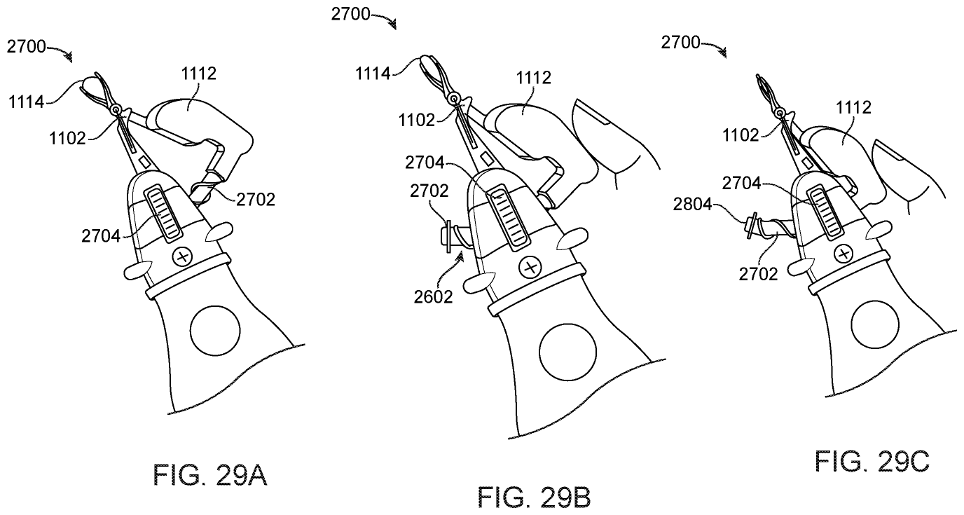

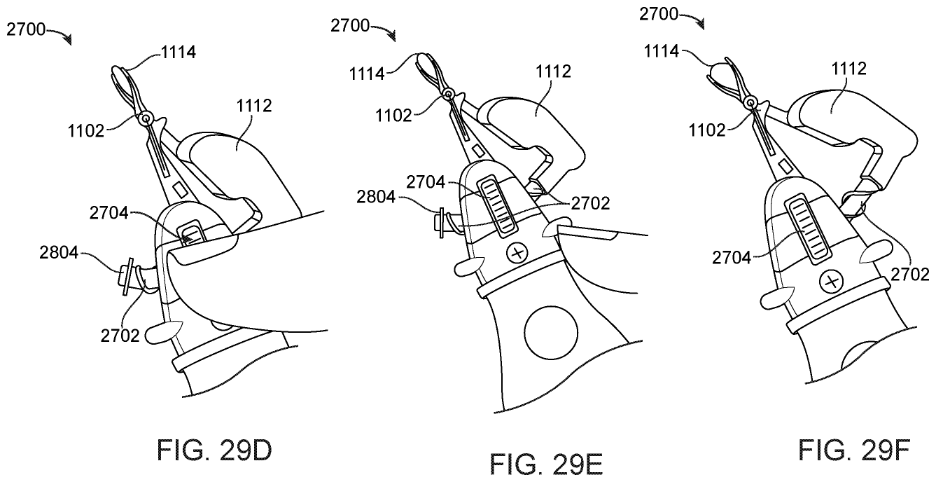

Referring to FIGS. 29A-29F, additional operational transitions of the scroll wheel delivery device 2700 are shown, illustrating incremental opening of the scissor device 1102 and the corresponding expansion of the cutting element 1114 in response to continued rotation of the user-actuated scroll wheel 2704. FIG. 29A shows the scroll wheel delivery device 2700 in the open position with the cutting element 1114 deployed. FIG. 29B shows the scroll wheel delivery device 2700 in a middle position, also illustrating an optional press on the finger-actuated device 1112 that can be used in conjunction with rotation of the user-actuated scroll wheel 2704 to expand the cutting element 1114. FIG. 29C shows the scroll wheel delivery device 2700 in the closed position with the end cap 2804 visible. FIGS. 29D and 29E show the scroll wheel delivery device 2700 in successive middle positions achieved by continued rotation of the user-actuated scroll wheel 2704 to incrementally open the scissor device 1102 and expand the cutting element 1114, with the end cap 2804 of the arced lead screw 2702 visible. FIG. 29F shows the scroll wheel delivery device 2700 in an open position after the user-actuated scroll wheel 2704 has been rotated to completely open the scissor device 1102 and fully expand the cutting element 1114.

In some embodiments, the scroll wheel delivery device 2700 can include open-position and closed-position detection features (e.g., detents, end-stop switches, capacitive or magnetic sensors, or other sensor-based detection mechanisms) configured to detect or indicate when the cutting element 1114 has reached a predefined fully open or fully closed end-position. Detection of the end-positions can be communicated to the user through haptic feedback (e.g., a tactile detent or click in rotation of the user-actuated scroll wheel 2704), audible feedback (e.g., a click or tone), and/or visual feedback (e.g., an indicator on the display 722). The feedback can confirm that the cutting element 1114 is in a known, predefined configuration before the user advances the scroll wheel delivery device 2700 into the eye, withdraws the scroll wheel delivery device 2700 from the eye, or activates an energy pulse.

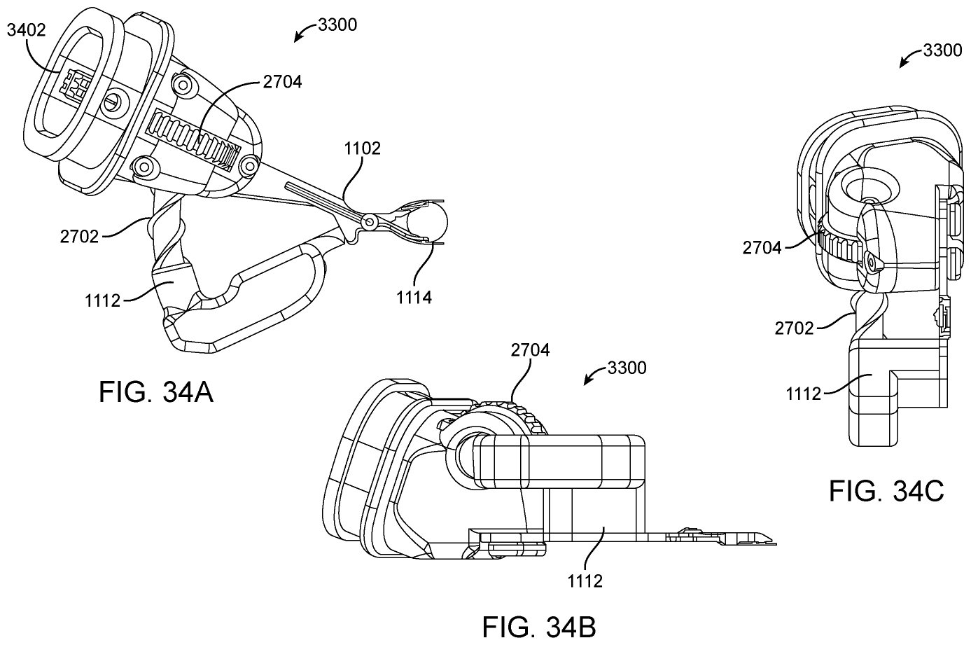

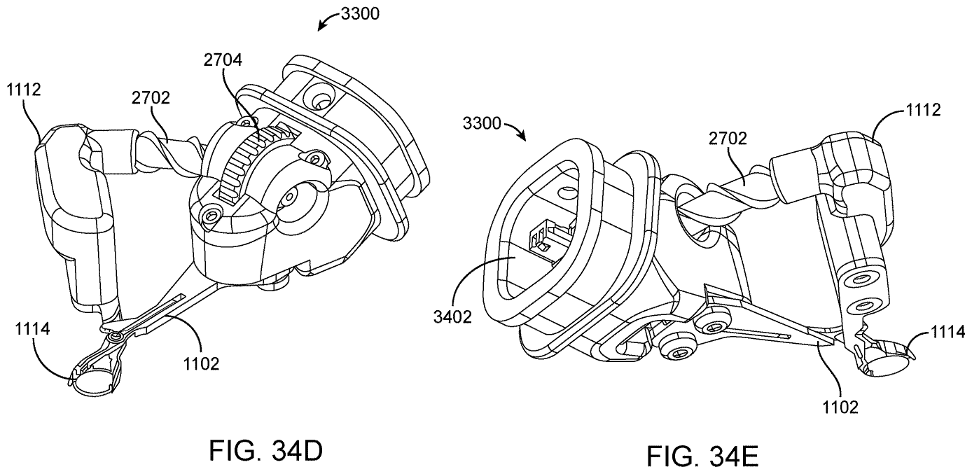

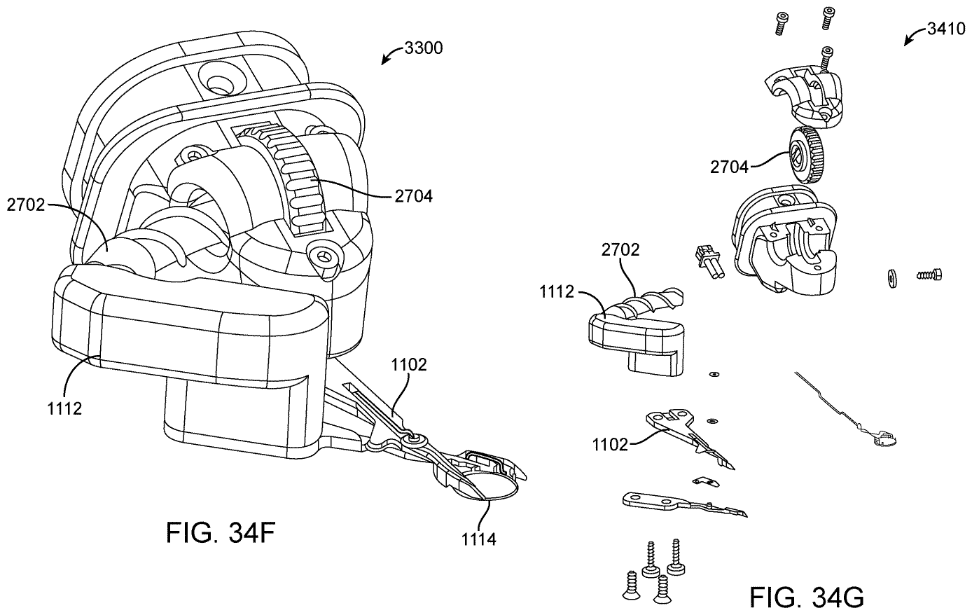

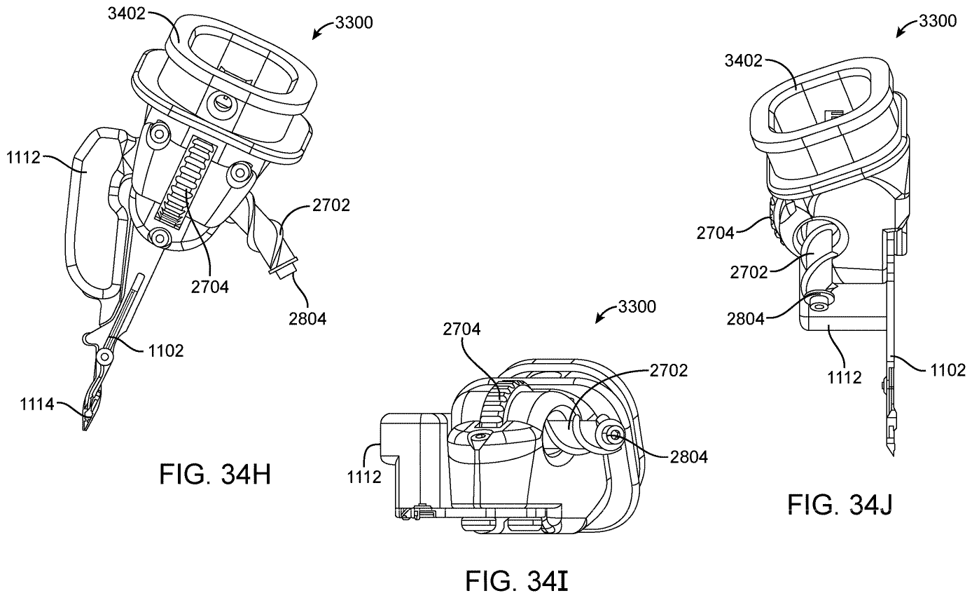

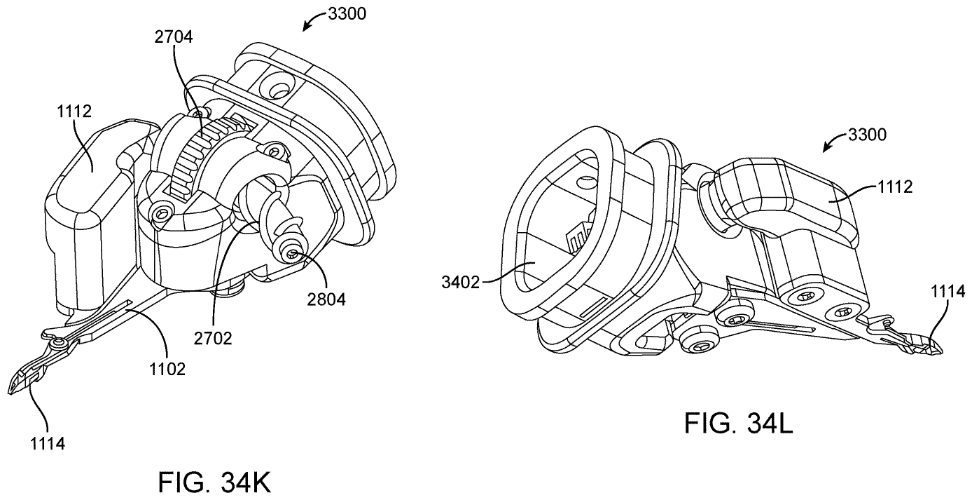

The scroll wheel delivery device 2700 can provide continuous, proportional control over the configuration of the cutting element 1114, permitting fine adjustment of the deployed geometry of the cutting element 1114 to match and accommodate patient anatomy, capsule properties, or surgical preference. While offering proportional control, the scroll wheel delivery device 2700 can also provide discrete haptic confirmation at the open and closed end-positions, thereby combining fine adjustability with procedural consistency. Referring to FIGS. 34A-34L, the scroll wheel delivery device 2700 may be used with the same cutting elements 1114, gimballed contact frames, and closed-loop conductors 902 described above, and can be attached to the handle 1110 as a scroll-wheel delivery tip attachment as described further in paragraph [0250] below.

### Base Station and Handle Assembly

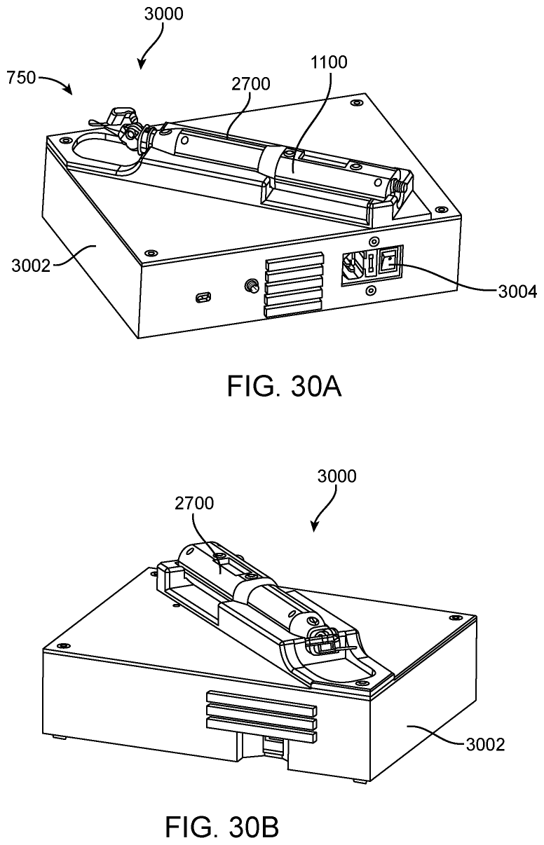

Referring to FIGS. 30A-30B, perspective views of a scroll wheel delivery device on a base station assembly 3000 are shown. FIGS. 30A-30B show the scroll wheel delivery device 2700 assembled with the handle on a base station 3002. FIG. 30A shows the scroll-wheel delivery device 2700 mounted on the base station 3002, with the handheld or palm-held section 1110 visible and the distal end 750 extending from the handheld or palm-held section 1110, also known as the handle. A base station power switch 3004 for charging can be provided on the base station 3002. The base station 3002 can serve as a charging dock and a storage receptacle for the delivery device, permitting the delivery device to be charged between procedures and securely retained when not in use. In some embodiments, the base station 3002 can be configured to charge an internal battery of the handle 1110 via electrical contacts that engage when the delivery device is seated in the base station 3002. The assembly 3000 can represent the scroll-wheel delivery device on the base station. FIG. 30B shows another perspective view of the scroll-wheel delivery device 2700 on the base station 3002, illustrating the assembly 3000.

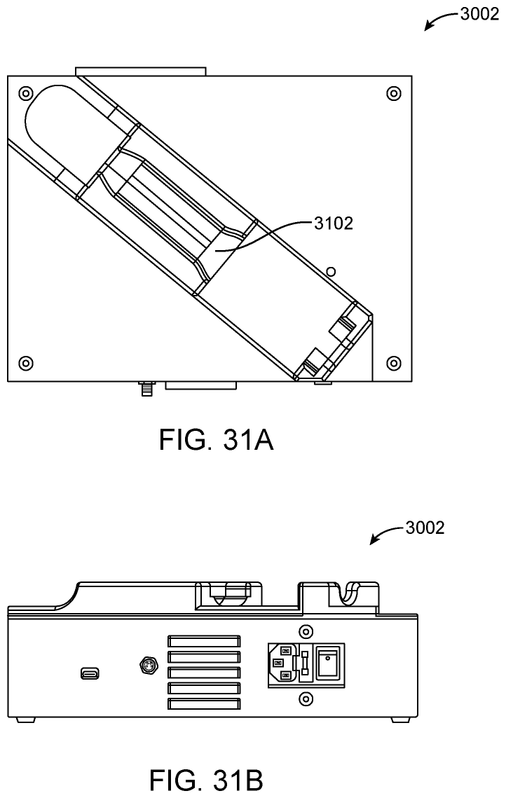

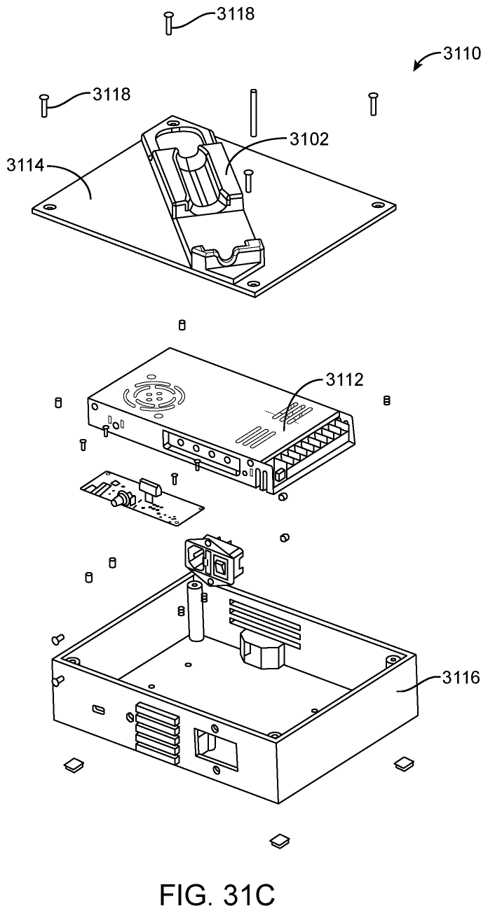

Referring to FIGS. 31A-31C, views of the base station 3002 are shown. FIGS. 31A-31B show the base station 3002 in a fully assembled state, including a top view (FIG. 31A) and a front view (FIG. 31B), showing a device receiving structure 3102 configured to hold the delivery device. The device receiving structure 3102 can be shaped to receive the handle 1110 and the distal end 750 of the delivery device, and may comprise one or more alignment features to orient the delivery device for proper electrical contact during charging. FIG. 31C shows the base station 3002 in a disassembled state 3110, illustrating internal components 3112 of the base station, the device receiving structure 3102, a top piece 3114 of the base station 3002, a bottom and side structure 3116, and fasteners 3118. The internal components 3112 can include charging circuitry, power regulation electronics, and one or more indicator lights for displaying a charging status to the user. The fasteners 3118 connect the top piece 3114 with the bottom and side structure 3116 in the assembled configuration of the base station 3002.

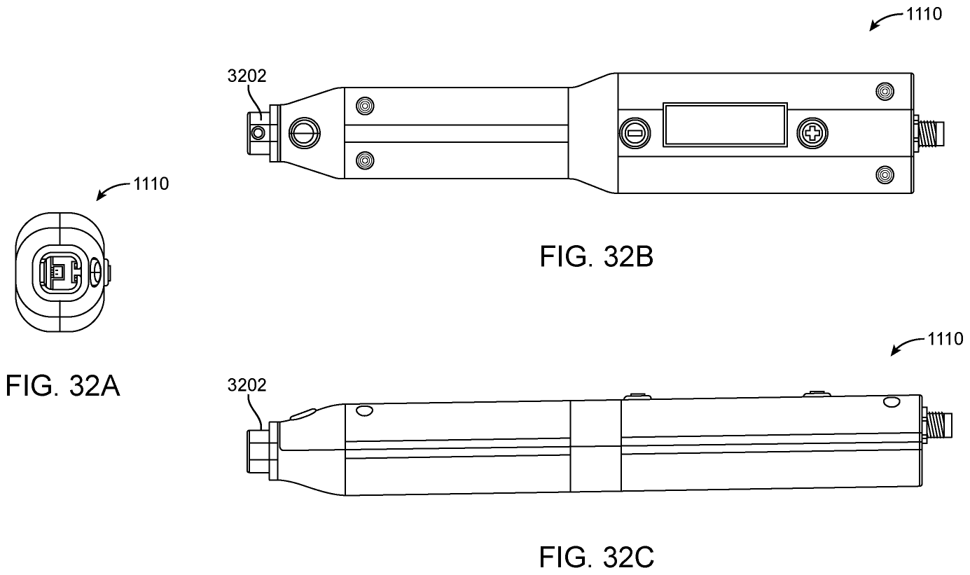

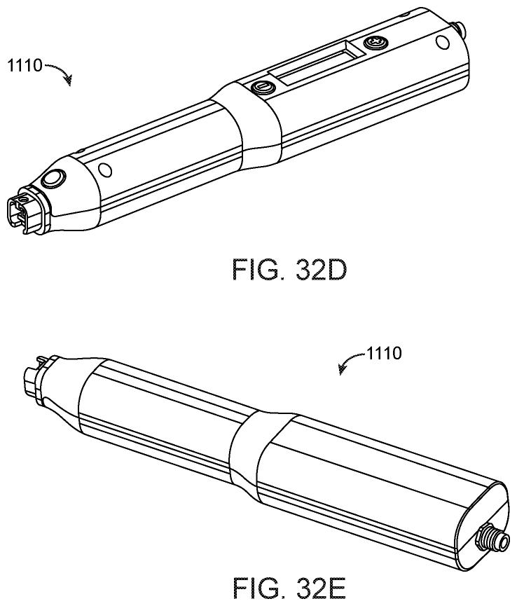

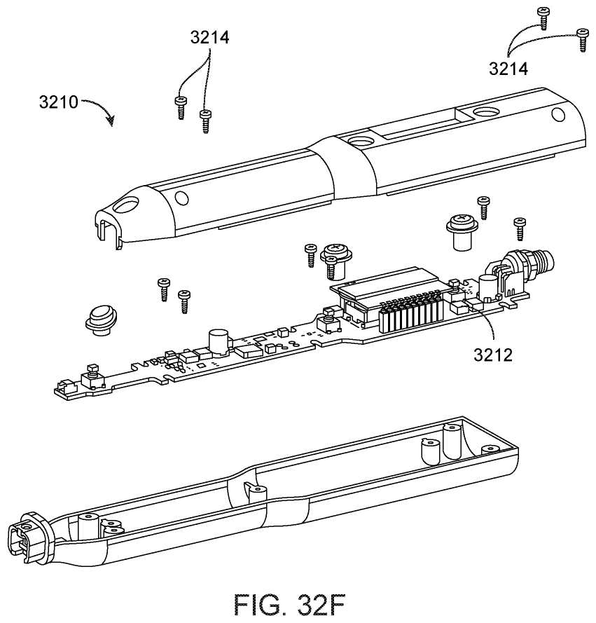

Referring to FIGS. 32A-32F, views of the handle 1110 for the delivery device are shown. FIG. 32A shows a front view of the handle 1110. FIGS. 32B-32C show side views of the handle 1110, including a distal end 3202 of the handle 1110 where a delivery tip attachment couples. The distal end 3202 of the handle 1110 can comprise an interface configured to receive and mechanically couple to one or more delivery tip attachments, such as the scroll-wheel delivery tip attachment or the spring-loaded push-button delivery tip attachment described herein. The interface can include one or more of mechanical alignment features, electrical contacts for delivering energy to the cutting element via the closed-loop conductor wire 902, and retention features for securing the delivery tip attachment during use. FIGS. 32D-32E show perspective views of the handle 1110. FIG. 32F shows a disassembled view of the handle 3210, illustrating internal components 3212 of the handle 1110 and fasteners 3214. The internal components 3212 can include one or more of a rechargeable battery, a printed circuit board (PCB) comprising control electronics and a pulse generator, a trigger button, parameter adjustment buttons, and a display. The fasteners 3214 hold the exterior of the handle 1110 together around the internal components 3212.

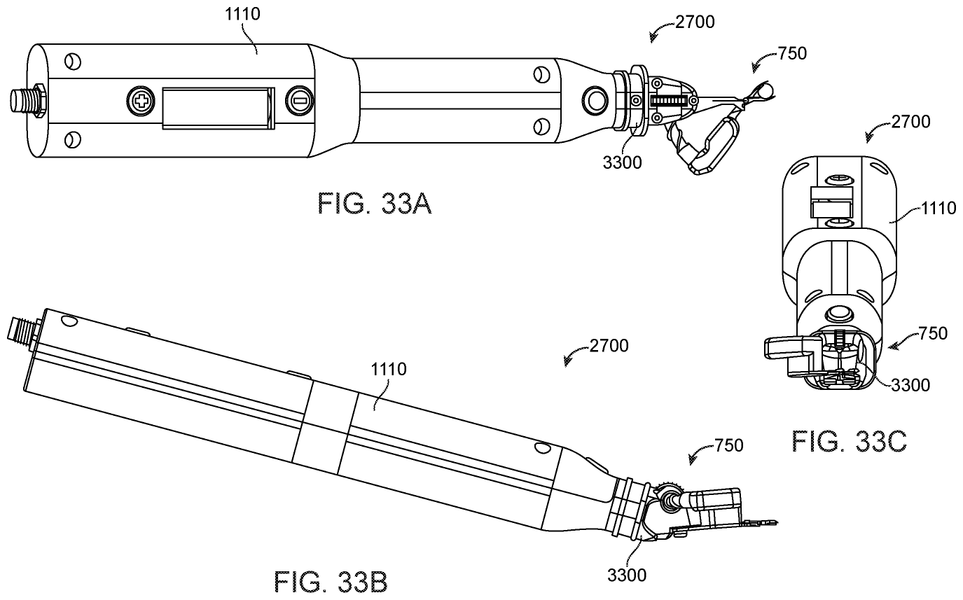

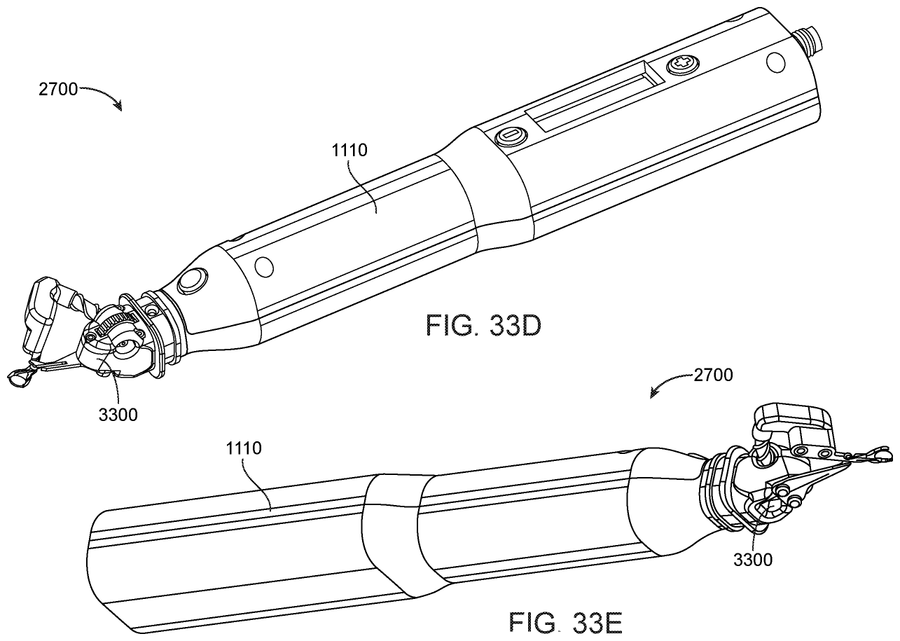

Referring to FIGS. 33A-33E, views of a fully assembled scroll-wheel delivery device comprising the handle 1110 and a scroll-wheel delivery tip attachment 3300 are shown. FIG. 33A shows a top view. FIG. 33B shows a side view. FIGS. 33C-33E show perspective views. The scroll-wheel delivery device 2700 can include the distal end 750 where the scroll-wheel delivery tip attachment 3300 interfaces with the handle 1110. The scroll-wheel delivery tip attachment 3300 can be mechanically and electronically coupled to the handle 1110 at the attachment interface, such that the handle 1110 provides power and control signals to the cutting element 1114 via the closed-loop conductor 902 routed through the scroll-wheel delivery tip attachment 3300. In this assembled configuration, a user may hold the handle 1110 and actuate the user-actuated scroll wheel 2704 on the scroll-wheel delivery tip attachment 3300 to control deployment of the cutting element 1114, while using the trigger button on the handle 1110 to initiate energy delivery to the cutting element 1114.

Referring to FIGS. 34A-34L, views of the scroll-wheel delivery tip attachment 3300 are shown in open and closed configurations, and in assembled and disassembled states. FIGS. 34A-34G show the tip attachment in an open configuration: FIG. 34A shows a top view; FIG. 34B shows a side view; FIGS. 34C-34F show perspective views; and FIG. 34G shows a disassembled view of the scroll-wheel delivery tip attachment 3410. FIGS. 34H-34L show the tip attachment in a closed configuration: FIG. 34H shows a top view; FIG. 34J shows a side view; and FIGS. 34I, 34K-34L show perspective views. The tip attachment 3300 can include the scissor device 1102, the finger-actuated device 1112, the cutting element 1114, the arced lead screw 2702, the user-actuated scroll wheel 2704, an end cap 2804, and an attachment interface 3402 for the handle 1110. The attachment interface 3402 can comprise a mechanical coupling (e.g., a bayonet connector, a snap-fit latch, or a threaded interface) and one or more electrical contacts that may engage corresponding contacts on the handle 1110 when the tip attachment 3300 is attached. The scroll-wheel delivery tip attachment 3300 may be provided as a disposable, sterile component that can be replaced between procedures while the handle 1110 is reused.

Referring to FIGS. 35A-35L, views of a spring-loaded push-button delivery tip attachment are shown in open and closed configurations. FIGS. 35A-35G show the spring-loaded tip attachment in an open configuration: FIG. 35A shows a top view; FIGS. 35B-35C show side views; FIGS. 35D-35F show perspective views; and FIG. 35G shows a disassembled view of the spring-loaded delivery tip attachment 3500. FIGS. 35H-35L show the spring-loaded tip attachment in a closed configuration: FIG. 35H shows a top view; FIGS. 35I-35J show side views; and FIGS. 35K-35L show perspective views. The spring-loaded tip attachment can include the push button delivery device 2500, the scissor device 1102, the finger-actuated device 1112, the cutting element 1114, the push button 2502, the compression spring 2504, the arced slot 2506, the latch opening 2508, the arm 2510 of the finger-actuated device, an attachment interface 3402 for the handle, and an opening 2602 for the distal end of the finger-actuated device. The spring-loaded delivery tip attachment 3500 can interface with the handle 1110 via the attachment interface 3402 in the same manner as the scroll-wheel delivery tip attachment 3300, such that either tip attachment may be selected by the user and attached to the same handle 1110 depending on the desired actuation mechanism for a given procedure.

### Methods

Provided herein are methods for using the devices and systems described herein to treat cataracts. The methods can comprise capsulotomy. The methods can comprise capsulorhexis.

A user (e.g., physician, assistant, surgeon, or other medical professional) can select a disposable tip with a contact frame of a desired perimeter (e.g., 5 mm). The tip can be attached to a distal end of the handpiece or robotic adapter, automatically connecting electrical contacts.

The portable controller can be powered on. The user can select a profile corresponding to the tip size and targeted tissue. The controller may confirm the tip identity via mechanical keying or electronic tagging.

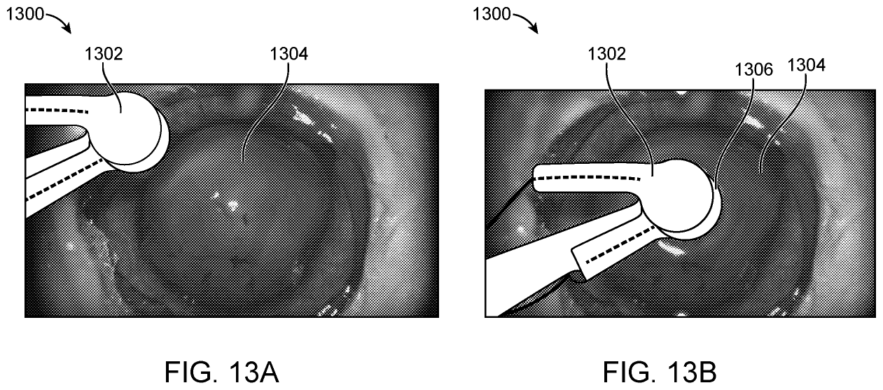

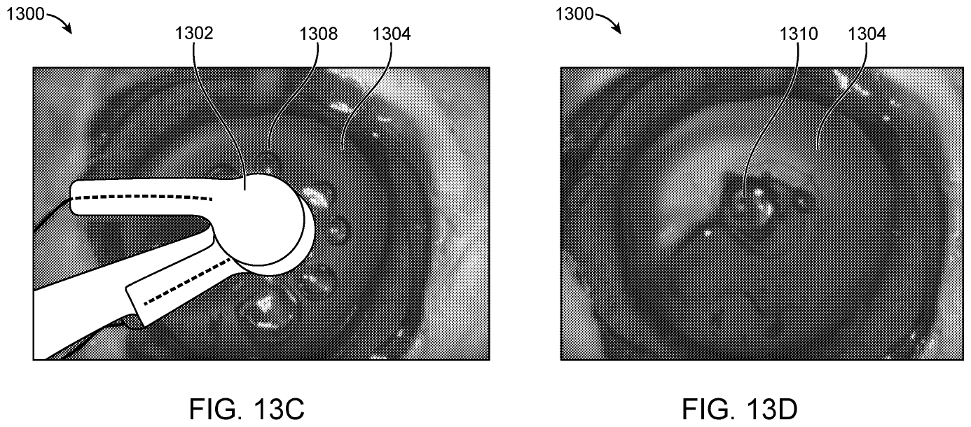

With the frame (e.g., cutting element) in an insertion configuration (e.g., the deployment mechanisms described above, including but not limited to elongation or folding into a tubular or elliptical shape), the distal assembly can be introduced through a corneal incision into the anterior chamber. FIG. 13A shows a capsulotomy and capsulorhexis method 1300 comprising a capsular bag 1304 and a device 1302 as visible through a corneal incision.

The delivery device can be actuated to move the contact frame (e.g., cutting element) into a deployed configuration (e.g., a more rounded configuration ready for use). As a non-limiting example, the scissor-type delivery device would bring the sides of the scissors together to round out the cutting element, and the half-tube based device would bring together the shrink tubing to stop stretching the cutting element and round the cutting element out. Other types of delivery devices may have other actuation based on the cutting element's deployment mechanism. Once deployed, the heating element can return to a shape memory geometry of the heating element with a predetermined perimeter in the absence of forces by the delivery devices or with the assistance of the delivery devices. The deployment can be manual or automatic. In some cases, a controller in or separate from the power source can prevent heating or activating of the heating element unless and until the heating element is confirmed to be deployed in the anterior chamber. Confirmation of deployment can be done visually, by measuring resistance, or by other methods.

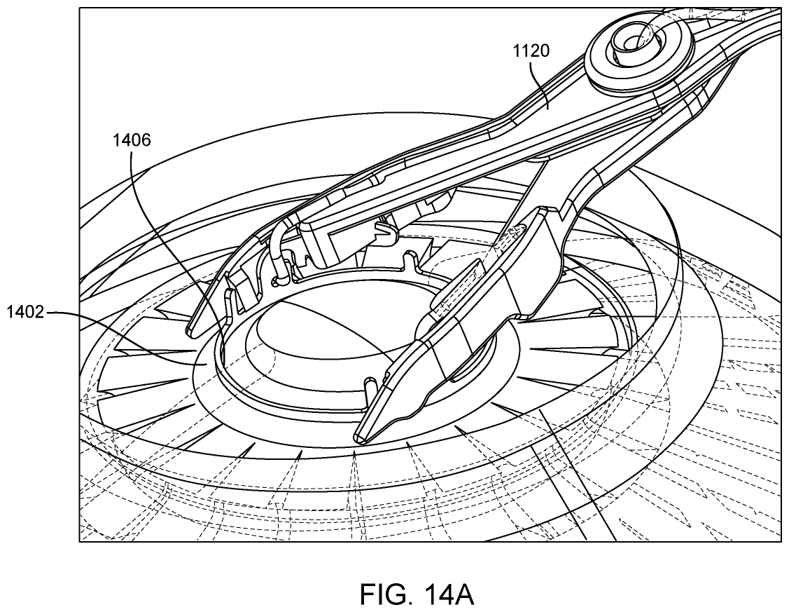

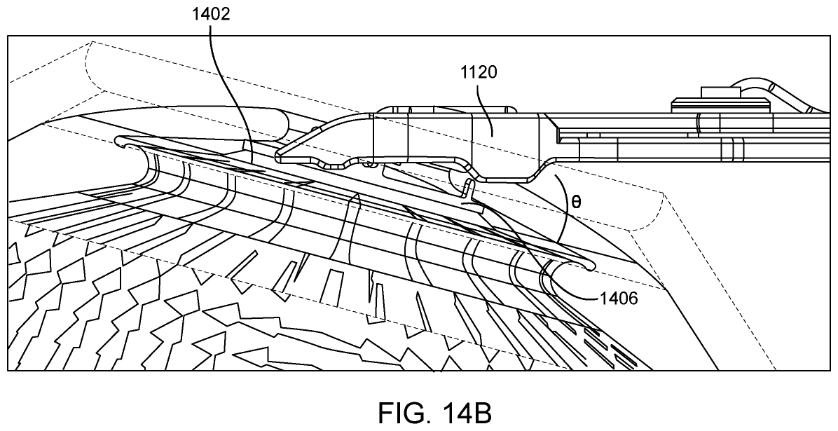

The surgeon or robotic arm can advance to position the frame so that the frame approaches the anterior capsule. In some cases, the cutting element can be seated substantially along the entire perimeter of the anterior lens capsule without tilting (e.g., depending on the axis at which the delivery device is introduced into the eye, or the bending of the distal tip of the delivery device relative to the housing as shown in FIG. 7A). FIG. 13B shows contact between the capsular bag 1304 and the device 1302, where the contact can be visualized as a pressure-indented ring 1306. In some cases, contact between the frame and capsule can cause the frame to tilt (e.g., via the gimbal) and seat against the capsule along substantially the frame's entire perimeter, as shown with the gimbal of FIGS. 14A-14B using scissors 1120, ring 1406, and lens 1402. Ring 1406 can be any of the rings described above. Although the scissor 1120 device is used for the example in FIGS. 14A-14B, the ring can likewise be used with the other delivery devices described above.

Although the particular example of FIG. 14B has a 0 of about 15 degrees, the gimbal angle can be from about 0 degrees to about 80 degrees. The gimbal angle can be from about 0 degrees to about 10 degrees, about 0 degrees to about 20 degrees, about 0 degrees to about 30 degrees, about 0 degrees to about 40 degrees, about 0 degrees to about 50 degrees, about 0 degrees to about 60 degrees, about 0 degrees to about 70 degrees, about 0 degrees to about 80 degrees, about 10 degrees to about 20 degrees, about 10 degrees to about 30 degrees, about 10 degrees to about 40 degrees, about 10 degrees to about 50 degrees, about 10 degrees to about 60 degrees, about 10 degrees to about 70 degrees, about 10 degrees to about 80 degrees, about 20 degrees to about 30 degrees, about 20 degrees to about 40 degrees, about 20 degrees to about 50 degrees, about 20 degrees to about 60 degrees, about 20 degrees to about 70 degrees, about 20 degrees to about 80 degrees, about 30 degrees to about 40 degrees, about 30 degrees to about 50 degrees, about 30 degrees to about 60 degrees, about 30 degrees to about 70 degrees, about 30 degrees to about 80 degrees, about 40 degrees to about 50 degrees, about 40 degrees to about 60 degrees, about 40 degrees to about 70 degrees, about 40 degrees to about 80 degrees, about 50 degrees to about 60 degrees, about 50 degrees to about 70 degrees, about 50 degrees to about 80 degrees, about 60 degrees to about 70 degrees, about 60 degrees to about 80 degrees, or about 70 degrees to about 80 degrees. The gimbal angle can be about 0 degrees, about 10 degrees, about 20 degrees, about 30 degrees, about 40 degrees, about 50 degrees, about 60 degrees, about 70 degrees, or about 80 degrees. The gimbal angle can be at least about 0 degrees, about 10 degrees, about 20 degrees, about 30 degrees, about 40 degrees, about 50 degrees, about 60 degrees, or about 70 degrees. The gimbal angle can be at most about 10 degrees, about 20 degrees, about 30 degrees, about 40 degrees, about 50 degrees, about 60 degrees, about 70 degrees, or about 80 degrees.

The user or robotic controller can command the portable controller to deliver one or more ultrashort energy pulses to the closed-loop conductor (e.g., the wires described herein). The pulse can be a direct current pulse. During the delivery of energy, the capsular bag may form bubbles as shown in FIG. 13C. FIG. 13C shows device 1302 forming bubbles 1308 on capsular bag 1304. The capsule can then be thermally ablated along the frame path, forming an opening corresponding to the frame geometry. FIG. 13D shows opening 1310 in capsular bag 1304.

The frame can then be returned to its insertion configuration. The distal assembly can be withdrawn from the chamber. This can be the end of capsulorhexis. The capsulotomy procedure can continue with lens removal and IOL implantation. Such a method can be used for anterior capsulotomy for cataract surgery, posterior capsulotomy performed intraoperatively, capsular relaxing incision for capsular fibrosis, iridotomy, iridectomy, pediatric elastic capsules, intumescent cataracts, creating a glaucoma outflow channel, or other ocular operations.

As a non-limiting example, in use, a surgeon can select a distal tip with a heated element that is sized and/or shaped to form a hole with a corresponding size and/or shape. The tip can be selected from a variety of tips or a tip kit, as described above. Each tip may comprise a heating element and a closed-loop conductor. The tip may comprise a gimballed joint structure. For example, if the surgeon desires to form a circular hole with a 6-millimeter diameter, the surgeon can select a distal tip with a heated element that has a circular shape with a 6-millimeter diameter. The surgeon can attach the distal tip to the handle. The surgeon can bring the heated element into contact with or proximate an anterior portion of the capsular bag. The surgeon can interact with (e.g., push) the user interface, which can cause the heated element to be heated to a temperature of 60-150 °C or others. As described herein, direct electrical current can be delivered from the handle (e.g., power source, battery, and/or wired connection) to the heated element to raise a temperature of the heated element, which can be via resistive heating. In some cases, a pulse (e.g., pulse modulation) of direct electrical current can be delivered to the heated element, which can rapidly heat the heated element. The heat from the heated element can be conducted to the tissue of the capsular bag that is in contact with or proximate the heated element, which can sever the tissue of the capsular bag to form the hole. In some cases, the surgeon may not move the heated element across the surface of the capsular bag because the heated element has a shape and size (e.g., circular shape with a target diameter) that, when heated, can immediately form a hole of a corresponding shape and size in the capsular bag. In some cases, the surgeon may move the heated element across the surface of the capsular bag to form the hole. The severed portion of the capsular bag can be removed (e.g., removed with forceps). With pulses of direct electrical current (e.g., pulse modulation), the heating and cooling of the heated element can be rapid and/or controlled without dissipation to nearby regions of the capsular bag and/or other ocular features. With the hole formed, the natural lens within the capsular bag can be accessed. The natural lens can be removed and an IOL can be implanted in the emptied capsular bag through the formed hole.

FIG. 15 illustrates the results of a bench-top cadaver test using a thermal device as described herein using the methods described herein to form a hole in a capsular bag. As shown, the capsular bag 1502 includes a hole 1506 formed therein to provide access to the natural lens 1508 within the capsular bag 1502. The periphery 1504 of the hole 1506 can be a clean severance. For example, the capsular bag 1502 can be locally carbonized and/or ablated at the site of the heated element 108 (e.g., at the periphery 1504), which can form a clean severance that may not be as susceptible to tearing and/or may not rely upon tangential or radial stress to separate tissue.

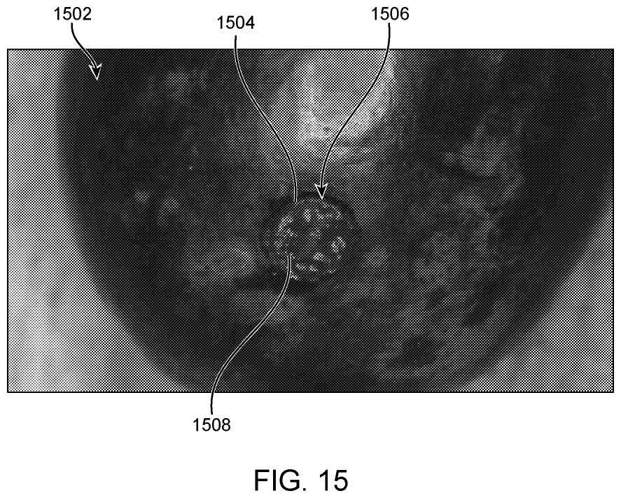

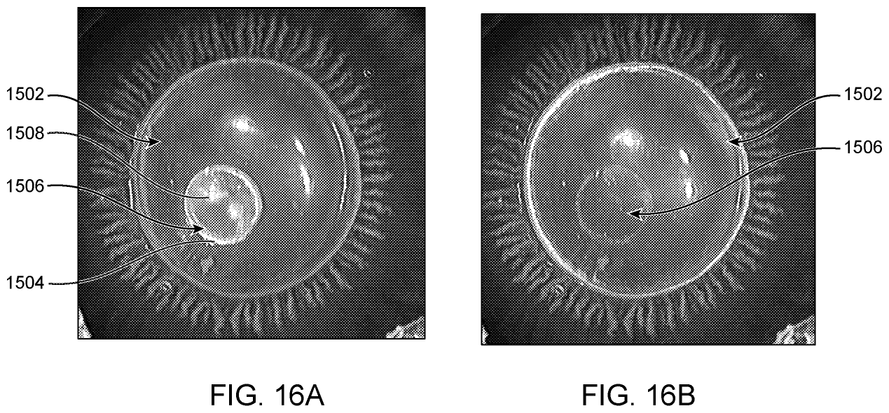

FIGS. 16A-16B illustrate the results of a bench-top cadaver test using a thermal device as described herein using the methods described herein to form a hole in a capsular bag with FIG. 16A showing an anterior view and FIG. 16B showing a posterior view. As shown, the capsular bag 1502 includes a hole 1506 with a periphery 1504 formed therein to provide access to the natural lens 1508 within the capsular bag 1502.

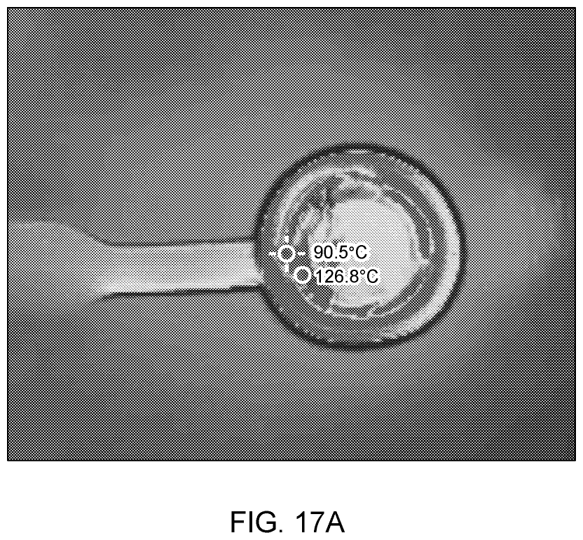

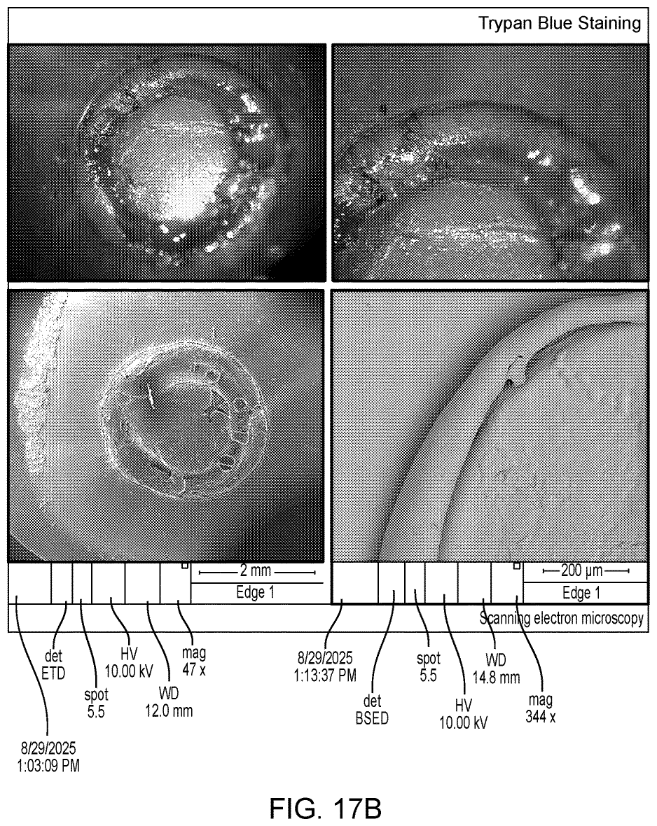

FIGS. 17A-17B show additional capsular bag holes made with the devices disclosed herein, with images captured via thermal infrared imaging (FIG. 17A), Trypan Blue staining, and scanning electron microscopy (FIG. 17B).

The methods described herein can be used in human or veterinary ophthalmic surgery. The methods described herein can also be used in non-ophthalmic procedures comprising one or more of tympanostomy, dura opening, dermatologic excision, or endoscopic mucosal resection.

### Performance and Testing Results

In an example surgical demonstration, a delivery device was inserted through a small corneal incision into an ex vivo bovine eye with the scissor device 1102 or the forceps-style device 1102 in a closed configuration. Upon insertion, the cutting element 1114 was expanded within the anterior chamber to contact the anterior capsule, and the gimballed contact frame self-aligned against the capsular surface via the pivoting frame to ensure uniform contact without suction or vacuum. An energy pulse was then delivered, and the capsulotomy was performed. Following the capsulotomy, the delivery device was returned to the closed configuration and withdrawn through the incision.

The devices, systems, and methods disclosed herein have been characterized in ex vivo and in vivo studies to assess biomechanical performance, thermal safety, edge morphology, and postoperative healing. The studies described in this section employed pig eyes, rabbit eyes, and human eyes obtained from suitable preclinical sources, and compared capsulotomies created using the devices disclosed herein (referred to herein as “Atlas one-click capsulotomy” or “Atlas OCC”) against capsulotomies created using manual continuous curvilinear capsulorhexis (referred to herein as “manual CCC”) as a comparator. In a representative study, capsulotomies were performed on 41 pig eyes, 4 human eyes, and 6 rabbit eyes. The studies evaluated multiple performance endpoints, including peak tear force, displacement at peak force, thermal dispersion in the anterior chamber, edge morphology by scanning electron microscopy, and in vivo healing and capsular stability over a postoperative follow-up period.

The capsulotomy edge created by the devices and methods disclosed herein can exhibit a greater peak tear force than a manually created continuous curvilinear capsulorhexis (CCC). Peak tear force can represent the maximum tensile force that the capsulotomy edge can withstand before tear initiation, and a higher peak force may indicate a more durable and tear-resistant capsulotomy edge. In ex vivo testing, the mean peak tear force of an Atlas OCC capsulotomy was approximately 0.183 N compared to approximately 0.150 N for manual CCC, with the difference being statistically significant at approximately p=0.0357. The capsulotomy edge can also exhibit a greater displacement at peak force than manual CCC, indicating greater extensibility and toughness prior to failure. Displacement at peak force can represent the distance that the capsulotomy edge can stretch under tension before tear propagation begins, and a greater displacement may indicate that the capsulotomy edge can deform further under surgical stress without tearing. In ex vivo testing, the mean displacement at peak force was approximately 9.947 mm for Atlas OCC compared to approximately 7.366 mm for manual CCC, with the difference being statistically significant at approximately p=0.0023.

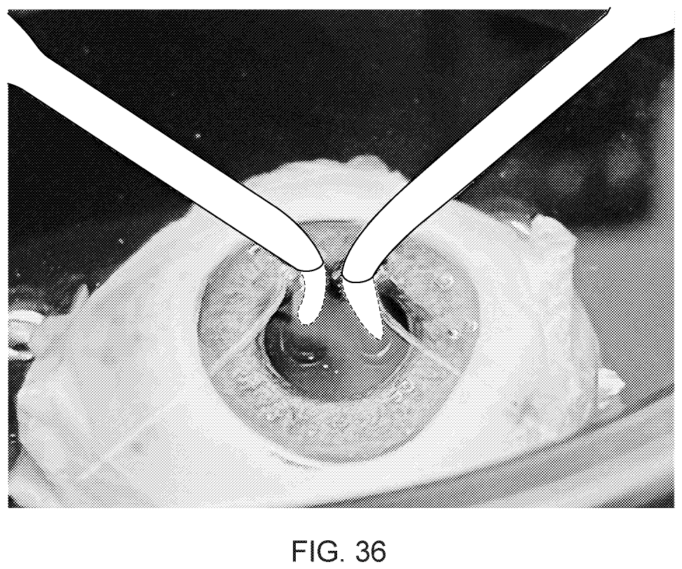

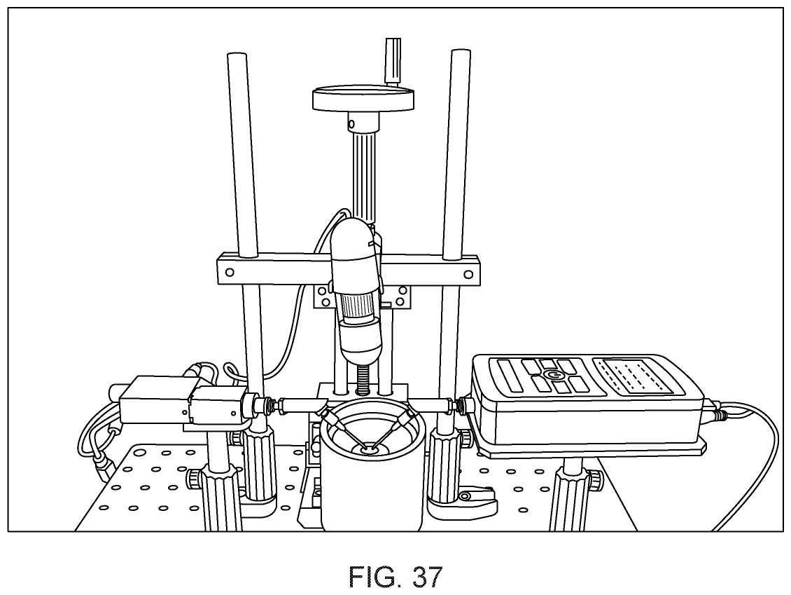

Referring to FIG. 36, a representative capsulotomy specimen and eye preparation is shown, illustrating the specimen preparation used in biomechanical testing with the specimen mounted for testing. Referring to FIG. 37, a mechanical testing apparatus used to quantify tear behavior of the capsulotomy edge under controlled tensile loading is shown. In the testing apparatus, the eye was suspended in a floating submerged chamber and engaged with opposing hooks or retractors at the capsulotomy edge, such that the capsular tissue was loaded in tension while floating, thereby isolating the measured force to that of the capsular bag without confounding fixation forces from fixation of the eye. The floating submerged chamber can reduce artifact forces that might otherwise arise from rigid fixation of the globe, permitting the measured force-distance data to reflect the intrinsic mechanical properties of the capsulotomy edge. The hooks or retractors can be advanced at a controlled displacement rate to produce force-distance curves from which peak tear force and displacement at peak force may be extracted.

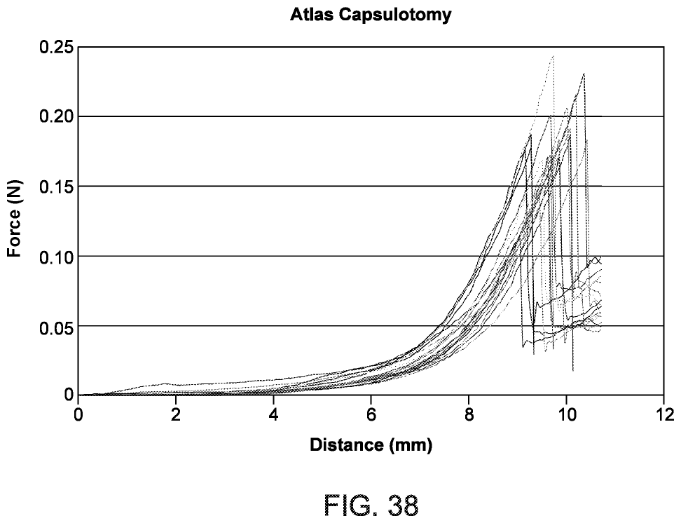

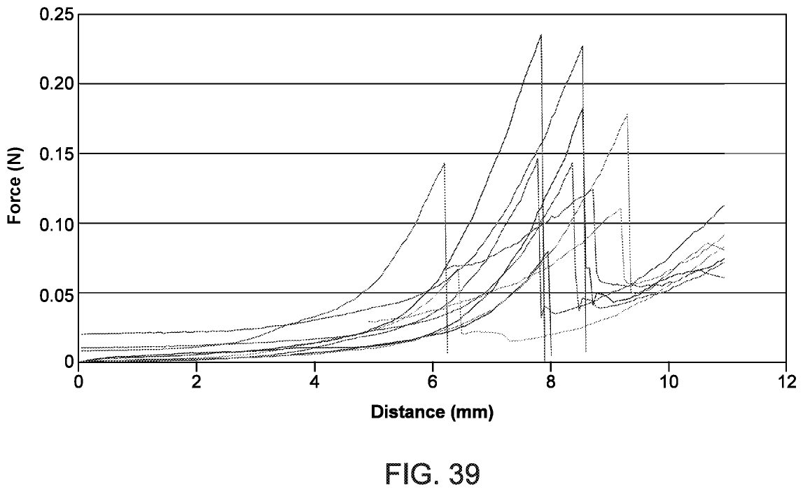

Referring to FIG. 38, an overlay plot of force-distance curves for individual capsulotomy specimens created using the devices disclosed herein (e.g., Atlas OCC) is shown. The traces show force rising progressively with increasing displacement until tear initiation, with most samples reaching peak force at relatively higher force values and at relatively greater displacement before failure. The clustering of Atlas OCC traces at higher force and higher displacement values may indicate reproducibility of the capsulotomy edge quality across samples. Referring to FIG. 39, a corresponding overlay plot of force-distance curves for individual specimens created using manual CCC is shown. Compared to the Atlas OCC samples, the manual CCC samples demonstrate earlier failure and broader sample-to-sample variability, with several traces reaching peak force at shorter displacement than the Atlas OCC traces. The broader variability in the manual CCC traces may reflect the skill-dependent nature of the manual technique and the influence of variable capsular tearing patterns on edge quality.

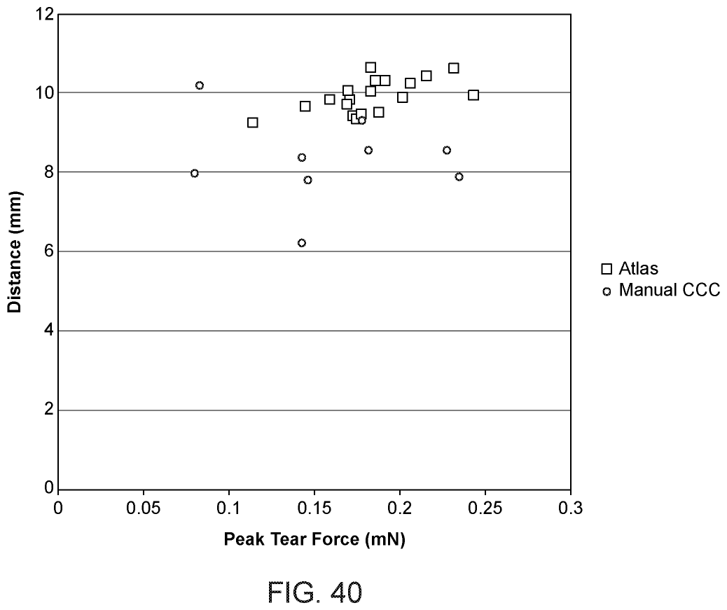

Referring to FIG. 40, a scatter plot comparing peak tear force versus displacement at peak force for both the Atlas OCC and manual CCC samples is shown. The Atlas OCC samples cluster at higher peak tear force and at greater displacement before failure than the manual CCC samples, confirming both greater strength and greater extensibility of the capsulotomy edges produced by the devices disclosed herein at both biomechanical endpoints simultaneously.

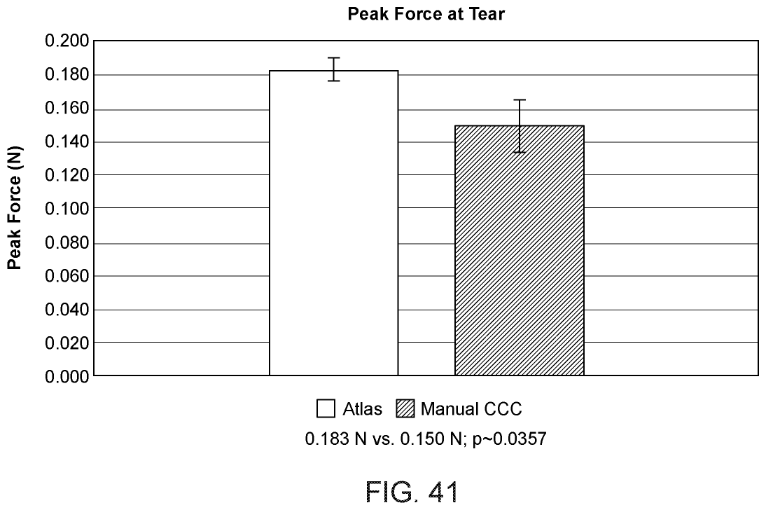

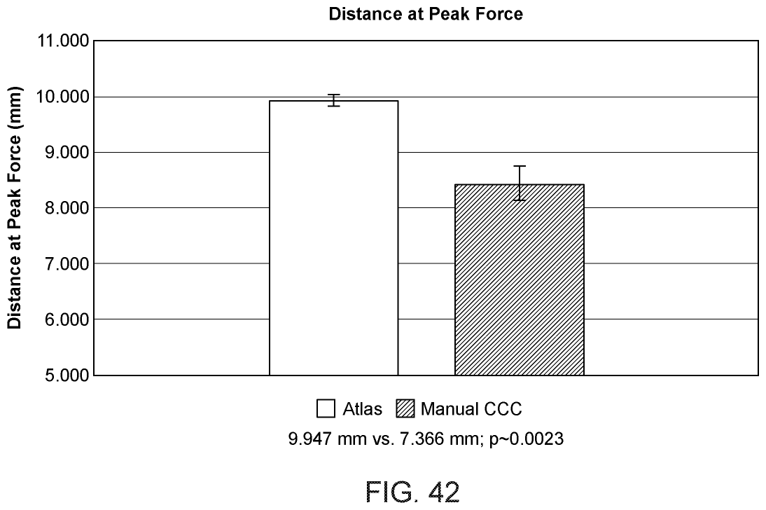

Referring to FIG. 41, a summary bar graph of mean peak tear force for the Atlas OCC and manual CCC groups is shown. Referring to FIG. 42, a corresponding summary bar graph of mean displacement at peak force for the Atlas OCC and manual CCC groups is shown. In the representative study, the mean peak tear force was approximately 0.183 N for the Atlas OCC samples versus approximately 0.150 N for the manual CCC samples, with the difference being statistically significant at approximately p=0.0357. The mean displacement at peak force was approximately 9.947 mm for the Atlas OCC samples versus approximately 7.366 mm for the manual CCC samples, with the difference being statistically significant at approximately p=0.0023. A higher peak tear force indicates that the capsulotomy edge is harder to rip, and a greater distance at peak force indicates that the capsulotomy edge can deform further before failure, resulting in a tougher, safer, and more forgiving capsulotomy. A capsulotomy edge that requires more force to tear is less likely to radialize during hydrodissection, nucleus manipulation, cortex removal, or IOL insertion, providing a more durable and more forgiving capsular opening under real surgical stress.

Without intending to be bound by theory, the improved tear resistance and toughness exhibited by capsulotomies created with the devices disclosed herein can be attributable to the repeatability of capsulotomy diameter and circularity achieved by the frame-dictated geometry of the disclosed devices. Because the geometry of the capsulotomy is dictated by the contour of the contact frame rather than by manual tearing, the resulting edge can be more uniform and have fewer discontinuities, serrations, or stress concentration points than a manually created CCC edge. A more uniform edge can in turn require greater force to initiate tearing and can deform further before tear propagation. The repeatability of capsulotomy diameter and circularity via the Atlas OCC, in comparison to manual CCC, and the resulting edge quality and repeatability are contributors to the observed stronger tear force and longer distance to peak force. The improved edge quality can reduce the likelihood of radialization of the capsulotomy during subsequent surgical steps such as hydrodissection, nucleus manipulation, cortex removal, or IOL insertion.

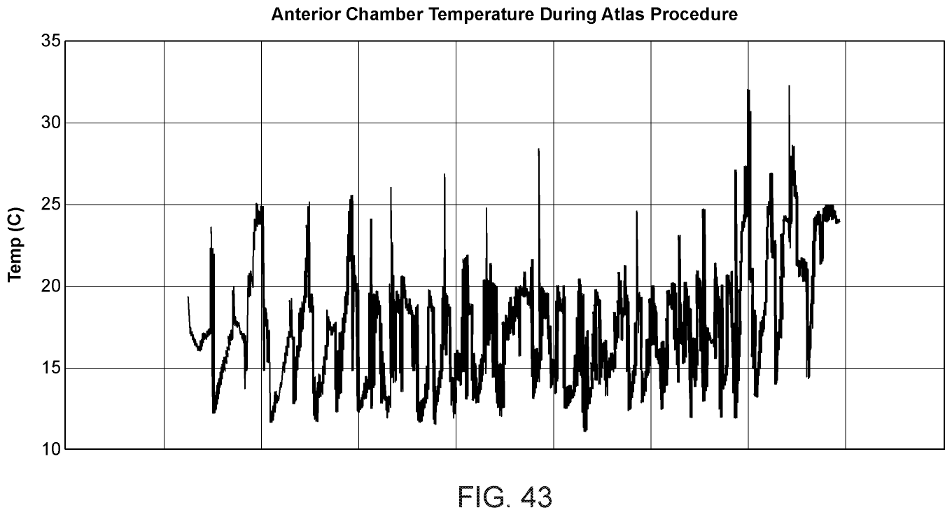

Referring to FIG. 43, a thermal safety trace recording anterior chamber temperature during sequential capsulotomies performed using the devices disclosed herein is shown. The thermal dispersion testing shows the full anterior chamber temperature trace during sequential procedures. Throughout the testing, measured anterior chamber temperatures remained below approximately 35 degrees Celsius, which is below a stated upper safety limit for thermal exposure of the anterior chamber. The thermal safety data support the local thermal safety of the pulsed ablation approach described herein and indicate that energy delivered to the cutting element can be confined to the targeted capsular tissue with limited collateral heating of surrounding ocular structures. The confinement of thermal energy to the cutting element may be attributable to the low thermal mass of the cutting element, which can heat and cool rapidly during the ultrashort pulse, and to the brief pulse duration, which may limit the time available for thermal conduction from the cutting element to the surrounding anterior chamber fluid and adjacent ocular structures such as the corneal endothelium, the iris, and the posterior capsule.

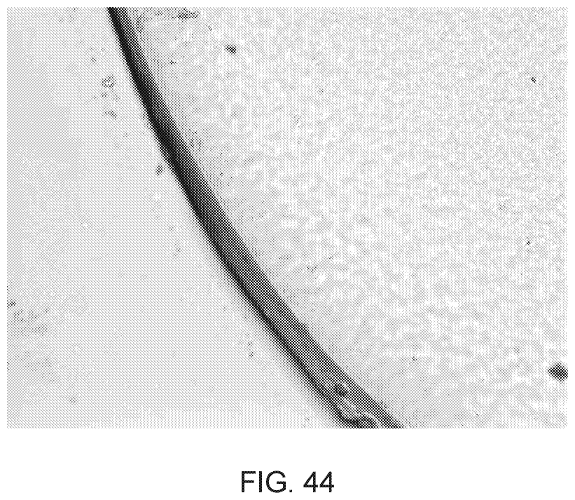

Referring to FIG. 44, an optical micrograph of excised capsular flaps created using the devices disclosed herein is shown, illustrating the substantially circular geometry of the capsulotomy and the gross edge quality of the resulting opening. The flap edge produced by the Atlas OCC appears clean and continuous, without visible serrations or discontinuities, and is comparable in gross appearance to a flap excised by manual CCC, with a clean finish without any serrations or discontinuous or discrete regions.

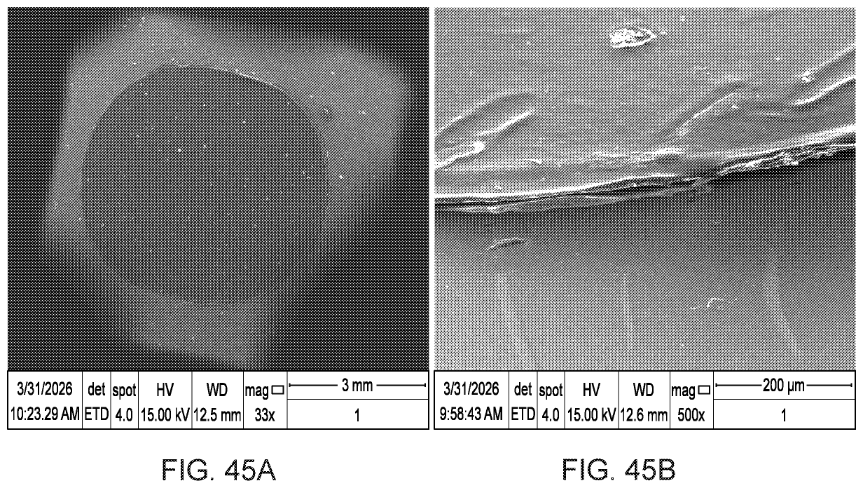

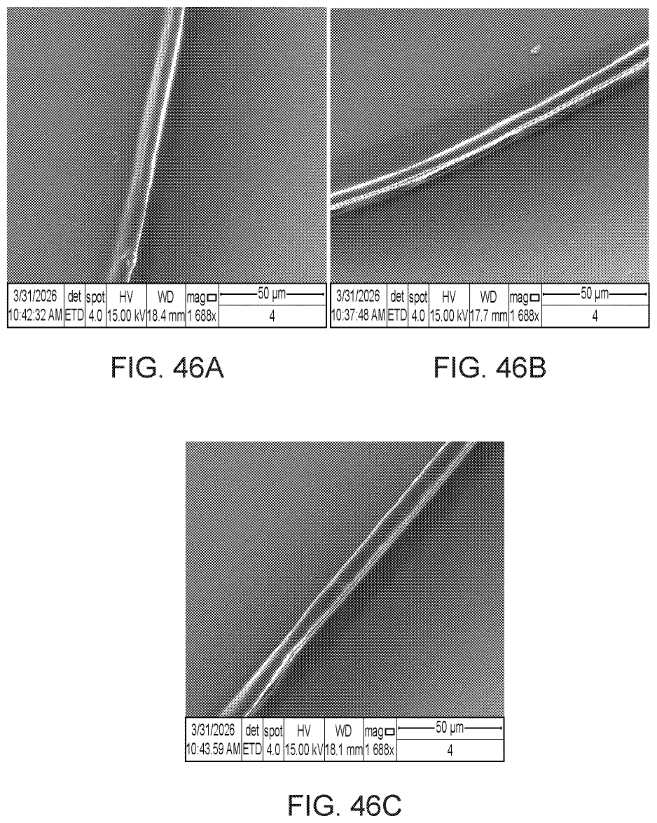

Referring to FIGS. 45A-45B, SEM images of capsulotomy edges created using the devices disclosed herein are shown, showing edge morphology at high magnification for assessment of smoothness, uniformity, and absence of gross irregularity, serrations, or partial tears. Referring to FIGS. 46A-46C, comparator SEM images of capsulotomy edges created by manual CCC are shown. The manual CCC SEM images are included as a comparator to allow direct qualitative comparison of edge morphology between manual tearing and the Atlas OCC. The SEM comparisons are consistent with a smoother, more uniform edge produced by the disclosed devices relative to manual CCC.

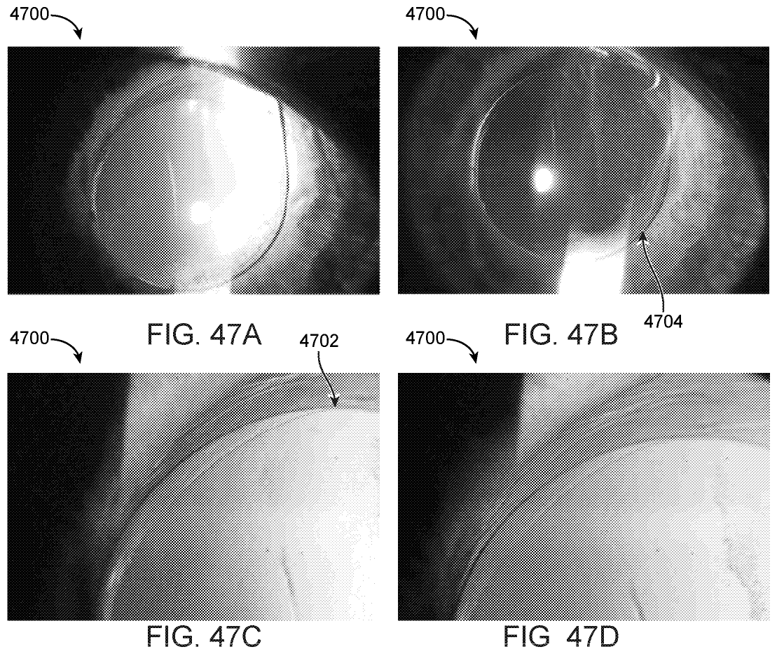

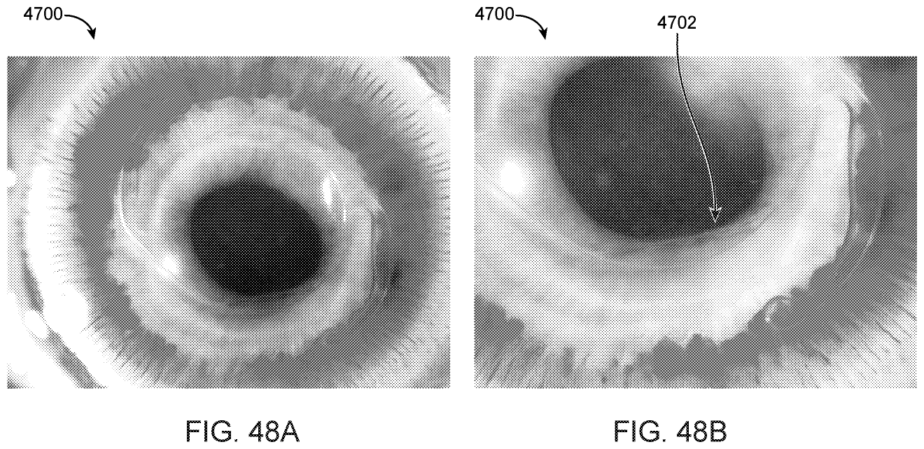

Referring to FIGS. 47A-47D, postoperative week-2 slit-lamp images from a rabbit eye 4700 following capsulotomy with the devices disclosed herein are shown, included as an early in vivo healing and positioning assessment after the procedure. The images show a stable rhexis edge 4702 and optic capture 4704, indicating stability of the capsule after the Atlas OCC procedure. Referring to FIGS. 48A-48B, higher-magnification postoperative slit-lamp views focused on the rhexis edge 4702 of a postoperative rabbit model 4700 are shown, highlighting edge integrity and appearance following healing. The higher-magnification views of the rhexis edge 4702 further support integrity and stability of the capsule after the Atlas OCC procedure over a short-term in vivo follow-up period.

Together, the biomechanical, thermal safety, edge morphology, and in vivo healing data described above support that the devices, systems, and methods disclosed herein can produce a capsulotomy (i) that may be at least as strong as, and in some respects stronger than, a manually created CCC; (ii) that may be created with limited collateral thermal exposure to the anterior chamber; (iii) that can exhibit a clean and uniform edge comparable to or smoother than a manual CCC edge; and (iv) that can maintain capsular stability over an early postoperative healing period. The Atlas OCC method with the one-click capsulotomy devices disclosed herein can produce capsulotomy edges that may be significantly stronger in peak tear force and may exhibit significantly greater displacement at peak force than the manual method. These performance attributes are consistent with, and can be attributable to, the geometry-driven, low thermal mass, pulsed ablation approach used in combination with the gimballed contact frame and the discrete-state delivery devices described herein. The combination of the geometry-driven cutting element, the gimballed contact frame for uniform capsular contact, and the delivery devices that provide controlled deployment and retraction of the cutting element may together contribute to the reproducibility and edge quality observed in the testing results.

### Junction Geometry and Current Flow Management

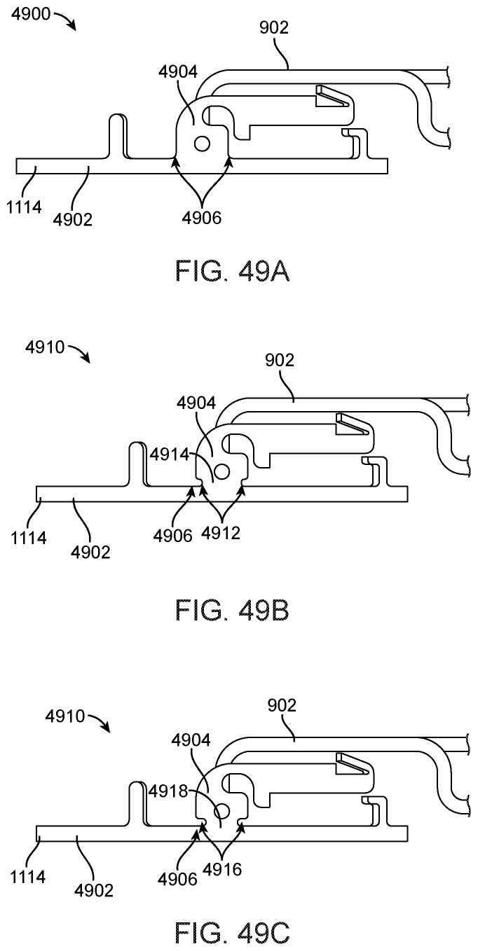

Referring generally to FIGS. 49A-49C, a conductive heating element ring assembly is shown, such as an example heating element ring 4900, 4910, having a substantially continuous ring body 4902 configured to deliver thermal energy along a closed perimeter. In some embodiments, the heating element 1114 (e.g., conductive loop, contact frame, ring), also referred to as a cutting element, can be electrically coupled to one or more conductive attachment features, such as a weld tab 4904, at a corresponding junction region 4906, and to one or more wires 902 (e.g., lead wires or conductive wires). In some embodiments, the heating element ring assembly 4910 can further comprise one or more notches 4912, 4916 disposed at or proximate to the junction region 4906 between the ring body 4902 and one or more weld tabs 4904. The one or more notches 4912, 4916 can be configured to locally increase the electrical resistance of the heating element 1114 at the junction region 4906, thereby directing additional thermal energy to the junction region 4906 during a current pulse and reducing or eliminating localized cold spots that might otherwise result in incomplete tissue severance (e.g., residual tissue tags) along the perimeter of the capsulotomy.

The one or more notches 4912, 4916 can comprise material removed from the heating element 1114 or weld tab 4904 at or adjacent to the junction region 4906. The notches 4912, 4916 can be formed by laser cutting, mechanical cutting, chemical etching, or other material removal processes. In some cases, the one or more notches 4912, 4916 can be disposed on opposing sides of the weld tab 4904 (e.g., at approximately the three o'clock and nine o'clock positions of the circular heating element 1114). Formation of the notches 4912, 4916 can locally narrow the weld tab 4904 of the heating element 1114 at the junction region 4906, thereby defining a reduced cross-sectional area 4914, 4918 and increasing the local electrical resistance relative to the remainder of the ring body 4902 perimeter.

The geometry of the one or more notches 4912, 4916 (e.g., depth, width, shape, number) can be selected to balance increased localized energy delivery at the junction region 4906 against structural integrity of the heating element assembly 4910 at the notch location. In some embodiments, a relatively smaller (more conservative) notch 4912 can define a reduced cross-sectional area 4914 that can provide a sufficient localized electrical resistance increase while maintaining structural robustness of the ring body 4902 to avoid fracture of the heating element 1114 during deployment, use, or retraction. In other embodiments, a relatively larger notch 4916 can define a further reduced cross-sectional area 4918, providing a greater localized resistance increase and thermal energy delivery at the junction region 4906, while increasing the susceptibility to mechanical stress at the notch 4916. The notch geometry can be selected based on factors including the material (e.g., nitinol) of the heating element 1114, the cross-sectional dimensions of the ring body 4902, and a target pulse energy.

FIGS. 49A-49C illustrate side views of example heating element ring assemblies 4900, 4910 in accordance with example embodiments described herein. FIG. 49A shows a heating element ring assembly 4900 in which the ring body 4902 is coupled to a weld tab 4904 at a junction region 4906 without notches, such that the junction region 4906 lacks geometric modification for resistance tuning and may be subject to localized thermal sinking during a current pulse. FIGS. 49B and 49C show the example heating element ring assembly 4910 comprising one or more notches 4912, 4916 formed at or proximate to the junction region 4906. In FIG. 49B, the heating element ring assembly 4910 comprises one or more relatively smaller junction notches 4912 defining the reduced cross-sectional areas 4914, while in FIG. 49C, the heating element ring assembly 4910 comprises one or more relatively larger notches 4916 defining further reduced cross-sectional areas 4918.

In some cases, the notches 4912, 4916 can be disposed at positions along the heating element ring assembly 4910 where the heating element 1114 is electrically coupled to one or more conducting wires 902, such as at solder joints, laser welds, or other electrical connections with the weld tab 4904. The notches 4912, 4916 can compensate for thermal sinking effects at the junction region 4906, where the attached wires 902 or weld tabs 4904 might otherwise conduct heat away from the ring body 4902 and reduce the local temperature below an ablation threshold. By increasing local electrical resistance at the junction region 4906, the heating element 1114 can be configured to achieve substantially uniform ablation temperatures around the ring perimeter during a single pulse or series of pulses.

### Electrical Systems

Provided herein are electrical systems disposed in the handle and distal end for activating the cutting elements described herein using the methods described herein.

The devices, systems, and methods described herein can be configured to make a complete circular cut in the capsule of the eye using heat from a cutting element. The function of the electronics can be to deliver a precise amount of energy to the cutting element in order to raise a temperature of the cutting element quickly under the control of the user. The systems can use a small battery (e.g., a lithium polymer (Li-Po) battery) to power the device. The small size and weight can be tailored for the user to have good tactile feedback as the cutting element is placed against the eye of the patient. Because a small battery may have limited current capability, the small battery may not have the ability to drive the cutting element directly. For this reason, a capacitor bank can be charged up slowly by the battery. The energy can then be delivered into the cutting element in a short burst (e.g., less than about 0.25 seconds) to make the cut.

### Capacitor Bank

While capacitors can be relatively lightweight compared to batteries, the physical size can get large for large capacitances required. The amount of energy stored in the capacitor bank can be governed by the equation for electric potential energy. Higher voltages may be used to minimize the physical size of the capacitor bank. Although capacitors get larger when increasing the rated voltage of the capacitor, the size increase can be outweighed by the large reduction in capacitance.

In some cases, the capacitor bank disclosed herein can use capacitors sufficient to accommodate the controlled voltage energy pulses disclosed above. As described above, the controlled voltages can be from about 1 V to about 240 V. The controlled voltages can be from about 1 V to about 20 V. The controlled voltages can be from about 1 V to about 10 V. If the full, or substantially the full (e.g., to maintain a safety margin) voltage from the capacitor bank is discharged directly into a resistive load such as the cutting element, the initial instantaneous current can be large, as governed by Ohm's law.

By adding a DC-DC buck converter that reduces voltage after the capacitor bank, the system can provide a constant voltage, which may result in constant current through the cutting element. The constant voltage output can greatly reduce the current requirements from the capacitor, allowing the use of a generic aluminum electrolytic capacitor rather than capacitors with high ripple-current capability and low ESR (effective series resistance). These generic aluminum electrolytic capacitors may be less expensive, more available, and also have a smaller physical size for a given capacitance. However, low ESR can still be important in selecting a capacitor, since some of the energy may be wasted in the form of heat.

### DC-DC Boost Converter

A small battery may have a nominal voltage of between about 3 V to about 4 V. In order to allow a single battery to charge the capacitor bank with a higher voltage, a DC-DC boost converter can be used. There are multiple characteristics that can be beneficial in a boost converter.

Constant current output can be important, because constant current output can allow for rapid and linear charging of the capacitor bank. The voltage of a capacitor when charged with constant current and the voltage on the capacitor bank can increase linearly with time. By contrast, using a constant voltage DC-DC boost converter may be accompanied by a current limiting resistor, which can result in an asymptotic approach to full charge, taking much longer.

The constant current can be controlled using pulse-width modulation (PWM) by a microcontroller unit (MCU).

In some cases, the DC-DC converter can disconnect an output of the DC-DC converter, such that when the capacitor bank is being discharged into the cutting element, the battery can be protected from excessive current draw.

Synchronous boost conversion can increase efficiency by replacing the low-side diode with an actively controlled MOSFET (metal-oxide-semiconductor field-effect transistor) synchronized to turn on and off in complement with a high-side MOSFET. The efficiency increase can allow the use of a smaller battery to deliver the same cumulative amount of energy into the cutting element before needing to be charged. In some cases, internal switching MOSFETs can reduce the size of the circuitry by having the switching MOSFETs inside the integrated circuit.

### DC-DC Buck Converter

The capacitor bank can be followed by a DC-DC buck converter. The output voltage from the DC-DC buck converter can determine how much current flows into the resistive cutting element and how quickly the energy from the capacitor bank is delivered. Multiple characteristics can be beneficial in a buck converter.

Synchronous buck conversion can increase efficiency by replacing the low-side diode with an actively controlled MOSFET that is synchronized to turn on and off in complement with the high-side MOSFET.

In some cases, a low minimum VIN can allow the system to draw as much energy from the capacitor bank as possible by continuing to operate until the capacitor bank voltage is low (e.g., almost fully depleted). The target voltage can be VCBmin (capacitor bank voltage minimum).

In some cases, a high current output can be provided to discharge the capacitor bank quickly and make the cut in the capsule, so high current capability can be important.

A high maximum V_{IN} can handle the high VCB (capacitor bank voltage) when the capacitor bank is fully charged. This VCB voltage can change with the measured material resistance (e.g., nitinol resistance), so the converter may be able to handle the highest VCB used in the device.

Enable control can be used to turn the output of the buck converter on/off, thereby allowing for a removal of a MOSFET switch to deliver the current to the cutting element. Enable control can also allow selection of the high current path or the low current path for measuring the resistance of the cutting element.

### Resistance Measurement of Cutting Element

To deliver consistent performance and minimize variable resistance in the cutting element, the resistance of each cutting element can be measured before calculating the amount of energy to be delivered. Losses can exist in the delivery path of the energy from the capacitor bank into the cutting element. Measuring the resistance of the cutting element can allow for calculating how much of the energy from the capacitor bank can be delivered to the cutting element. The capacitor bank voltage can then be adjusted to deliver the proper amount of energy to the cutting element.

The measured resistance can be a test criterion to make sure the cutting element is non-defective before a surgeon uses the cutting element on a patient. For example, measuring the resistance can determine whether a wire is broken, whether a laser weld between copper wire and nitinol is faulty, whether one half of the ring is severed, etc.

The resistance can be used to determine which variation (diameter, thickness, etc.) of the cutting element is being used.

In order to measure the resistance of the cutting element, which may be significantly less than 1 ohm, a precision current source can be used to measure accurately without overheating and damaging the cutting element. The precision current source can put a known current through the cutting element and read the voltage across the cutting element using a differential voltage amplifier. In some cases, two paths to the cutting element may exist (e.g., through the converters or the precision current source). In some cases, only one of the two paths to the cutting element can be enabled at any one time.

### Peripheral Components

Besides the circuitry described above having to do with the cutting element, additional components can be included. The components can comprise one or more of: an on/off modulator connecting the battery to the rest of the circuitry; a microcontroller linked to a user interface which makes measurements and calculations and controls the overall functionality of the device; a 3.3V Buck Regulator to power the low voltage sections of the device; a display to provide the user interface for the surgeon and used to show the firmware version, battery status, resistance measurement of the cutting element, or used with the Up and Down buttons to adjust the energy delivered; a Trigger button (button 704 described above) that can be used by the surgeon to deliver the energy to the cutting element to execute the cut; Up and Down buttons that can optionally be used with the Display to adjust the amount of energy delivered; a Piezo Buzzer to alert the surgeon of the impending energy delivery; and a Charging Circuit for the battery, which can be user replaceable or charged in-situ with an external charger.

In some cases, the display can be optional. An RGB LED can be another optional component that can provide user feedback in addition to, or instead of, the display.

### Delayed Energy Delivery

In some cases, a user may press the Trigger button accidentally. When an accidental press occurs, the cutting element can be damaged. For this reason, the system can have a delayed energy delivery scheme, where the Trigger button can be held down for a full second before the energy is delivered to the cutting element to make the cut. During the one-second delay, the user can be warned through the use of rhythmic beeps from the piezo buzzer. As a non-limiting example, three short beeps can occur at 250 ms intervals with a fourth, long beep at the one-second mark, at which point the energy can be delivered to the cutting element.

### DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

“Heating element”, “heated element”, “contact frame”, “frame”, “loop”, “ring”, “cutting element”, and other similar terminology may be used herein to refer to the heating element for forming a hole in a capsular bag.

The ability of the heating element to be folded into a configuration for insertion into the eye can also include being folded into the same or a different configuration for removal from the eye.

Although direct current (DC) is described in many of the scenarios, delivery devices, cutting elements, and handpieces above, all of the devices, systems, and methods disclosed herein can use AC power as an alternative to, or in addition to (e.g., dual powering modes), DC current. Both DC and AC power can be used with regular and ultrashort pulses.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers, and also represent an amount close to the stated amount that still performs a desired function or achieves a desired result. The term “about” or “approximately” may mean within an acceptable error range for the particular value, which will depend in part on how the value is measured or determined, e.g., the limitations of the measurement system. For example, the terms “approximately”, “about”, and “substantially” may refer to an amount that is within less than 10% of, within less than 5% of, within less than 1% of, within less than 0.1% of, and within less than 0.01% of the stated amount. For example, “about” may mean within 1 or more than 1 standard deviation, per the practice in the art. Alternatively, “about” may mean a range of up to 20%, up to 10%, up to 5%, or up to 1% of a given value. As used herein, the term “about” a number refers to that number plus or minus 10% of that number. The term “about” a range refers to that range minus 10% of its lowest value and plus 10% of its greatest value. Where particular values are described in the application and claims, unless otherwise stated the term “about” meaning within an acceptable error range for the particular value may be assumed.

As used in the specification and claims, the singular forms “a”, “an” and “the” include plural references unless the context clearly dictates otherwise. For example, the term “a sample” includes a plurality of samples, including mixtures thereof.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, the term “substantially” in reference to a given parameter, property, or condition means and includes to a degree that one of ordinary skill in the art would understand that the given parameter, property, or condition is met with a degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The subject can be a mammal. The mammal can be a human. The subject may be diagnosed or suspected of being at high risk for a disease. In some cases, the subject is not necessarily diagnosed or suspected of being at high risk for the disease.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### EXAMPLES

The following examples are included for illustrative purposes only and are not intended to limit the scope of the present disclosure. Unless otherwise stated, specific dimensions, materials, voltage levels, pulse durations, and procedural details are provided by way of example.

### Example 1: Standard 3 mm Capsulotomy in Adult Cataract Surgery

A handheld probe containing a 3 mm circular nitinol loop (50 μm wire) is introduced through a 2.2 mm clear corneal incision. The loop is deployed inside the anterior chamber and placed flush against the anterior capsule. A single 12 V, 30 ms direct-current pulse is delivered. The anterior capsule is ablated circumferentially, creating a smooth, free-floating 3 mm circular disc. The entire cut completes in less than 0.05 seconds. No collateral thermal damage was observed on the posterior capsule, iris, or corneal endothelium in this example.

### Example 2: Pediatric Cataract, 4 mm Loop

A 4 mm loop made of platinum-iridium alloy (40 μm wire) is inserted through a 2.4 mm incision. Pediatric capsules are highly elastic and prone to radial extension tears with manual CCC. The loop is deployed and energized with a 10 V, 20 ms pulse. This results in a substantially circular capsulotomy despite the high elasticity of the capsule. Intraoperative OCT confirms no thermal spread to surrounding tissues.

### Example 3: Intumescent Cataract, 5 mm Loop

In an intumescent cataract, intracapsular pressure creates a high risk of radial tear when attempting manual capsulorhexis. A 5 mm nitinol loop (70 μm wire) is folded into a tube for insertion through a 2.8 mm incision. Once deployed, the loop is positioned on the capsule and activated with a 15 V, 10 ms pulse. The anterior capsule opens instantaneously and uniformly, thereby reducing the risk of the “Argentinean flag sign” of uncontrolled radial tears.

### Example 4: Small Pupil Case, Elliptical Loop

In a case with a 3.5 mm pupil, a 3 mm×2 mm elliptical loop is delivered via a sheath and deployed beneath the iris. A 9 V, 12 ms pulse is applied. The resulting elliptical capsulotomy allows adequate access for lens removal in this case and maintains centration within the visual axis.

### Example 5: Posterior Capsulotomy

A 2 mm loop made of tungsten wire (30 μm) is inserted through a pars plana microincision. Once positioned against the posterior capsule, a 7 V, 5 ms DC pulse is delivered. A precise posterior opening is created, thereby eliminating or reducing the need for Nd: YAG (neodymium-doped yttrium aluminum garnet) laser postoperatively.

### Example 6: Capsular Relaxing Incisions

A 6 mm nitinol loop pre-formed with four evenly spaced radial protrusions is deployed inside the anterior chamber. In this example, each protrusion creates a small radial relaxing incision when energized with a 14 V, 6 ms pulse. This relieves capsular fibrosis in a pseudophakic eye with capsule contraction syndrome.

### Example 7: Iridotomy

A 1.5 mm circular loop of platinum-iridium (25 μm) is deployed through a 1.8 mm corneal incision and placed against the peripheral iris. A 10 V, 3 ms pulse is delivered, creating a clean iridotomy suitable for treating angle-closure glaucoma. No collateral iris pigment dispersion or corneal endothelial damage is observed.

### Example 8: Glaucoma Goniotomy

A 2 mm semi-circular loop is deployed via a gonioscopic approach into the trabecular meshwork. A 12 V, 10 ms pulse is applied, creating a precise opening in Schlemm's canal to enhance aqueous outflow.

### Example 9: Posterior Capsule Opacification Prevention (Primary Posterior Capsulotomy)

During pediatric cataract surgery, a 3 mm loop is positioned against the posterior capsule before IOL insertion. A 9 V, 6 ms pulse ablates a central posterior capsulotomy, preventing secondary visual axis opacification.

### Example 10: Foldable 7 mm Loop for Premium IOLs

A 7 mm nitinol loop is folded into a tubular shape, inserted through a 3 mm incision, and deployed inside the eye. Energized with a 20 V, 15 ms pulse, the device creates a large, well-centered capsulotomy for a multifocal IOL. Despite the large opening, no collateral damage occurs due to the ultralow thermal mass of the loop.

### Example 11: Multi-Loop Cartridge System

A handpiece contains a disposable cartridge with interchangeable 2 mm, 3 mm, and 5 mm loops. In a single surgical session, the surgeon performs a 5 mm anterior capsulotomy followed by a 2 mm posterior capsulotomy without changing the handpiece, simply swapping cartridges. Both cuts are completed with <15 V pulses of <10 ms duration.

### Example 12: Veterinary Cataract Surgery

A 4 mm loop of stainless-steel wire (80 μm) is inserted through a 3 mm incision into a canine eye with a cataract. A 15 V, 8 ms pulse creates a reproducible capsulotomy. The veterinary surgeon reports the method reduces surgical time and minimizes complications compared to manual CCC.

### Example 13: Emergency Field Kit Use

In a rural outreach camp, a battery-powered handheld probe with a 3.5 mm loop is used. Powered by rechargeable lithium-ion cells delivering 12 V, the loop completes a capsulotomy in 0.04 seconds. No external power supply, suction docking, or alignment system is required, enabling cataract surgery outside of traditional hospital settings.

### Example 14: Robotic-Assisted Cataract Surgery

A robotic cataract system incorporates a DC ultrashort pulsed loop controlled remotely. A 4.5 mm loop is deployed and energized with a 10 V, 5 ms pulse under robotic precision, yielding a reproducible capsulotomy without requiring human manual dexterity.

### Example 15: Disposable Single-Use Device

A single-use handpiece with integrated 3 mm loop and sealed lithium battery is activated by pressing a button. A single 12 V, 8 ms pulse completes the capsulotomy. The entire device is discarded after use, eliminating sterilization requirements.

### Example 16: Adult Capsulotomy With Manual Handpiece

A tip with a 5 mm circular frame and a thin nitinol closed-loop conductor is attached to a manual handpiece. The controller delivers a single 5 V, 20 A, 90 ms pulse in one implementation. The capsulotomy is completed in less than 0.1 s of energy application, with uniform cut quality around the perimeter.

### Example 17: Pediatric Capsulotomy

A 3.0 mm frame tip is used. The portable controller is set to a lower energy profile. The gimballed frame ensures uniform contact despite the highly elastic capsule. A 10 V, 50 ms pulse completes the opening.

### Example 18: Robotic Use

A robotic arm holds an adapter with a 6.0 mm frame tip. After the robot deploys the frame and gently contacts the capsule, a supervisory computer commands the controller to deliver a 15 V, 80 ms pulse. The gimbal compensates for slight tilt, and the capsulotomy is reproducible across multiple cases with minimal adjustment of robotic trajectories.

### Example 19: Portable Clinic Setting

The controller, powered by its internal battery, is used in an ambulatory center without large, fixed equipment. Multiple disposable tips are used sequentially; each capsulotomy requires only one brief pulse, and the battery is sufficient for many cases before recharging.

### Example 20: Tear Strength of Capsular Bag

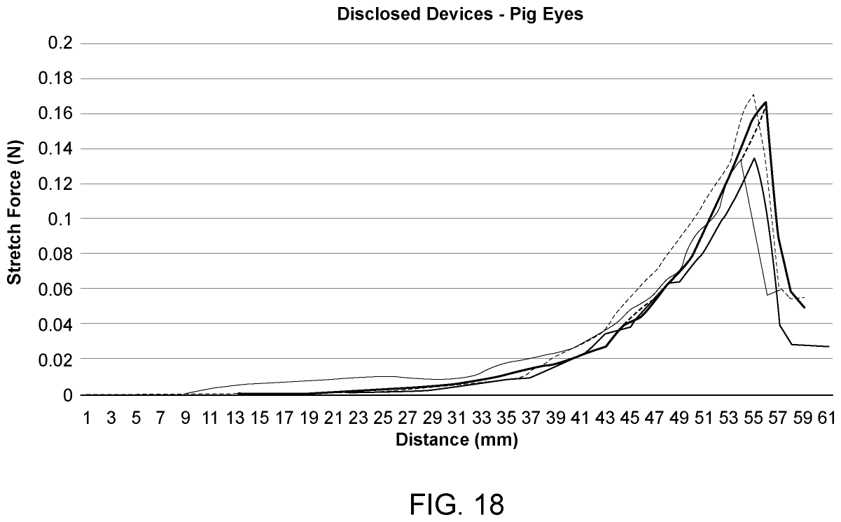

The devices disclosed herein were used in pig and rabbit eyes to confirm tear strength of the capsular bag. FIG. 18 shows a graph of the use of the devices disclosed herein in pig eyes. The Y-axis shows measured values for the tear strength of the capsular bag using the methods and devices disclosed herein (the three darker lines) compared to manual capsulorhexis (the lightest line). The X-axis is a relative scale of distance. The methods and devices disclosed herein did not measurably compromise the bag strength and actually had a higher peak tear force, which is beneficial. The pulses for the darker lines (methods and devices disclosed herein) were at 4 V to 5 V with 15 Amp to 20 Amp and a pulse width from about 90 ms to about 105 ms.

### Example 21: Device Operation

One non-limiting example of the operation of the device can be summarized as follows:
1. Li-Po Battery is charged in preparation for use.
2. A new Tip (e.g., distal end) with cutting element is attached to the Handle.
3. On/Off Switch is turned on to switch power to the device.
4. The Display shows the firmware revision and the battery status.
5. Optionally, the surgeon may increase or decrease the amount of Energy to be delivered using the Up/Down buttons and the Display.
6. Resistance of the cutting element is measured by the following steps, and is done quickly as to not heat the cutting element excessively: MOSFET powering the Precision Current Source is turned on; a known current is delivered to the cutting element; differential voltage across the cutting element is measured; resistance is calculated using Ohm's law; and MOSFET is turned off to disable the Precision Current Source.
7. Target energy storage is calculated from the cutting element resistance, and a target Capacitor Bank voltage is calculated. Alternatively, if the cutting element resistance does not fall within the acceptable range, an error message is given to the surgeon via the Display, RGB LED and/or piezo buzzer, and the MCU does not proceed to the next step, requiring the unit to be turned off and Tip replaced.
8. The Capacitor Bank is charged and the charge maintained by: A. Enabling Constant-Current DC-DC Boost Converter to start charging the Capacitor Bank with PWM set to 100%. This is the rapid charging phase, and the Capacitor Bank voltage increases linearly. B. The MCU controls the PWM proportionally according to the error between the target voltage and actual voltage, so the PWM decreases as the Capacitor Bank approaches the target voltage. The Capacitor Bank has a finite leakage current, so the MCU continues to maintain the target voltage until the surgeon triggers the energy delivery to the cutting element.
9. The MCU waits for the surgeon to press and hold the Trigger button. Once the Trigger button is pressed, it is held for one full second to deliver the energy. During this period, the button is held down continuously. If the button is released anytime during this period, the MCU goes back to waiting for the Trigger button to be pressed. Also, if the MCU determines that the Capacitor Bank voltage is not being maintained (likely due to low battery), the Trigger button is disabled and an error message is given to the surgeon via the Display, RGB LED and/or piezo buzzer.

During Trigger button press, there are short beeps at 250 ms, 500 ms, and 750 ms from the piezo buzzer. There is a long beep at 1000 ms (1 second) simultaneous with delivery of energy to the cutting element. Once the Capacitor Bank voltage VCB gets down to VCBmin, the MCU turns off the DC-DC Buck converter.
10. Unit is turned off using the On/Off Switch and returns back to step 2 above.

One or more of the above steps may be omitted, reordered, or modified in other implementations.

### CLAUSES

1. A method of forming an opening in a capsular bag of an eye of a subject, the method comprising: changing a heating element into a tubular or elliptical configuration for insertion into the eye; and heating a portion of the capsular bag with the heating element to a target temperature such that tissue of the capsular bag is severed to form the opening.
2. The method of clause 1, further comprising placing the heating element proximate to or in contact with the portion of the capsular bag, wherein the portion of the capsular bag is heated via the heating element.
3. The method of clause 2, wherein the heating element is placed proximate the portion of the capsular bag.
4. The method of clause 2, wherein the heating element is placed in contact with the portion of the capsular bag.
5. The method of any one of clauses 2 to 4, wherein the heating element comprises a shape having a continuous boundary.
6. The method of any one of clauses 2 to 5, wherein the heating element comprises a distally-open recess or cavity disposed in the continuous boundary.
7. The method of any one of clauses 2 to 6, wherein the heating element comprises a ring shape.
8. The method of any one of clauses 2 to 7, wherein heating the portion of the capsular bag comprises applying a current to the heating element.
9. The method of clause 8, wherein the current comprises an alternating current.
10. The method of clause 8, wherein the current comprises a direct current.
11. The method of clause 10, wherein applying direct current to the heating element comprises applying at least one pulse of direct current.
12. The method of clause 10 or 11, further comprising detecting a temperature proximate the heating element and adjusting the application of direct current to the heating element based on the detected temperature.
13. The method of any one of clauses 10 to 12, wherein applying direct current to the heating element to heat the heating element comprises heating the heating element with resistive heating.
14. The method of any one of clauses 8 to 13, applying current to the heating element comprises applying current from a power source of the thermal device to the heating element.
15. The method of clause 14, wherein applying current comprises delivering, via the power source, pulses with a voltage from about 1 V to about 20 V.
16. The method of clause 15, wherein the pulses have a voltage from about 4 V to about 5 V.
17. The method of any one of clauses 1 to 16, further comprising removing the severed tissue.
18. The method of any one of clauses 1 to 17, further comprising removing a natural lens disposed within the capsular bag through the opening.
19. The method of any one of clauses 1 to 18, further comprising implanting an intraocular lens within the capsular bag through the opening.
20. The method of any one of clauses 1 to 19, wherein the target temperature is from about 60 °C to about 150 °C.
21. The method of any one of clauses 1 to 20, further comprising selecting a distal tip with the heating element from a plurality of distal tips having heating elements of different shapes and sizes based on a desired shape and size for the opening.
22. The method of any one of clauses 1 to 21, wherein no mechanical cutting is performed.
23. The method of any one of clauses 1 to 22, wherein a distal edge of the heating element is blunt.
24. The method of any one of clauses 1 to 23, further comprising moving the heating element along a path on an anterior surface of the capsular bag to form the opening.
25. The method of clause 24, further comprising applying a biocompatible dye on the anterior surface of the capsular bag to outline the path.
26. The method of clause 24 or 25, further comprising moving the heating element at a controlled rate along the path to regulate a total amount of heat energy conducted into a portion of capsular tissue.
27. The method of any one of clauses 1 to 26, wherein the heating element is conductive.
28. The method of any one of clauses 1 to 27, further comprising inserting the heating element through a corneal incision.
29. The method of any one of clauses 1 to 28, further comprising applying at least one ultrashort direct-current pulse to the heating element for a duration from about 10 microseconds to about 1 second, thereby ablating the capsular bag.
30. The method of any one of clauses 1 to 29, further comprising removing the heating element from the eye after formation of the opening.
31. The method of any one of clauses 1 to 30, wherein forming the opening in the capsular bag comprises an anterior capsulotomy for cataract surgery.
32. The method of any one of clauses 1 to 30, wherein forming the opening in the capsular bag comprises a posterior capsulotomy performed intraoperatively.
33. The method of any one of clauses 1 to 30, wherein the heating element creates a capsular relaxing incision for capsular fibrosis.
34. The method of any one of clauses 1 to 30, wherein the heating element is applied to an iris of the eye to create an iridotomy or iridectomy.
35. The method of any one of clauses 1 to 30, wherein the heating element is applied to the trabecular meshwork to create a glaucoma outflow channel.
36. The method of any one of clauses 1 to 30, wherein the heating element is applied in pediatric cataract cases with elastic capsules.
37. The method of any one of clauses 1 to 30, wherein the heating element is applied in intumescent cataract cases to prevent radial tears.
38. The method of any one of clauses 1 to 37, further comprising attaching a tip comprising the heating element, a closed-loop conductor, and a gimballed joint structure to a delivery device.
39. The method of clause 38, wherein the delivery device comprises a handpiece or a robotic adapter.
40. The method of any one of clauses 1 to 39, further comprising deploying the heating element to a deployment configuration having a predetermined perimeter.
41. The method of clause 40, wherein deploying the heating element comprises mechanically deploying the heating element.
42. The method of clause 40, wherein deploying the heating element comprises automatically deploying the heating element.
43. The method of any one of clauses 1 to 42, further comprising advancing the heating element until it contacts substantially an entire perimeter of an anterior lens capsule of the eye;
44. The method of clause 43, further comprising tilting the heating element via a gimballed joint structure to conform the contact frame to a surface of the anterior lens capsule.
45. The method of clause 43 or 44, further comprising actuating a portable controller to deliver at least one ultrashort direct-current pulse through a closed-loop conductor to thermally ablate the anterior lens capsule along the entire perimeter.
46. The method of clause 45, wherein the portable controller is battery powered and operates without connection to a large external console.
47. The method of clause 45 or 46, wherein the portable controller comprises a handpiece.
48. The method of any one of clauses 1 to 47, further comprising deploying the heating element to its shape-memory geometry.
49. The method of any one of clauses 1 to 48, further comprising extending the heating element into an anterior chamber of an eye upon deployment.
50. The method of clause 49, further comprising preventing activation of the heating element unless the heating element is confirmed to be deployed in the anterior chamber.
51. The method of any one of clauses 1 to 50, further comprising monitoring temperature of tissues adjacent to the capsular bag via a thermal sensor positioned near the heating element.
52. The method of any one of clauses 1 to 51, further comprising insulating the heating element to prevent thermal spread to an iris or corneal endothelium of the eye.
53. The method of any one of clauses 1 to 52, wherein the method is applied in human ophthalmic surgery.
54. The method of any one of clauses 1 to 52, wherein the method is applied in veterinary ophthalmic surgery.
55. The method of any one of clauses 1 to 52, wherein the method is applied in non-ophthalmic procedures comprising one or more of tympanostomy, dura opening, dermatologic excision, or endoscopic mucosal resection.
56. The method of any one of clauses 1 to 55, wherein the portion of the capsular bag comprises an anterior portion of the capsular bag.
57. The method of any one of clauses 1 to 56, wherein changing the heating element into a tubular or elliptical configuration comprises one or more of folding, compressing, or stretching the heating element.
58. A thermal device for forming an opening in a capsular bag of an eye of a subject, the thermal device comprising: a distal portion comprising a heating element configured to be changeable into a tubular or elliptical configuration for insertion into the eye; a power source configured to deliver energy to the heating element to heat the heating element to a target temperature such that when the heating element is positioned proximate to or in contact with tissue of the capsular bag, the tissue is severed to form the opening.
59. The thermal device of clause 58, wherein the power source is a battery.
60. The thermal device of clause 59, wherein the battery is rechargeable.
61. The thermal device of any one of clauses 58 to 60, further comprising a handle.
62. The thermal device of clause 61, wherein the distal tip is configured to be removably coupled to the handle.
63. The thermal device of clause 61 or 62, wherein the handle comprises the power source.
64. The thermal device of any one of clauses 61 to 63, wherein the power source is disposed inside a housing of the handle.
65. The thermal device of any one of clauses 61 to 64, wherein the distal portion is disposable and the handle is reusable.
66. The thermal device of any one of clauses 61 to 65, wherein the handle comprises a portable controller.
67. The thermal device of any one of clauses 58 to 66, wherein the heating element comprises a shape having a continuous boundary.
68. The thermal device of clause 67, wherein the heating element comprises a distally-open recess or cavity disposed in the continuous boundary.
69. The thermal device of any one of clauses 58 to 68, wherein the heating element comprises a ring shape.
70. The thermal device of any one of clauses 58 to 69, wherein an anterior portion of the capsular bag is heated via the heating element.
71. The thermal device of clause 70, wherein heating the anterior portion of the capsular bag comprises applying a current to the heating element.
72. The thermal device of clause 71, wherein the current comprises an alternating current.
73. The thermal device of clause 71, wherein the current comprises a direct current.
74. The thermal device of clause 73, further comprising a temperature sensor to detect temperature, wherein the power source is configured to adjust the direct current applied to the heating element in response to the detected temperature.
75. The thermal device of clause 73 or 74, wherein the direct current applied to the heating element resistively heats the heating element.
76. The thermal device of any one of clauses 73 to 75, wherein the distal tip comprises one or more conductors electrically connected to the heating element, the one or more conductors configured to deliver the direct current to the heating element.
77. The thermal device of clause 76, wherein the one or more conductors are configured to deliver direct current to the heating element and direct current away from the heating element.
78. The thermal device of clause 76 or 77, wherein the one or more conductors are disposed around the heating element.
79. The thermal device of any one of clauses 76 to 78, wherein the one or more conductors are looped around the heating element.
80. The thermal device of any one of clauses 76 to 78, wherein the one or more conductors are disposed within an interior of the heating element.
81. The thermal device of any one of clauses 58 to 80, wherein the heating element is placed proximate the anterior portion of the capsular bag.
82. The thermal device of any one of clauses 58 to 81, wherein the heating element is placed in contact against the anterior portion of the capsular bag.
83. The thermal device of any one of clauses 58 to 82, wherein the target temperature is from about 60 °C to about 150 °C.
84. The thermal device of any one of clauses 58 to 83, wherein the heating element comprises a shape and size that corresponds to a target shape and size for the opening.
85. The thermal device of any one of clauses 58 to 84, wherein the thermal device is devoid of a cutting edge configured to mechanically sever the tissue of the capsular bag.
86. The thermal device of any one of clauses 58 to 85, wherein a distal edge of the heating element is blunt.
87. The thermal device of any one of clauses 58 to 85, wherein a distal edge of the heating element is sharp.
88. The thermal device of any one of clauses 58 to 87, further comprising an insulated portion that holds the heating element.
89. The thermal device of clause 88, wherein a proximal portion of the heating element is disposed in the insulated portion and a distal portion of the heating element protrudes from the insulated portion.
90. The thermal device of clause 88, wherein the heating element is configured to be retracted into and deployed from the insulated portion.
91. The thermal device of any one of clauses 58 to 90, wherein the heating element comprises one or more of a metallic material, an alloy material, or nitinol.
92. The thermal device of any one of clauses 58 to 90, wherein the heating element comprises platinum, iridium, tungsten, stainless steel, cobalt-chrome, or combinations thereof.
93. The thermal device of any one of clauses 58 to 90, wherein the heating element comprises a composite material comprising one or more of carbon nanotube bundles, conductive polymers, or nanostructured alloys.
94. The thermal device of any one of clauses 58 to 93, wherein the heating element has a cross-section, wherein the cross-section is round, a flat ribbon, braided, or hollow-core.
95. The thermal device of any one of clauses 58 to 94, wherein the heating element is coated with an insulating or hydrophobic material.
96. The thermal device of any one of clauses 58 to 95, wherein a cutting surface of the heating element is not coated with an insulating or hydrophobic material.
97. The thermal device of any one of clauses 58 to 96, wherein the heating element forms a circular geometry between 1 mm and 7 mm in diameter.
98. The thermal device of any one of clauses 58 to 97, wherein the heating element forms an elliptical, polygonal, or lobed geometry.
99. The thermal device of any one of clauses 58 to 98, wherein the heating element comprises multiple concentric or overlapping heating elements.
100. The thermal device of any one of clauses 58 to 99, wherein the heating element is foldable into a tubular or elliptical configuration for insertion through an incision less than or equal to about 3 mm.
101. The thermal device of any one of clauses 58 to 100, wherein the heating element is configured to be deployed to its shape-memory geometry.
102. The thermal device of any one of clauses 58 to 101, wherein the heating element is housed within a retractable sheath and configured to be extended into an anterior chamber of an eye upon deployment.
103. The thermal device of clause 102, wherein activation of the heating element is prevented unless the heating element is confirmed to be deployed in the anterior chamber.
104. The thermal device of any one of clauses 58 to 103, further comprising a thermal sensor positioned near the heating element to monitor temperature.
105. The thermal device of any one of clauses 58 to 104, wherein the heating element is insulated to prevent thermal spread to an iris or corneal endothelium of the eye.
106. The thermal device of any one of clauses 58 to 105, wherein the heating element is configured to be applied in human ophthalmic surgery.
107. The thermal device of any one of clauses 58 to 105, wherein the heating element is configured to be applied in veterinary ophthalmic surgery.
108. The thermal device of any one of clauses 58 to 105, wherein the heating element is configured to be applied in non-ophthalmic procedures comprising one or more of tympanostomy, dura opening, dermatologic excision, or endoscopic mucosal resection.
109. The thermal device of any one of clauses 58 to 108, wherein the heating element is configured to be inserted into the eye through a corneal incision.
110. The thermal device of any one of clauses 58 to 109, wherein the heating element comprises a low thermal mass conductive loop.
111. The thermal device of any one of clauses 58 to 110, wherein the power source is configured to deliver pulses with a voltage from about 1 V to about 20 V.
112. The thermal device of clause 111, wherein the pulses have a voltage from about 4 V to about 5 V.
113. The thermal device of any one of clauses 58 to 112, wherein the heating element has a closed perimeter.
114. The thermal device of any one of clauses 58 to 113, wherein the heating element is configured to be changeable into a tubular or elliptical configuration via one or more of folding, compressing, or stretching the heating element.
115. The thermal device of any one of clauses 58 to 114, wherein the distal portion comprises one or more stabilization features in contact with the heating element.
116. The apparatus of clause 115, wherein the one or more stabilization features comprise a proximal stabilization feature and a distal stabilization feature.
117. The apparatus of clause 116, wherein the proximal stabilization feature is configured to prevent catching of a proximal end of the heating element on the eye after opening the capsular bag.
118. A thermal device for forming an opening in a capsular bag of an eye of a subject, the thermal device comprising: a distal portion comprising a heating element; a power source configured to deliver pulses of energy with a voltage from about 1 V to about 20 V to the heating element to heat the heating element to a target temperature, such that when the heating element is positioned proximate to or in contact with tissue of the capsular bag, the tissue is severed to form the opening.
119. The thermal device of clause 118, wherein the power source is a battery.
120. The thermal device of clause 119, wherein the battery is rechargeable.
121. The thermal device of any one of clauses 118 to 120, further comprising a DC-DC boost converter configured to boost an output of the power source.
122. The thermal device of any one of clauses 118 to 121, further comprising a handle.
123. The thermal device of clause 122, wherein the distal tip is configured to be removably coupled to the handle.
124. The thermal device of clause 122 or 123, wherein the handle comprises the power source.
125. The thermal device of any one of clauses 122 to 124, wherein the power source is disposed inside a housing of the handle.
126. The thermal device of any one of clauses 122 to 125, wherein the distal portion is disposable and a handle is reusable.
127. The thermal device of clause 126, wherein the heating element comprises a distally-open recess or cavity disposed in a continuous boundary.
128. The thermal device of any one of clauses 118 to 127, wherein the heating element comprises a ring shape.
129. The thermal device of any one of clauses 118 to 128, wherein the anterior portion of the capsular bag is heated via the heating element.
130. The thermal device of any one of clauses 118 to 129, wherein the power source is configured to deliver pulses of alternating current.
131. The thermal device of any one of clauses 118 to 129, wherein the power source is configured to deliver pulses of direct current.
132. The thermal device of clause 131, further comprising a temperature sensor to detect temperature, wherein the power source is configured to adjust the direct current applied to the heating element in response to the detected temperature.
133. The thermal device of clause 131 or 132, wherein the direct current applied to the heating element resistively heats the heating element.
134. The thermal device of any one of clauses 131 to 133, wherein the distal tip comprises one or more conductors electrically connected to the heating element, the one or more conductors configured to deliver the direct current to the heating element.
135. The thermal device of clause 134, wherein the one or more conductors are disposed around the heating element.
136. The thermal device of clause 134 or 135, wherein the one or more conductors are looped around the heating element.
137. The thermal device of clause 134 or 135, wherein the one or more conductors are disposed within an interior of the heating element.
138. The thermal device of any one of clauses 118 to 137, wherein the heating element is placed proximate an anterior portion of the capsular bag.
139. The thermal device of any one of clauses 118 to 138, wherein the heating element is placed in contact against an anterior portion of the capsular bag.
140. The thermal device of any one of clauses 118 to 139, wherein the target temperature is from about 60 °C to about 150 °C.
141. The thermal device of any one of clauses 118 to 140, wherein the heating element comprises a shape and size that corresponds to a target shape and size for the opening.
142. The thermal device of any one of clauses 118 to 141, wherein the thermal device is devoid of a cutting edge configured to mechanically sever the tissue of the capsular bag.
143. The thermal device of any one of clauses 118 to 142, wherein a distal edge of the heating element is blunt.
144. The thermal device of any one of clauses 118 to 142, wherein a distal edge of the heating element is sharp.
145. The thermal device of any one of clauses 118 to 144, further comprising an insulated portion that holds the heating element.
146. The thermal device of clause 145, wherein a proximal portion of the heating element is disposed in the insulated portion and a distal portion of the heating element protrudes from the insulated portion.
147. The thermal device of clause 146, wherein the heating element is configured to be retracted into and deployed from the insulated portion.
148. The thermal device of any one of clauses 118 to 147, wherein the heating element comprises one or more of a metallic material, an alloy material, or nitinol.
149. The thermal device of any one of clauses 118 to 147, wherein the heating element comprises platinum, iridium, tungsten, stainless steel, cobalt-chrome, or combinations thereof.
150. The thermal device of any one of clauses 118 to 147, wherein the heating element comprises a composite material comprising one or more of carbon nanotube bundles, conductive polymers, or nanostructured alloys.
151. The thermal device of any one of clauses 118 to 150, wherein the heating element has a cross-section, wherein the cross-section is round, a flat ribbon, braided, or hollow-core.
152. The thermal device of any one of clauses 118 to 151, wherein the heating element is coated with an insulating or hydrophobic material.
153. The thermal device of any one of clauses 118 to 152, wherein a cutting surface of the heating element is not coated with an insulating or hydrophobic material.
154. The thermal device of any one of clauses 118 to 153, wherein the heating element forms a circular geometry between 1 mm and 7 mm in diameter.
155. The thermal device of any one of clauses 118 to 154, wherein the heating element forms an elliptical, polygonal, or lobed geometry.
156. The thermal device of any one of clauses 118 to 155, wherein the heating element comprises multiple concentric or overlapping heating elements.
157. The thermal device of any one of clauses 118 to 156, wherein the heating element is configured to be deployed to its shape-memory geometry.
158. The thermal device of any one of clauses 118 to 157, wherein the heating element is housed within a retractable sheath and configured to be extended into an anterior chamber of an eye upon deployment.
159. The thermal device of clause 158, wherein activation of the heating element is prevented unless the heating element is confirmed to be deployed in the anterior chamber.
160. The thermal device of any one of clauses 118 to 159, further comprising a thermal sensor positioned near the heating element to monitor temperature.
161. The thermal device of any one of clauses 118 to 160, wherein the heating element is insulated to prevent thermal spread to an iris or corneal endothelium of the eye.
162. The thermal device of any one of clauses 118 to 161, wherein the heating element is configured to be applied in human ophthalmic surgery.
163. The thermal device of any one of clauses 118 to 161, wherein the heating element is configured to be applied in veterinary ophthalmic surgery.
164. The thermal device of any one of clauses 118 to 161, wherein the heating element is configured to be applied in non-ophthalmic procedures comprising one or more of tympanostomy, dura opening, dermatologic excision, or endoscopic mucosal resection.
165. The thermal device of any one of clauses 118 to 164, wherein the heating element is configured to be inserted into the eye through a corneal incision.
166. The thermal device of any one of clauses 118 to 165, wherein the heating element comprises a low thermal mass conductive loop.
167. The thermal device of any one of clauses 118 to 166, wherein the heating element has a closed perimeter.
168. The thermal device of any one of clauses 118 to 167, wherein the pulses of energy have a voltage from about 4 V to about 5 V.
169. The thermal device of any one of clauses 118 to 168, wherein the distal portion comprises one or more stabilization features in contact with the heating element.
170. The apparatus of clause 169, wherein the one or more stabilization features comprise a proximal stabilization feature and a distal stabilization feature.
171. The apparatus of clause 170, wherein the proximal stabilization feature is configured to prevent catching of a proximal end of the heating element on the eye after opening the capsular bag.
172. An apparatus for forming an opening in a capsular bag of an eye of a subject, the apparatus comprising: a frame, wherein the frame comprises: a first side comprising a contact surface configured to contact the capsular bag; a second side comprising a delivery interface configured to couple to a delivery device; a gimbal configured to tilt the frame to maintain substantially full contact with the capsular bag within a predetermined angle range relative to an angle of the delivery device when the delivery device is coupled to the delivery interface, wherein the gimbal comprises one or more of: one or more posts disposed on the delivery interface and extending away from the first side; one or more hinges disposed on the delivery interface and extending away from the first side, wherein the hinges are configured to provide an open area for interfacing with the delivery device; or one or more slots proximal the one or more posts and configured to provide a slot for a distal end of the delivery device.
173. The apparatus of clause 172, further comprising an anchor configured to anchor the delivery interface to the delivery device.
174. The apparatus of clause 172 or 173, wherein the delivery device comprises one or more stabilization features configured to control the tilt of the frame.
175. The apparatus of clause 174, wherein the one or more stabilization features comprise a proximal stabilization feature and a distal stabilization feature.
176. The apparatus of clause 175, wherein the proximal stabilization feature is configured to prevent catching of a proximal end of the frame on the eye after opening the capsular bag.
177. The apparatus of any one of clauses 172 to 176, wherein the one or more hinges are disposed proximal to the one or more posts.
178. The apparatus of any one of clauses 172 to 177, wherein the one or more hinges comprise a rounded elbow structure surrounding the open area.
179. The apparatus of any one of clauses 172 to 178, wherein the open area is substantially circular or elliptical.
180. The apparatus of any one of clauses 172 to 179, wherein the gimbal is configured to allow pivoting of the frame on the capsular bag.
181. The apparatus of any one of clauses 172 to 180, wherein the predetermined angle range is from about 5 degrees to about 90 degrees relative to an axis.
182. The apparatus of any one of clauses 172 to 181, wherein the one or more slots are formed by a space between the one or more posts and the one or more hinges.
183. The apparatus of any one of clauses 172 to 182, wherein the one or more slots are distal to the one or more hinges.
184. The apparatus of any one of clauses 172 to 183, wherein the one or more posts comprises a plurality of posts mirrored along a central axis of the frame.
185. The apparatus of any one of clauses 172 to 184, wherein the one or more hinges comprises a plurality of posts mirrored along a central axis of the frame.
186. The apparatus of any one of clauses 172 to 185, wherein the one or more slots comprises a plurality of posts mirrored along a central axis of the frame.
187. The apparatus of any one of clauses 172 to 186, wherein a base of each of the one or more hinges has a bore configured to receive a conducting wire.
188. The apparatus of any one of clauses 172 to 187, wherein the frame is configured to be changeable into a tubular or elliptical configuration for insertion into the eye.
189. The apparatus of any one of clauses 172 to 188, wherein the frame is configured to be changeable into a tubular or elliptical configuration via one or more of folding, compressing, or stretching the heating element.
190. The apparatus of any one of clauses 172 to 189, wherein the frame comprises a shape having a continuous boundary.
191. The apparatus of any one of clauses 172 to 190, wherein the frame comprises a ring shape.
192. The apparatus of any one of clauses 172 to 191, wherein the frame is configured to be heated via the conducting wires.
193. The apparatus of any one of clauses 172 to 192, wherein an anterior portion of the capsular bag is heated via the frame.
194. The apparatus of clause 193, wherein heating the anterior portion of the capsular bag comprises applying a current to the frame.
195. The apparatus of clause 194, wherein the current comprises an alternating current.
196. The apparatus of clause 195, wherein the current comprises a direct current.
197. The apparatus of any one of clauses 172 to 196, wherein the contact surface does not comprise a cutting edge configured to mechanically sever the tissue of the capsular bag.
198. The apparatus of any one of clauses 172 to 197, wherein the heating element comprises one or more of a metallic material, an alloy material, or nitinol.
199. The apparatus of any one of clauses 172 to 197, wherein the frame comprises platinum, iridium, tungsten, stainless steel, cobalt-chrome, or combinations thereof.
200. The apparatus of any one of clauses 172 to 197, wherein the frame comprises a composite material comprising one or more of carbon nanotube bundles, conductive polymers, or nanostructured alloys.
201. The apparatus of any one of clauses 172 to 200, wherein the frame has a cross-section, wherein the cross-section is round, a flat ribbon, braided, or hollow-core.
202. The apparatus of any one of clauses 172 to 201, wherein the frame is coated with an insulating or hydrophobic material.
203. The apparatus of any one of clauses 172 to 202, wherein the contact surface is not coated with an insulating or hydrophobic material.
204. The apparatus of any one of clauses 172 to 203, wherein the frame forms a circular geometry between 1 mm and 7 mm in diameter.
205. The apparatus of any one of clauses 172 to 204, wherein the frame forms an elliptical, polygonal, or lobed geometry.
206. The apparatus of any one of clauses 172 to 205, wherein the frame comprises a shape-memory geometry.
207. The apparatus of any one of clauses 172 to 206, wherein the frame comprises a low thermal mass conductive loop.
208. A method of forming an opening in a capsular bag of an eye of a subject, the method comprising: actuating a controller to deliver at least one current pulse with a voltage from about 1 V to about 20 V through a closed-loop conductor to a heating element; and heating, via the heating element, a portion of the capsular bag to a target temperature such that tissue of the capsular bag is severed to form the opening.
209. The method of clause 208, further comprising placing the heating element proximate to or in contact with the anterior portion of the capsular bag.
210. The method of clause 209, wherein the heating element is placed proximate the anterior portion of the capsular bag.
211. The method of clause 209, wherein the heating element is placed in contact against the anterior portion of the capsular bag.
212. The method of any one of clauses 208 to 211, wherein the heating element comprises a shape having a continuous boundary.
213. The method of any one of clauses 208 to 212, wherein the heating element comprises a distally-open recess or cavity disposed in the continuous boundary.
214. The method of any one of clauses 208 to 213, wherein the heating element comprises a ring shape.
215. The method of any one of clauses 208 to 214, wherein the at least one current pulse comprises at least one alternating current pulse.
216. The method of any one of clauses 208 to 214, wherein the at least one current pulse comprises at least one direct current pulse.
217. The method of clause 216, further comprising detecting a temperature proximate the heating element and adjusting the delivery of direct current to the heating element based on the detected temperature.
218. The method of clause 216 or 217, wherein applying direct current to the heating element to heat the heating element comprises heating the heating element with resistive heating.
219. The method of any one of clauses 208 to 218, further comprising removing the severed tissue.
220. The method of any one of clauses 208 to 219, further comprising removing a natural lens disposed within the capsular bag through the opening.
221. The method of any one of clauses 208 to 220, further comprising implanting an intraocular lens within the capsular bag through the opening.
222. The method of any one of clauses 208 to 221, wherein the target temperature is from about 60 °C to about 150 °C.
223. The method of any one of clauses 208 to 222, further comprising selecting a distal tip with the heating element from a plurality of distal tips having heating elements of different shapes and sizes based on a desired shape and size for the opening.
224. The method of any one of clauses 208 to 223, wherein no mechanical cutting is performed.
225. The method of any one of clauses 208 to 224, wherein a distal edge of the heating element is blunt.
226. The method of any one of clauses 208 to 225, further comprising moving the heating element along a path on an anterior surface of the capsular bag to form the opening.
227. The method of clause 226, further comprising applying a biocompatible dye on the anterior surface of the capsular bag to outline the path.
228. The method of clause 226 or 227, further comprising moving the heating element at a controlled rate along the path to regulate a total amount of heat energy conducted into a portion of capsular tissue.
229. The method of any one of clauses 208 to 228, wherein the heating element is conductive.
230. The method of any one of clauses 208 to 229, further comprising inserting the heating element through a corneal incision.
231. The method of any one of clauses 208 to 230, further comprising removing the heating element from the eye after formation of the opening.
232. The method of any one of clauses 208 to 231, wherein forming the opening in the capsular bag comprises an anterior capsulotomy for cataract surgery.
233. The method of any one of clauses 208 to 231, wherein forming the opening in the capsular bag comprises a posterior capsulotomy performed intraoperatively.
234. The method of any one of clauses 208 to 231, wherein the heating element creates a capsular relaxing incision for capsular fibrosis.
235. The method of any one of clauses 208 to 231, wherein the heating element is applied to an iris of the eye to create an iridotomy or iridectomy.
236. The method of any one of clauses 208 to 231, wherein the heating element is applied to the trabecular meshwork to create a glaucoma outflow channel.
237. The method of any one of clauses 208 to 231, wherein the heating element is applied in pediatric cataract cases with elastic capsules.
238. The method of any one of clauses 208 to 231, wherein the heating element is applied in intumescent cataract cases to prevent radial tears.
239. The method of any one of clauses 208 to 238, further comprising attaching a tip comprising the heating element, a closed-loop conductor, and a gimballed joint structure to a delivery device.
240. The method of clause 239, wherein the delivery device comprises a handpiece or a robotic adapter.
241. The method of any one of clauses 208 to 240, further comprising deploying the heating element to a deployment configuration having a predetermined perimeter.
242. The method of clause 241, wherein deploying the heating element comprises mechanically deploying the heating element.
243. The method of clause 241, wherein deploying the heating element comprises automatically deploying the heating element.
244. The method of any one of clauses 208 to 243, further comprising advancing the heating element until it contacts substantially an entire perimeter of an anterior lens capsule of the eye;
245. The method of clause 243, further comprising tilting the heating element via a gimballed joint structure to conform the contact frame to a surface of the anterior lens capsule.
246. The method of clause 243 or 245, wherein an anterior portion of the capsular bag is thermally ablated along an entire perimeter.
247. The method of any one of clauses 208 to 246, wherein the controller comprises a portable controller.
248. The method of clause 247, wherein the portable controller is battery powered and operates without connection to a large external console.
249. The method of clause 247 or 248, wherein the controller comprises a handpiece.
250. The method of any one of clauses 208 to 249, wherein the at least one direct-current pulse comprises at least one ultrashort direct-current pulse from about 10 microseconds to about 1 second in duration.
251. The method of any one of clauses 208 to 250, further comprising deploying the heating element to its shape-memory geometry.
252. The method of any one of clauses 208 to 251, further comprising extending the heating element into an anterior chamber of an eye upon deployment.
253. The method of clause 252, further comprising preventing activation of the heating element unless the heating element is confirmed to be deployed in the anterior chamber.
254. The method of any one of clauses 208 to 253, further comprising monitoring temperature of tissues adjacent to the capsular bag via a thermal sensor positioned near the heating element.
255. The method of any one of clauses 208 to 254, further comprising insulating the heating element to prevent thermal spread to an iris or corneal endothelium of the eye.
256. The method of any one of clauses 208 to 255, wherein the method is applied in human ophthalmic surgery.
257. The method of any one of clauses 208 to 255, wherein the method is applied in veterinary ophthalmic surgery.
258. The method of any one of clauses 208 to 255, wherein the method is applied in non-ophthalmic procedures comprising one or more of tympanostomy, dura opening, dermatologic excision, or endoscopic mucosal resection.
259. The method of any one of clauses 208 to 258, wherein the portion of the capsular bag comprises an anterior portion of the capsular bag.
260. The method of any one of clauses 208 to 259, wherein actuating the controller to deliver the at least one current pulse comprises actuating the controller to deliver at least one current pulse with a voltage from about 4 V to about 5 V.
261. A system for forming an opening in a capsular bag of an eye of a subject, the system comprising: a thermal device comprising a thermal device of any one of clauses 58 to 171 or an apparatus of any one of clauses 172 to 207, wherein the apparatus comprises a heating element; a control unit configured to deliver ultrashort pulses of current to the heating element; and a delivery device comprising a distal end and a handpiece, wherein the distal end of the delivery device is coupled to the heating element, wherein the delivery device is configured to change a shape of the heating element for insertion into the eye.
262. The system of clause 261, wherein the ultrashort pulses of current comprise ultrashort pulses of direct current.
263. The system of clause 261 or 262, wherein each ultrashort pulse of current is from about 10 microseconds to about 1 second in duration.
264. The system of any one of clauses 261 to 263, wherein a single pulse of current is sufficient to complete the capsulotomy.
265. The system of any one of clauses 261 to 263, wherein a plurality of sub-pulses of current are sufficient to complete the capsulotomy.
266. The system of any one of clauses 261 to 265, wherein the control unit is configured to monitor loop resistance during pulse delivery to confirm tissue ablation.
267. The system of any one of clauses 261 to 266, wherein the control unit is configured to terminate current flow once the capsulotomy is complete.
268. The system of any one of clauses 261 to 267, wherein the control unit is portable.
269. The system of any one of clauses 261 to 268, wherein the control unit is battery powered and is configured to operate without connection to a large external console.
270. The system of any one of clauses 261 to 269, wherein the control unit comprises a portable housing comprising one or more of a battery pack, pulse-generation electronics, and a user interface.
271. The system of any one of clauses 261 to 270, wherein the further comprising a closed-loop conductor at least partly coupling the heating element to the delivery device.
272. The system of clause 271, wherein the control unit is electrically coupled to the closed-loop conductor.
273. The system of clause 272, wherein the control unit is configured to deliver at least one ultrashort direct-current pulse to the closed-loop conductor while the heating element is in contact with the capsular bag.
274. The system of any one of clauses 261 to 273, wherein the heating element comprises a joint structure and a contact frame, wherein the joint structure is configured to couple the contact frame to the delivery device.
275. The system of clause 274, wherein the joint structure is configured to tilt the contact frame relative to the delivery device and to align with a surface of the capsular bag.
276. The system of clause 274 or 275, wherein one or more of the contact frame, the joint structure, or at least a portion of the distal end of the delivery device are contained in a removable tip configured for attachment to and removal from the handpiece.
277. The system of clause 276, wherein the removable tip is part of a set of removable tips provided to a medical professional for use in a capsular bag operation, wherein each removable tip comprises a contact frame defining a perimeter corresponding to an opening size from about 0.1 mm to about 10 mm.
278. The system of clause 277, wherein each removable tip defines a perimeter corresponding to the same opening size.
279. The system of clause 277, wherein at least one removable tip defines a perimeter corresponding to an opening size different from at least another removable tip of the set of removable tips.
280. The system of clause 277, wherein at least one contact frame of the set of removable tips has a diameter from about 5 mm to about 6 mm and at least one contact frame of the set of removable tips has a diameter from about 2 mm to about 3 mm.
281. The system of any one of clauses 261 to 280, wherein the delivery device comprises a tubular delivery device.
282. The system of clause 281, wherein the tubular delivery device is configured to surround at least a portion of the heating element, wherein the heating element is configured to be movable relative to the tube between an insertion configuration and a deployed configuration.
283. The system of clause 282, wherein, in the insertion configuration, the tubular delivery device is configured to fold the heating element by elongating the heating element.
284. The system of any one of clauses 261 to 280, wherein the delivery device comprises a scissor-type delivery device.
285. The system of clause 284, wherein the scissor-type delivery device comprises one or more linkages comprising the distal end of the delivery device.
286. The system of clause 285, wherein the one or more linkages are configured to move the heating element between a compact folded insertion configuration and a deployed configuration in response to actuation at a proximal portion of the handpiece.
287. The system of clause 286, wherein, in the insertion configuration, the tubular delivery device is configured to fold the heating element by squeezing the heating element.
288. A robotic system for automated capsulotomy of an eye of a subject, the robotic system comprising: a robotic arm comprising an end effector; a housing configured to be held by the end effector of the robotic arm, wherein the housing is coupled to a heating element configured to be changeable into a tubular or elliptical configuration for insertion into the eye; and a controller configured to deploy the heating element into an anterior chamber of the eye and to activate a current pulse to create a capsulotomy.
289. The robotic system of clause 288, wherein the heating element comprises a conductive loop.
290. The robotic system of clause 288 or 289, wherein the current pulse comprises an ultrashort direct-current pulse.
291. The robotic system of any one of clauses 288 to 290, wherein the capsulotomy comprises a reproducible capsulotomy without manual tearing.
292. The robotic system of any one of clauses 288 to 291, wherein the robotic arm is configured to position the heating element under computer vision guidance.
293. The robotic system of any one of clauses 288 to 292, wherein the robotic system is configured to select a heating element from a cartridge containing multiple pre-formed heating element geometries.
294. The robotic system of any one of clauses 288 to 293, wherein the robotic system is configured to perform the capsulotomy in less than about 0.1 second.
295. The robotic system of any one of clauses 288 to 294, wherein the robotic system is configured to apply a feedback-controlled pulse based on heating element resistance or temperature.
296. The robotic system of any one of clauses 288 to 295, wherein the robotic system is capable of performing anterior and posterior capsulotomies in the same surgical session.
297. The robotic system of any one of clauses 288 to 296, further comprising a robotic controller configured to position the heating element in the anterior chamber.
298. The robotic system of clause 297, wherein the robotic controller is configured to command the controller to deliver at least one ultrashort direct-current pulse to create the capsulotomy.
299. The robotic system of any one of clauses 288 to 298, further comprising multiple removable tips with heating elements having different perimeters; and a tool-change mechanism configured to allow the robotic arm to select and attach a tip of the multiple removable tips.
300. The robotic system of any one of clauses 288 to 299, wherein the heating element comprises a heating element as in any one of clauses 58 to 171 or an apparatus of any one of clauses 172 to 207.
301. The robotic system of any one of clauses 288 to 300, wherein the housing comprises a thermal device of any one of clauses 58 to 171.
302. A method of forming an opening in a capsular bag of an eye of a subject, the method comprising: changing a heating element carried by a gimballed contact frame into a tubular or elliptical configuration for insertion into the eye; conforming the gimballed contact frame to a surface of the capsular bag, wherein the gimballed contact frame self-aligns via a gimbal to maintain substantially full perimeter contact with the capsular bag within a predetermined angle range relative to an angle of a delivery device; and heating a portion of the capsular bag with the heating element to a target temperature such that tissue of the capsular bag is severed to form the opening.
303. The method of clause 302, further comprising placing the heating element proximate to or in contact with the portion of the capsular bag, wherein the portion of the capsular bag is heated via the heating element.
304. The method of clause 303, wherein the heating element is coupled to the delivery device via a closed-loop conductor, and wherein heating the portion of the capsular bag comprises applying a current through the closed-loop conductor to the heating element.
305. The method of clause 304, wherein the current comprises a direct current.
306. The method of clause 305, wherein applying the direct current to the heating element comprises applying at least one pulse of direct current.
307. The method of clause 306, further comprising detecting a temperature proximate the heating element and adjusting the application of the direct current to the heating element based on the detected temperature.
308. The method of clause 302, wherein the target temperature is from about 60°C to about 150 °C.
309. The method of clause 302, further comprising applying at least one ultrashort direct-current pulse to the heating element for a duration from about 10 microseconds to about 1 second, thereby ablating the capsular bag.
310. The method of clause 309, wherein the at least one ultrashort direct-current pulse has a voltage from about 1 V to about 20 V.
311. The method of clause 310, wherein the voltage is from about 4 V to about 5 V.
312. The method of clause 302, further comprising deploying the heating element to a deployment configuration having a predetermined perimeter.
313. The method of clause 312, further comprising deploying the heating element to its shape-memory geometry.
314. The method of clause 312, wherein the heating element is foldable into the tubular or elliptical configuration for insertion through an incision of about 3 mm or less.
315. The method of clause 302, wherein the gimbal comprises one or more of: one or more posts disposed on a delivery interface of the gimballed contact frame and extending away from a contact surface of the gimballed contact frame; one or more hinges disposed on the delivery interface and configured to provide an open area for interfacing with the delivery device; or one or more slots proximal the one or more posts and configured to receive a distal end of the delivery device.
316. The method of clause 315, further comprising actuating a portable controller to deliver at least one ultrashort direct-current pulse through a closed-loop conductor to thermally ablate an anterior lens capsule along an entire perimeter.
317. The method of clause 316, wherein the portable controller is battery powered and operates without connection to a large external console.
318. The method of clause 317, wherein the portable controller comprises a handpiece.
319. The method of clause 302, further comprising extending the heating element into an anterior chamber of the eye upon deployment.
320. The method of clause 319, further comprising preventing activation of the heating element unless the heating element is confirmed to be deployed in the anterior chamber.
321. The method of clause 302, further comprising attaching a tip comprising the heating element, a closed-loop conductor, and a gimballed joint structure to the delivery device.
322. The method of clause 321, wherein the delivery device comprises a scissor-type delivery device.
323. The method of clause 322, further comprising locking the scissor-type delivery device in at least one of a fully open configuration and a fully closed configuration via a one-click locking mechanism without continuous user-applied force on the delivery device.
324. The method of clause 322, further comprising rotating a scroll wheel to proportionally transition the heating element between a compact insertion configuration and a deployed configuration via an arced lead screw operatively coupled to the scissor-type delivery device.
325. The method of clause 302, wherein the heating element comprises one or more notches disposed at or proximate to a junction between the heating element and a weld tab, the one or more notches configured to locally increase electrical resistance of the heating element at the junction by reducing a cross-sectional area of the heating element at the junction, to reduce cold spots and improve uniformity of thermal energy delivery around a perimeter of the heating element.
326. The method of clause 302, wherein the heating element is applied to a trabecular meshwork to create a glaucoma outflow channel.
327. The method of clause 302, furthering comprising performing the method using a robotic system comprising: a robotic arm comprising an end effector; a housing configured to be held by the end effector; and a controller configured to deploy the heating element into an anterior chamber of the eye and to activate a current pulse to create a capsulotomy.
328. The method of clause 327, further comprising selecting, via a tool-change mechanism, a distal tip with the heating element from a plurality of distal tips having heating elements of one or more different shapes or sizes based on one or more of a desired shape or size for the opening.
329. The method of clause 327, further comprising positioning the heating element under computer vision guidance prior to activating the current pulse.
330. The method of clause 302, further comprising monitoring a loop resistance of the heating element during pulse delivery to confirm tissue ablation.
331. The method of clause 302, wherein the predetermined angle range is from about 5 degrees to about 90 degrees, and wherein the gimbal remains operative when the heating element is in both the tubular or elliptical configuration and a deployed configuration.
332. An apparatus for forming an opening in a capsular bag of an eye of a subject, the apparatus comprising: a frame, wherein the frame comprises: (i) a first side comprising a contact surface configured to contact the capsular bag; (ii) a second side comprising a delivery interface configured to couple to a delivery device; and (iii) a gimbal configured to tilt the frame to maintain substantially full contact with the capsular bag within a predetermined angle range relative to an angle of the delivery device when the delivery device is coupled to the delivery interface, wherein the gimbal comprises one or more of: (1) one or more posts disposed on the delivery interface and extending away from the first side; (2) one or more hinges disposed on the delivery interface and extending away from the first side, wherein the one or more hinges are configured to provide an open area for interfacing with the delivery device; or (3) one or more slots proximal the one or more posts and configured to provide a slot for a distal end of the delivery device.
333. The apparatus of clause 332, wherein the predetermined angle range is from about 5 degrees to about 90 degrees, and wherein the gimbal remains operative when the frame is in both a compact insertion configuration and a deployed configuration, such that the frame can tilt and conform to the capsular bag in either configuration.
334. The apparatus of clause 332, wherein the delivery device comprises a robotic adapter configured to be held by a robotic arm end effector, and wherein the frame is deployable under control of a robotic controller.
335. The apparatus of clause 332, further comprising the delivery device comprising an actuator configured to transition the frame between a compact insertion configuration and a deployed configuration.
336. The apparatus of clause 332, wherein the one or more hinges comprise a rounded elbow structure surrounding the open area.
337. The apparatus of clause 332, wherein the one or more slots are formed by a space between the one or more posts and the one or more hinges.
338. The apparatus of clause 332, wherein the one or more posts comprises a plurality of posts mirrored along a central axis of the frame.
339. The apparatus of clause 332, wherein the frame is configured to be inserted through an incision of about 3 mm or less in an anterior chamber of the eye.
340. The apparatus of clause 332, wherein the frame is configured to be changeable into a tubular or elliptical configuration for insertion into the eye.
341. The apparatus of clause 340, wherein the frame is configured to be changeable into the tubular or elliptical configuration via one or more of folding, compressing, or stretching a heating element.
342. The apparatus of clause 332, wherein the frame comprises a shape-memory geometry.
343. The apparatus of clause 332, wherein the contact surface comprises a heating element configured to be heated to a target temperature from about 60°C to about 150 °C to sever tissue of the capsular bag.
344. The apparatus of clause 332, wherein the frame comprises one or more notches disposed at or proximate to a junction between the frame and a weld tab or conducting wire attachment, the one or more notches configured to locally increase electrical resistance of the frame at the junction by reducing a cross-sectional area of the frame at the junction, to reduce cold spots and improve uniformity of thermal energy delivery around the contact surface.
345. The apparatus of clause 332, further comprising an anchor configured to anchor the delivery interface to the delivery device.
346. The apparatus of clause 332, further comprising one or more stabilization features configured to control the tilt of the frame.
347. The apparatus of clause 346, wherein the one or more stabilization features comprise a proximal stabilization feature configured to prevent catching of a proximal end of the frame on the eye after opening the capsular bag.
348. The apparatus of clause 332, wherein the delivery device comprises a scissor-type delivery device, the scissor-type delivery device comprising one or more linkages comprising the distal end of the delivery device.
349. The apparatus of clause 348, wherein the one or more linkages are configured to move a heating element between a compact folded insertion configuration and a deployed configuration in response to actuation at a proximal portion of a handpiece of the delivery device.
350. The apparatus of clause 348, wherein the scissor-type delivery device comprises a one-click locking mechanism comprising a locking element configured to engage a corresponding locking element opening to selectively retain the scissor-type delivery device in at least one of a fully open configuration and a fully closed configuration without continuous user-applied force.
351. The apparatus of clause 350, wherein engagement and disengagement of the locking element is accompanied by at least one of audible feedback or tactile feedback confirming that the scissor-type delivery device has reached a discrete locked or unlocked end-state.
352. The apparatus of clause 350, wherein, when the scissor-type delivery device is in the fully closed configuration, the frame is retracted posteriorly relative to the distal end of the delivery device to reduce an exposed proximal edge of the frame during withdrawal from the eye.
353. The apparatus of clause 348, further comprising a scroll wheel delivery mechanism comprising an arced lead screw operatively coupled to the scissor-type delivery device and a user-actuated scroll wheel configured to drive movement of the arced lead screw, wherein rotation of the user-actuated scroll wheel proportionally transitions the frame between a compact insertion configuration and a deployed configuration.
354. The apparatus of clause 353, further comprising at least one of an open-position detection mechanism or a closed-position detection mechanism configured to detect when the frame has reached a predefined fully open or fully closed end-position, and a feedback mechanism configured to provide at least one of haptic feedback, audible feedback, or visual feedback communicating the detected end-position to a user.
355. The apparatus of clause 332, wherein the frame and the gimbal are contained in a removable tip configured for attachment to and removal from a handpiece of the delivery device.
356. The apparatus of clause 332, further comprising a closed-loop conductor at least partly coupling a heating element to the delivery device, wherein the closed-loop conductor is configured to deliver current from a power source to the heating element for resistive heating.
357. The apparatus of clause 356, wherein the power source is configured to deliver pulses of energy with a voltage from about 1 V to about 20 V.
358. The apparatus of clause 356, further comprising a control unit comprising a portable housing comprising one or more of a battery pack, pulse-generation electronics, or a user interface, and configured to operate without connection to a large external console.
359. The apparatus of clause 358, wherein the control unit is configured to monitor a loop resistance during pulse delivery to confirm tissue ablation.
360. The apparatus of clause 359, wherein the control unit is configured to terminate current flow once a capsulotomy is complete.
361. The apparatus of clause 332, wherein activation of a heating element is prevented unless the heating element is confirmed to be deployed in an anterior chamber of the eye.

While various embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations, or equivalents. It is intended that the following claims define the scope of the disclosure and that methods and structures within the scope of these claims and their equivalents be covered thereby.

What is claimed is:

1. A method of forming an opening in a capsular bag of an eye of a subject, the method comprising:
changing a heating element carried by a gimballed contact frame into a tubular or elliptical configuration for insertion into the eye;
conforming the gimballed contact frame to a surface of the capsular bag, wherein the gimballed contact frame self-aligns via a gimbal to maintain substantially full perimeter contact with the capsular bag within a predetermined angle range relative to an angle of a delivery device; and
heating a portion of the capsular bag with the heating element to a target temperature such that tissue of the capsular bag is severed to form the opening.

2. The method of claim 1, further comprising placing the heating element proximate to or in contact with the portion of the capsular bag, wherein the portion of the capsular bag is heated via the heating element.

3. The method of claim 2, wherein the heating element is coupled to the delivery device via a closed-loop conductor, and wherein heating the portion of the capsular bag comprises applying a current through the closed-loop conductor to the heating element.

4. The method of claim 3, wherein the current comprises a direct current.

5. The method of claim 4, wherein applying the direct current to the heating element comprises applying at least one pulse of direct current.

6. The method of claim 5, further comprising detecting a temperature proximate the heating element and adjusting the application of the direct current to the heating element based on the detected temperature.

7. The method of claim 1, wherein the target temperature is from about 60 °C to about 150 °C.

8. The method of claim 1, further comprising applying at least one ultrashort direct-current pulse to the heating element for a duration from about 10 microseconds to about 1 second, thereby ablating the capsular bag.

9. The method of claim 8, wherein the at least one ultrashort direct-current pulse has a voltage from about 1 V to about 20 V.

10. The method of claim 9, wherein the voltage is from about 4 V to about 5 V.

11. The method of claim 1, further comprising deploying the heating element to a deployment configuration having a predetermined perimeter.

12. The method of claim 11, further comprising deploying the heating element to its shape-memory geometry.

13. The method of claim 11, wherein the heating element is foldable into the tubular or elliptical configuration for insertion through an incision of about 3 mm or less.

14. The method of claim 1, wherein the gimbal comprises one or more of: one or more posts disposed on a delivery interface of the gimballed contact frame and extending away from a contact surface of the gimballed contact frame; one or more hinges disposed on the delivery interface and configured to provide an open area for interfacing with the delivery device; or one or more slots proximal the one or more posts and configured to receive a distal end of the delivery device.

15. The method of claim 14, further comprising actuating a portable controller to deliver at least one ultrashort direct-current pulse through a closed-loop conductor to thermally ablate an anterior lens capsule along an entire perimeter.

16. The method of claim 15, wherein the portable controller is battery powered and operates without connection to a large external console.

17. The method of claim 16, wherein the portable controller comprises a handpiece.

18. The method of claim 1, further comprising extending the heating element into an anterior chamber of the eye upon deployment.

19. The method of claim 18, further comprising preventing activation of the heating element unless the heating element is confirmed to be deployed in the anterior chamber.

20. The method of claim 1, further comprising attaching a tip comprising the heating element, a closed-loop conductor, and a gimballed joint structure to the delivery device.

21. The method of claim 20, wherein the delivery device comprises a scissor-type delivery device.

22. The method of claim 21, further comprising locking the scissor-type delivery device in at least one of a fully open configuration and a fully closed configuration via a one-click locking mechanism without continuous user-applied force on the delivery device.

23. The method of claim 21, further comprising rotating a scroll wheel to proportionally transition the heating element between a compact insertion configuration and a deployed configuration via an arced lead screw operatively coupled to the scissor-type delivery device.

24. The method of claim 1, wherein the heating element comprises one or more notches disposed at or proximate to a junction between the heating element and a weld tab, the one or more notches configured to locally increase electrical resistance of the heating element at the junction by reducing a cross-sectional area of the heating element at the junction, to reduce cold spots and improve uniformity of thermal energy delivery around a perimeter of the heating element.

25. The method of claim 1, wherein the heating element is applied to a trabecular meshwork to create a glaucoma outflow channel.

26. The method of claim 1, furthering comprising performing the method using a robotic system comprising:
a robotic arm comprising an end effector;
a housing configured to be held by the end effector; and
a controller configured to deploy the heating element into an anterior chamber of the eye and to activate a current pulse to create a capsulotomy.

27. The method of claim 26, further comprising selecting, via a tool-change mechanism, a distal tip with the heating element from a plurality of distal tips having heating elements of one or more different shapes or sizes based on one or more of a desired shape or size for the opening.

28. The method of claim 26, further comprising positioning the heating element under computer vision guidance prior to activating the current pulse.

29. The method of claim 1, further comprising monitoring a loop resistance of the heating element during pulse delivery to confirm tissue ablation.

30. The method of claim 1, wherein the predetermined angle range is from about 5 degrees to about 90 degrees, and wherein the gimbal remains operative when the heating element is in both the tubular or elliptical configuration and a deployed configuration.
